SEQUENCE LISTING (7 sequences)

SEQ ID NO: 1 - protein, 16 aa, Artificial Sequence
  Rac1 Inhibitor W56
MVDGKPVNLGLWDTAG

SEQ ID NO: 2 - RNA, 22 nt, Artificial Sequence
  miR29c
uagcaccauuugaaaucgguua

SEQ ID NO: 3 - RNA, 22 nt, Artificial Sequence
  miR29c
ugaccgauuucuccugguguuc

SEQ ID NO: 4 - RNA, 20 nt, Artificial Sequence
  miR29c
uagcaccauuugaaaucggu

SEQ ID NO: 5 - RNA, 88 nt, Artificial Sequence
  mmu-miR-29c-5p (miR29c inhibitor)
aucucuuacacaggcugaccgauuucuccugguguucagagucuguuuuugucuagcaccauuugaaaucgguuaugauguaggggga

SEQ ID NO: 6 - DNA, 18 bp, Artificial Sequence
  b-actin forward primer
cactgtcgagtcgcgtcc

SEQ ID NO: 7 - DNA, 20 bp, Artificial Sequence
  b-actin reverse primer
tcatccatggcgaactggtg

(19) United States
(12) Patent Application Publication
(10) Pub. No.: US 2023/0263800 A1
(43) Pub. Date: Aug. 24, 2023

(54) COMPOSITIONS AND METHODS TO PROMOTE THYMIC FUNCTION

(71) Applicant: Fred Hutchinson Cancer Center, Seattle, WA (US)

(72) Inventors: Jarrod Dudakov, Seattle, WA (US); Sinead Kinsella, Seattle, WA (US); Lorenzo Iovino, Seattle, WA (US)

(73) Assignee: Fred Hutchinson Cancer Center, Seattle, WA (US)

(21) Appl. No.: 18/043,303

(22) PCT Filed: Nov. 3, 2021
(86) PCT No.: PCT/US21/57924

§ 371 Date: Feb. 27, 2023

Related U.S. Application Data
(60) Provisional application No. 63/109,271, filed on Nov. 3, 2020.

(51) Int. Cl. A61K 31/506; A61K 31/675; A61K 31/365; A61K 31/351; A61K 31/4184; A61P 3/00

(52) U.S. Cl. CPC A61K 31/506; A61K 31/675; A61K 31/365; A61K 31/351; A61K 31/4184; A61P 3/00

(57) ABSTRACT

Compositions and methods to promote thymic function are described. The compositions and methods can activate the GPR39 receptor and/or a purinergic receptor, such as P2Y2. The activation can upregulate regenerative molecules, such as FOXN1, interleukin (IL)-22, IL-23, and bone morphogenetic protein 4 (BMP4).

### CROSS-REFERENCE TO RELATED APPLICATIONS

This application is a U.S. National Phase Application which claims priority to International Patent Application No. PCT/US2021/057924 filed Nov. 3, 2021, which claims priority to U.S. Provisional Patent Application No. 63/109,271, filed Nov. 3, 2020, the contents of both of which are incorporated herein by reference in their entirety as if fully set forth herein.

### STATEMENT REGARDING FEDERALLY SPONSORED RESEARCH OR DEVELOPMENT

This invention was made with government support under grant HL145276 awarded by the National Institutes of Health. The government has certain rights in the invention.

### REFERENCE TO SEQUENCE LISTING

The Sequence Listing associated with this application is provided in text format in lieu of a paper copy and is hereby incorporated by reference into the specification. The name of the text file containing the Sequence Listing is 2UH2633_ST25.txt. The text file is 1.72 KB, was created on Feb. 27, 2023, and is being submitted electronically via Patent Center.

### FIELD OF THE DISCLOSURE

The disclosure provides compositions and methods to promote thymic function. The compositions and methods can activate G-protein coupled receptor 39 (GPR39) and/or a purinergic receptor, such as P2Y2. The activation can upregulate regenerative molecules, such as FOXN1, interleukin (IL)-22, IL-23, and bone morphogenetic protein 4 (BMP4).

### BACKGROUND OF THE DISCLOSURE

The thymus is a specialized organ responsible for the generation and maintenance of T cells, a major component of the adaptive immune system. T cell development is a complicated process that requires the close interaction between hematopoietic precursors and the thymic stromal microenvironment, which includes thymic epithelial cells (TECs), fibroblasts, and endothelial cells. These interactions drive the commitment, proliferation, and differentiation of hematopoietic precursors imported from the circulation in a tightly regulated process. TECs in particular are critical regulators of all critical stages of T cell development including the selection and tolerance of the T cell receptor repertoire.

However, despite its importance for the generation of a diverse naïve T cell repertoire, the thymus is extremely sensitive to injury such as that caused by infection, shock, or common cancer therapies such as cytoreductive chemo- or radiation therapy. It also, however, has a remarkable capacity for endogenous repair. Nevertheless, even though there is continual thymic involution and regeneration in response to everyday insults like stress and infection, profound thymic damage caused by common cancer therapies and the conditioning regimens used as part of hematopoietic cell transplantation (HCT), leads to prolonged T cell deficiency, precipitating high morbidity and mortality from opportunistic infections and likely facilitating malignant relapse. Furthermore, this capacity for regeneration declines as a function of age-related thymic involution.

### SUMMARY OF THE DISCLOSURE

The disclosure provides compositions and methods to promote thymic regeneration. The compositions and methods can activate G-protein coupled receptor 39 (GPR39) and/or a purinergic receptor, such as P2Y2. Other purinergic receptors for activation include P2Y1, P2Y14, P2Y6, P2X7, P2X3, P2X4, P2X1, and P2X6. The activation can upregulate regenerative molecules, such as FOXN1, interleukin (IL)-22, IL-23, and bone morphogenetic protein 4 (BMP4).

### BRIEF DESCRIPTION OF THE SEVERAL VIEWS OF THE DRAWINGS

Some of the drawings submitted herein may be better understood in color. Applicant considers the color versions of the drawings as part of the original submission and reserves the right to present color images of the drawings in later proceedings.

FIGS. 1A-1F. Dietary deficiency of zinc rapidly impairs T cell development. 6-8-week-old female C57BL16 mice were fed a normal or Zn-deficient (ZD) diet for up to 8 weeks. (1A) Total thymus cellularity from untreated mice or after 21 or 56 days of ZD (untreated, n=24, combined from animals harvested alongside either day 21 or day 56 mice; day 21, n=15 over three independent experiments; day 56, n=10 over two independent experiments). (1B) Concatenated flow cytometry plots displaying CD4 and CD8 expression in the thymus (plots were gated on viable CD45+ cells). (1C) Total number of CD4-CD8-Thy1+ double negative (DN), CD3-CD4-CD8⁺ intermediate single positive (iSP), CD4+CD8⁺ double positive (DP), CD3+CD8-CD4+(SP4), or CD3+CD8+CD4⁻ (SP8) thymocytes from untreated mice or after 21 or 56 days of ZD (untreated, n=24, combined from animals harvested alongside either day 21 or day 56 mice; day 21, n=15 over three independent experiments; day 56, n=10 over two independent experiments). (1D) Multi parameter flow cytometry data from untreated or 21 days after ZD was placed in Uniform Manifold Approximation & Projection (UMAP) space and clusters were generated based on relative mean fluorescence intensity (MFI) of markers of thymocyte maturation (CD25, CD44, Thy1, CD4, CD8, CD3). (1E) Concatenated flow cytometry plots showing Ki67 expression in DN3, DN4, iSP, and DP thymocytes from untreated mice or after 21 or 56 days of ZD. (1F) Lineage-depleted bone marrow cells were isolated from untreated 6-week-old C57BL/6 mice and co-cultured with OP9-DL1 cells for 30 days in the presence or absence of zinc sulphate (ZnSO₄) (10 μM added from day 0). Concatenated flow cytometry plots displaying CD4 and CD8 expression at day 30 (n=5 independent experiments). Graphs represent mean±standard error of mean (SEM), each dot represents a biologically independent observation. *, p<0.05; **, p<0.01; ***p<0.001.

FIG. 2A-2E. 6-8-week-old female C57BL/6 mice were fed a normal or ZD diet for up to 56 days. (2A) Photos of the thymus from mice fed either control diet or ZD diet for 21 days. (2B) Weight of thymuses isolated after 21 days of ZD diet. (2C) Absolute lymphocyte counts (ALC) on the peripheral blood after 21 days of ZD diet. (2D) Proportion of naïve CD4⁺ or CD8⁺ T cells (as a proportion of total CD4⁺ or CD8⁺ T cells) after 1, 3, 5, or 8 weeks of ZD diet. (2E) Concentration of cortisol in serum of mice that had received control diet (ctrl), and after 1, 3, 5, and 8 weeks of ZD diet. Graphs represent mean±SEM, each dot represents a biologically independent observation. *, p<0.05; **, p<0.01; ***p<0.001

FIG. 3. 6-8-week-old female C57BL/6 mice were fed a normal or ZD diet for up to 8 weeks. Total number of CD4⁻CD8⁻CD3⁻ DN thymocytes from untreated mice or after 21 or 56 days of ZD: CD44⁺CD25⁻ DN1, CD44⁺CD25⁻c-kit⁺ early thymic progenitors (ETP), CD44⁺CD25⁺ DN3, or CD44⁻CD25⁻CD90⁺ DN4 (untreated, n=24, combined from animals harvested alongside either day 21 or day 56 mice; day 21, n=15 over three independent experiments; day 56, n=10 over two independent experiments). Graphs represent mean±SEM, each dot represents a biologically independent observation. *, p<0.05; **, p<0.01; ***p<0.001.

FIGS. 4A-4G. Dietary zinc intake dictates regenerative capacity of the thymus after damage. (4A, 4B) 6-8 week-old female C57BL/6 mice were fed a normal or ZD diet for 21 days at which point mice were given a sublethal dose of total body irradiation (TBI, 550 cGy). (4A) Total thymic cellularity at days 7 and 14 after TBI (n=10/treatment/timepoint across two independent experiments). (4B) Total number of CD45-EpCAM⁺MHCII⁺UEA1^{lo}Ly51^{hi} (cTECs) and CD45⁻ EpCAM⁺MHCII⁺UEA1^{hi} Ly51^{lo} (mTECs) at days 7 and 14 after TBI (n=10/treatment/timepoint across two independent experiments). (4C) 6-12 week-old female BALB.B mice were fed with normal or ZD diet for 21 days, at which point mice were given a lethal dose of TBI (900cGy) and 10×10⁶ T cell-depleted bone marrow (BM) cells from 6-8 week-old C57BL/6. One cohort also received 2×10⁶ T cells to induce graft versus host disease (GVHD); thymic cellularity was quantified on day 14 after allogeneic hematopoietic stem cell transplant (allo-HCT, n=5-6/group). (4D) 6-8 week-old female C57BL/6 mice were fed with normal or ZD diet for 21 days at which point mice were given 550 cGy TBI and total thymus cellularity quantified on day 14. One cohort was given supplemental Zn in drinking water (300 mg/kg/day ZnSO₄) from day 0 until analysis on day 14 (n=10/group across two independent experiments). (4E, 4F) 6-8 week-old female C57BL/6 female mice were given supplemental Zn in drinking water (300 mg/kg/day ZnSO₄) for 21 days at which point mice were given 550 cGy TBI. Mice were maintained on ZnSO₄ in drinking water and the thymus was analyzed on day 0 or 7 (Day 0: untreated, n=22; ZD, n=10 across two independent experiments; Day 7: untreated, n=15; ZD, n=15, across three independent experiments). (4E) Total thymic cellularity. (4F) Total number of cTECs and mTECs. (4G) Total number of Ki-67+ thymic endothelial cells (TECs). Graphs represent mean±SEM, each dot represents a biologically independent observation. *, p<0.05; **, p<0.01; ***p<0.001.

FIG. 5. 6-8 week-old female C57BL/6 mice were fed a normal or ZD diet for 21 days at which point mice were given a sublethal dose of TBI (550cGy). Absolute number of DN, DP, SP4, and SP8 thymocyte subsets was calculated on day 7 and day 28 after TBI. Graphs represent mean±SEM, each dot represents a biologically independent observation. *, p<0.05; **, p<0.01; ***p<0.001.

FIG. 6. 6-8-week-old C57BL/6 female mice were given supplemental Zn in drinking water (300 mg/kg/day ZnSO₄) for 21 days at which point mice were given 550 cGy TBI. Mice were maintained on Zn-supplemented drinking water for the duration of the study. Absolute number of DN, DP, SP4, and SP8 thymocyte subsets was calculated on day 7 after TBI. Graphs represent mean±SEM, each dot represents a biologically independent observation. *, p<0.05; **, p<0.01; ***p<0.001.

FIGS. 7A, 7B. (7A) Thymic epithelial cell lines (C9, cTEC; TE-71, mTEC) were cultured with 100 μM ZnSO₄ for 24 h when Foxn1 expression was quantified by quantitative polymerase chain reaction (qPCR, n=3 independent experiments). (7B) C9 or TE-71 cells were incubated with graded doses of ZnSO₄ for 24 h after which proliferation was assessed. Graphs represent mean±SEM, each dot represents a biologically independent observation (n=3 independent experiments). *, p<0.05; **, p<0.01; ***p<0.001.

FIGS. 8A-8F. Zinc stimulates the production bone morphogenetic protein 4 (BMP4) by endothelial cells. (8A) 6-8 week-old female C57BL/6 mice were fed a normal or ZD diet for 21 days at which point mice were given 550 cGy TBI. One cohort was given supplemental Zn in drinking water (300 mg/kg/day ZnSO₄) from day 0. Levels of BMP4 were quantified by enzyme-linked immunoassay (ELISA) at day 10 after TBI (n=5-10/group from one independent experiment). (8B, 8C) 6-8-week-old C57BL/6 female mice were given supplemental Zn in drinking water (300 mg/kg/day ZnSO₄) for 21 days at which point mice were given 550 cGy TBI. Mice were maintained on Zn-supplemented drinking water for the duration of the study. (8B) BMP4 levels measured by ELISA at day 10 (n=9/group combined from three independent experiments). (8C) Endothelial cells (ECs) were fluorescence-activated cell sorting (FACS) purified at day 7 and Bmp4 expression was measured by qPCR (n=6/group combined from two independent experiments). (8D, 8E) Exogenous endothelial cells (exECs) were generated as previously described (Wertheimer et al., Science Immunology. 2018, 3(19); and Seandel et al., Proc Natl Acad Sci USA. 2008, 105(49):19288-19293) and stimulated for 24 hours with ZnSO₄ at the indicated concentrations and Bmp4 expression measured by qPCR at 24 hours (50 μM: n=8/group combined from three independent experiments; 100 μM: n=11/group across five independent experiments) (8D) and BMP4 protein was quantified by ELISA at 48 hours (n=3 independent experiments) (8E). (8F) 6-8 week-old female C57BL/6 mice were given supplemental Zn in drinking water (300 mg/kg/day ZnSO₄) for 21 days at which point mice were given 550cGy TBI. Mice were administered with the BMP type I receptor inhibitor Dorsomorphin dihydrochloride (12.5 mg/kg) by intraperitoneal (ip) injection at day −1 before TBI and twice daily after TBI and all mice were maintained on ZnSO₄ in drinking water for the duration of the study. Total thymus cellularity was quantified at day 10 after TBI (n=5/group from one independent experiment). Graphs represent mean±SEM, each dot represents a biologically independent observation. *, p<0.05; **, p<0.01; ***p<0.001.

FIGS. 9A-9G. Zn accumulates in thymocytes and is released after damage. (9A, 9B) 6-8 week-old female C57BL/6 mice were given 550 cGy TBI and levels of Zn were measured by inductively coupled plasma mass spectrometry (ICP-MS). (9A) Total thymic amounts of Zn from both intracellular and extracellular fractions of thymus (n=6/timepoint). (9B) Extracellular Zn was measured only in thymic supernatants and the ratio of extracellular to total thymic Zn was calculated (n=6/timepoint). (9C) 6-8 week-old female C57BL/6 mice were given supplemental Zn in drinking water (300 mg/kg/day ZnSO₄) for 21 days at which point one cohort was given 550 cGy TBI. Thymocytes were isolated either before or 48 hours after TBI and co-cultured with exECs. Bmp4 expression was measured by qPCR at 24 hours (n=3-4/group). (9D) 6-8 week-old C57BL/6 female mice were given supplemental Zn in drinking water (300 mg/kg/day ZnSO₄) for either 21 days before TBI, or from the day of TBI and maintained on ZnSO₄ in drinking water for the duration of the study. Thymus cellularity was measured at day 28 after TBI (n=4-5/group). (9E) 6-8 week-old female C57BL16 mice were fed a normal or ZD diet for 21 days after which thymocytes were isolated by CD90+ magnetic separation. Intracellular Zn levels were measured by staining with Fluozin-3 and assessed by flow cytometry (n=5/group across two independent experiments). (9F, 9G) 6-8 week-old female C57BL16 mice were given supplemental Zn in drinking water (300 mg/kg/day ZnSO₄) for 21 days after which thymocytes were isolated by CD90+ magnetic separation and Zn measured by staining with Fluozin-3 (9F) or ICP-MS (9G). Graphs represent mean±SEM, each dot represents a biologically independent observation. *, p<0.05; **, p<0.01; ***p<0.001.

FIGS. 10A-10F. G-protein coupled receptor 39 (GPR39) expressed by thymic endothelial cells is the central mediator of Zn-centered regeneration. (10A) exECs were stimulated for 24 hours with ZnSO₄ (100 μM) with or without the Zn ionophor pyrythione. Bmp4 expression was measured by qPCR (n=6 combined from two independent experiments). (10B) GPR39 expression across subsets in the thymus by flow cytometry at baseline. Displayed are concatenated plots from one experiment. (10C) Expression of GPR39 on cTECs, mTECs, ECs and fibroblasts at days 0, 4, and 7 after TBI. Displayed are concatenated plots from one experiment. (10D) exECs were stimulated for 24 hours with ZnSO₄ (100 μM) with or without the ERK inhibitor FR180204. BM P4 was measured by ELISA. (10E) GPR39 expression was silenced in exECs by siRNA and stimulated for 24 hours with ZnSO₄ (100 μM) after which Bmp4 expression was measured by qPCR (n=3/group). (10F) Bmp4 expression in exEC cultured for 24 hours in presence of ZnSO₄ (100 μM) and/or the GPR39 agonist TC-G 1008 (25 μM) (n=5-15 combined from five independent experiments). Graphs represent mean±SEM, each dot represents a biologically independent observation. *, p<0.05; **, p<0.01; ***p<0.001.

FIGS. 11A-110. (11A) Western blot showing expression of GPR39 on whole thymus tissue and in thymic exECs. Skeletal muscle was used as negative control and intestine as positive control. (11B) Thymic non-hematopoietic stromal cells were isolated from 6-week-old female C57BL/6 mice using CD45 Magnetic Activated Cell Sorting (MACS) cell depletion at days 0, 4, and 7 after a single dose sub-lethal total body radiation (SL-TBI) and microarray analysis performed as previously described (Wertheimer et al., 2018) (GSE106982). Displayed is the differential gene expression fold-change of Gpr39 comparing day 4 to day 0 or day 7 to day 0 (n=3; CD45⁻ cells were pooled from 3-4 mice/n). (11C) Gpr39 expression measured by qPCR in exECs after silencing with siRNA. Graphs represent mean±SEM.

FIGS. 12A-12D. Experimental targeting of the GPR39 receptor improves thymic repair and T cell reconstitution after allo-HCT. (12A, 12B) 6-8-week-old male 057BL/6 mice were given supplemental Zn in drinking water (300 mg/kg/day ZnSO₄) for 21 days at which point mice were given a lethal dose of TBI (2×550 cGy) along with T cell depleted BM from female 057BL/6 (12A) or RAG2-GFP (recombination activating gene2—green fluorescent protein) (12B) mice. Mice were maintained on ZnSO₄ in drinking water for the duration of the study. (12A). Total thymic cellularity is shown at days 7, 21, 28, and 42 after HCT (n=5-10/group/timepoint combined from two independent experiments). (12B) Splenic T cells were analyzed for GFP expression on day 53 (n=4-5/group). (12C) 6-8-week-old C57BL/6 female mice were given 550 cGy TBI and either vehicle or TC-G 1008 (20 mg/mouse/day) by guided feeding daily from day 0 until day 10 when thymus cellularity was assessed (n=8-9 combined from two independent experiments). (12D) 6-8-week-old female BALB.B mice were given a lethal dose of TBI (900cGy) along with 10×10⁶ T cell depleted BM from female C57BL/6 mice and either vehicle or TC-G 1008 (20 mg/mouse/day) by guided feeding daily from day 0 until day 8, then on day 10 and 12. Thymuses were harvested and analyzed at day 14 (n=5/group). Graphs represent mean±SEM, each dot represents a biologically independent observation. *, p<0.05; **, p<0.01; ***p<0.001.

FIGS. 13A, 13B. 6-8-week-old male C57BL/6 mice were given supplemental Zn in drinking water (300 mg/kg/day ZnSO₄) for 21 days at which point mice were given a lethal dose of TBI (2×550cGy) along with T cell depleted BM from female C57BL/6 mice. Mice were maintained on ZnSO₄ in drinking water for the duration of the study (n=5-10/group/− timepoint combined from two independent experiments). (13A) Total number of cTECs and mTECs at days 7, 21, 28, and 42 after allo-HCT. (13B) Total number of DN, DP, SP4, and SP8 thymocytes at days 7, 21, 28, and 42 after allo-HCT. Graphs represent mean±SEM, each dot represents a biologically independent observation.

FIG. 14. Male C57BL/6 mice aged 2 mo, 12 mo or 19 mo were given the GPR39 agonist TC-G 1008 (20 mg/kg/day) for 5 days. Thymus cellularity was measured at day 7.

FIGS. 15A-15E. (15A) Dendritic cells (DCs) were isolated from thymus and incubated with ZnSO₄ (100 uM) for 24 hours when the interleukin (IL)-23 subunits Il23p19 and Il12p40 were assessed by qPCR. (15B, 15C) GPR39 expression by flow cytometry in DC at days 0, 4 and 7 after TBI. (15B) Proportion of DCs expressing GPR39 at the indicated timepoints after SL-TBI. (15C) GPR39 MFI on DCs at the indicated timepoints after SL-TBI. (15D) Freshly isolated thymic DCs were incubated with TC-G 1008 (25 μM) for 24 hours when 1112p40 was assessed by qPCR. (15E) 6 wo C57BL/6 female mice were given SL-TBI (550 cGy) and vehicle or TC-G 1008 (20 mg/mouse/day) by guided feeding daily from day 2 until day 7. IL-23 levels were assessed on day 10. Graphs represent mean±SEM. *p<0.05; **p<0.01; ***p<0.001.

FIGS. 16A-16C. (16A) Thymocytes were harvested from untreated mice and incubated with either dexamethasone (100 nM) or z-VAD-FMK (zVAD, 20 μM) for 4 h prior to washing and incubation with thymic ECs or DCs. Bmp4 expression was measured by qPCR, IL12p40+ DCs were measured by flow (n=5-8). (16B) Thymic phosphatidylserine (PS) levels were assessed by flow after SL-TBI (n=4). (16C) ECs or thymic DCs were co-cultured with apoptotic thymocytes in the presence or absence of TAM inhibitor RXDX-106 (50 mM). Bmp4 expression was measured by qPCR and IL12p40+ DCs were measured by flow (n=4-5). Graphs represent mean+/−SEM.

FIGS. 17A-17L. (17A-17D) Female 1-2 mo C57BL16 mice were given 550 cGy TBI and assessed on the indicated timepoints. (17A) Proportion of baseline thymus cellularity (n=10/group). (17B) Cleaved-caspase 3 and activated Caspase-1 in CD4+CD8+ double positive thymocytes (n=7/group). (17C) Levels of lactate dehydrogenase (LDH) and high mobility group box protein 1 (HMGB1) in the extracellular millieu after TBI. (17D) Cleaved Gasdermin D measured in DP thymocytes (n=4-5). (17E) Thymuses were harvested at d0, d2, d4, and d7 after SL-TBI and tumor necrosis factor alpha (TNFα) and HMGB1 levels were measured in thymic supernatant by ELISA (n=4-5 mice/group); (17F) DCs or C9s were co-cultured with control or Nigericin treated thymocytes and IL12p40 was measured in DCs by flow; Foxn1 was quantified in C9s by qPCR. Graphs represent mean+/−SD. (17G) Thymocytes were isolated by mechanical dissociation of thymic tissue at day 0 or 2 after TBI and co-cultured with exECs. Bmp4 expression was measured by qPCR 24 hours after. (17H) Thymocytes were isolated from untreated female C57BL/6 mice and incubated for 4 hours with Nigericin (to induce pyroptosis). After 4 hours, pyroptotic thymocytes (Pyr. Thy.) were co-cultured with C9 cells (a cTEC cell line) for 24 hours after which Foxn1 expression was measured by qPCR. (17I) C9 cells were incubated with bzATP (also known as 3′-O-(4-benzoyl)benzoic adenosine triphosphate) for 24 hours after which expression of Foxn1 was assessed by qPCR. (17J, 17K) cTECs, MHC-11′° and MHC-II^{hi} mTECs were FACS purified from untreated 6 week old 057BL/6 mice at days 0, 4, and 7 after a sublethal dose of TBI (550 cGy) and transcriptomes assessed by bulk RNA sequencing. (17J) Displayed are reads for members of the P2 receptor family at day 0. (17K) Expression of P2X and P2Y receptors at day 0, 4, and 7 after TBI in cTECs. (17L) C9 cells were incubated with bzATP for 24 hours or with the inhibitor for P2Y2, after which expression of Foxn1 was assessed by qPCR. Graphs represent mean±SEM. *p<0.05; **p<0.01; ***p<0.001.

FIGS. 18A-18G. Thymuses were harvested at d0, d1, d2, and d3 after TBI (550 cGy) and (18A) mitochondrial membrane potential, (18B) Reactive oxygen species (ROS) levels, and (18C) glutathione levels were measured in DP thymocytes and cTECs (n=4-7 mice from 1-2 experiments). (18D) Foxn1 expression in FACs-purified cTECs at d0, d4, and d7 after TBI; (18E) Intracellular Ca2+ levels were measured on whole thymus harvested at d0, d2, d4, and d7 post SL-TBI. (18F, 18G) Foxn1 expression levels were measured by qPCR in (18F) C9, and (18G) TE71 cells after treatment with Tunicamycin (5 ug/ml) or Thapsigargan (1 μg/ml). Graphs represent mean+/−SD.

FIGS. 19A, 19B. (19A) C9s were treated with (19A) ATP and P2X7 antagonist or (19B) P2Y2 agonist or antagonist and FOXN1 expression was assessed by qPCR after 20 h. Graphs represent mean+/−SEM.

FIGS. 20A-20D. (20A) Levels of Zn2+ measured by mass spectrometry on thymuses harvested at d0, d1, d4, d7, and d10 after SL-TBI (ratio of total thymic Zn to extracellular fraction); (20B) ECs were incubated ex vivo with ZnSO₄ and Bmp4 gene and protein levels were measured by qPCR or ELISA (supernatants); (20C) ECs were stained with Ca2+ dye Fluo3-AM and imaged by live microscopy 20 mins post stimulation with ZnSO₄ or the GPR39 agonist TC-G 1008 (Ca2+ chelator BAPTA as negative control), area under the curve (AUC) calculated; (20D) GPR39 cell surface levels in thymocytes and TECs at d0. Graphs represent mean+/−SEM.

FIGS. 21A-21C. (21A) 2 mo female C57BL16 mice were given sublethal TBI (550 cGy) and at day 3 either received 5 mg/kg bzATP or vehicle intraperitoneally (IP). Thymus cellularity was measured at day 14. (21B) 2 month old female C57BL16 mice were given sublethal TBI (550 cGy) and at day 3 either received 5 mg/kg of the P2Y2 agonist MRS 2768 (5 mg/kg) or vehicle IP. Thymus cellularity was measured on day 14. (21C) cTEC proportions in mice treated with either P2Y2 agonist (MRS 2768, 5 mg/kg) or antagonist (AR-C 118925XX, 10 mg/kg) measured at d13 following TBI. Graphs represent mean+/−SEM.

FIGS. 22A, 22B. 6 week old mice were fed either a ZD or ZS diet for 21 days prior to TBI and (22A) total thymus cellularity was measured at d7 and d28 after TBI or (22B) proportions were quantified at d7 following TBI (n=10 mice/group).

FIG. 23. Schematic showing the proposed mechanism of endogenous thymic regeneration. (A) During steady-state T cell development, apoptotic thymocytes suppress the production of the regenerative factors IL-23 and BMP4 via detection of exposed phosphatidylserine by TAM receptors and downstream activation of Rac1, NOD2, and miR29c. TBI-induced depletion of thymocytes (and PtdSer) attentuates this suppression (Kinsella et al., 2021 Cell Reports 37: 109789). (B) After damage, there is also a switch toward immunogenic cell death (ICD), and the resulting release of damage-associated molecular patterns (DAMPs) like ATP and Zn. (C) when released during ICD, ATP can directly target TECs through P2 receptors to activate FOXN1, a crucial TEC transcription factor. (D) When Zn is released during ICD, it signals through GPR39 on ECs and DCs to promote their production of the regenerative factors BMP4 and IL-23, respectively. (E) The target of these pathways are TECs; fundamental stromal cells supporting T cell development.

### DETAILED DESCRIPTION

The thymus is a specialized organ responsible for the generation and maintenance of T cells, a major component of the adaptive immune system. T cell development is a complicated process that requires the close interaction between hematopoietic precursors and the thymic stromal microenvironment, which includes thymic epithelial cells (TECs), fibroblasts, and endothelial cells. These interactions drive the commitment, proliferation, and differentiation of hematopoietic precursors imported from the circulation in a tightly regulated process. TECs in particular are critical regulators of all critical stages of T cell development including the selection and tolerance of the T cell receptor repertoire.

However, despite its importance for the generation of a diverse naïve T cell repertoire, the thymus is extremely sensitive to injury such as that caused by infection, shock, or common cancer therapies such as cytoreductive chemo- or radiation therapy. It also, however, has a remarkable capacity for endogenous repair. Nevertheless, even though there is continual thymic involution and regeneration in response to everyday insults like stress and infection, profound thymic damage caused by common cancer therapies and the conditioning regimens used as part of hematopoietic cell transplantation (HCT), leads to prolonged T cell deficiency, precipitating high morbidity and mortality from opportunistic infections and likely facilitating malignant relapse. Furthermore, this capacity for regeneration declines as a function of age-related thymic involution.

The current disclosure describes activation of G-protein coupled receptor 39 (GPR39) and/or activation of a purinergic (e.g., P2Y2) receptor to promote thymic regeneration.

GPR39 is in the ghrelin receptor family and encodes a rhodopsin-type G-protein-coupled receptor (GPCR). Specifically, GPR39 is a class of guanine nucleotide-binding proteins that detect changes in extracellular Zn²⁺ and is involved in zinc-dependent signaling in epithelial tissue in intestines, prostate, and salivary glands. Regarding activation of GPR39 to promote thymic function, the current disclosure shows that dietary zinc supplementation can improve thymic regeneration after acute injury and in aged mice. Without being bound by theory, the mechanism by which this acts is via accumulation in thymocytes (where it is needed for normal T cell development). After acute injury, however, stored zinc is released into the extracellular milieu where it is able to stimulate the cell surface zinc receptor GPR39, ultimately stimulating the thymic regenerative factors interleukin (IL)-22, IL-23, and bone morphogenetic protein (BM P4) in lymphoid cells, dendritic cells, and endothelial cells, respectively.

Also without being bound by theory, a proposed mechanism by which dietary Zn supplementation promotes thymic regeneration involves a significant period before transplantation in order for thymocytes to accumulate Zn to be released during immunogenic cell death (ICD) after injury, thereby allowing signaling through GPR39/Ca2+/ERK in regeneration-initiating endothelial cells (ECs) and dendritic cells (DCs). The ease of Zn administration makes modulation of this pathway an attractive therapeutic target. However, more direct methods to achieve the same result are also described. As one example, data described herein shows that (1) stimulation of GPR39 with a selective agonist (TC-G 1008) induces expression of BMP4; (2) stimulation of GPR39 results in considerably greater expression of BMP4 than zinc sulphate (ZnSO₄); and (3) stimulation of GPR39 in vivo enhances thymic function in models of both acute and chronic injury (exemplified by the total body irradiation and/or allogeneic hematopoietic stem cell transplant as a model of acute injury, and age as a model of chronic injury). This makes activation (e.g., direct activation) of GPR39 a therapeutic target to mediate thymic regeneration.

An example of an agonist of the GPR39 receptor includes TC-G 1008. TC-G 1008 has a molecular formula of C₁₈H₁₉ClN₆O₂S, and IUPAC name of N-[3-chloro-4-[[[2-(methylamino)-6-pyridin-2-ylpyrimidin-4-yl]amino]methyl]phenyl]methanesulfonamide. TC-G 1008 has the following structure:

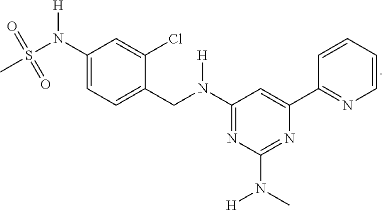 [SMILES: CNc1nc(NCc2ccc(NS(C)(=O)=O)cc2Cl)cc(-c2ccccn2)n1]

Additional examples of agonists of the GPR39 receptor include LY2784544, GSK2636771, obestatin, AZ1395, AZ4237, AZ4502, AZ9309, AZ2097 and AZ7914. LY2784544 (also known as Gandotinib) has a molecular formula of C₂₃H₂₅ClFN₇O and IUPAC name of 3-[(4-chloro-2-fluorophenyl)methyl]-2-methyl-N-(5-methyl-1H-pyrazol-3-yl)-8-(morpholin-4-ylmethyl)imidazo[1,2-b]pyridazin-6-amine. LY2784544 has the following structure:

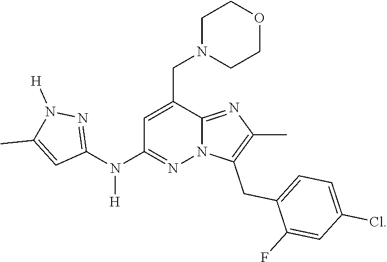 [SMILES: Cc1cc(Nc2cc(CN3CCOCC3)c3nc(C)c(Cc4ccc(Cl)cc4F)n3n2)n[nH]1]

The agonist of the GPR39 receptor, GSK2636771, has a molecular formula of C₂₂H₂₂F₃N₃O₃ and IUPAC name of 2-methyl-1-[[2-methyl-3-(trifluoromethyl)phenyl]methyl]-6-morpholin-4-ylbenzimidazole-4-carboxylic acid. GSK2636771 has the following structure:

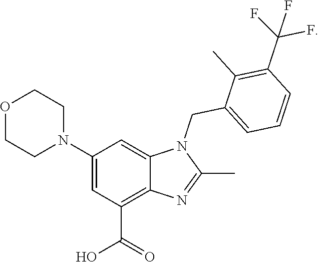 [SMILES: Cc1c(Cn2c(C)nc3c(C(=O)O)cc(N4CCOCC4)cc32)cccc1C(F)(F)F]

The agonist of the GPR39 receptor, obestatin, has a molecular formula of C₁₁₄H₁₇₄N₃₄O₃₁ and IUPAC name of (3S)-4-[[(2S)-1-[[2-[[(2S,3S)-1-[[(2S)-6-amino-1-[[(2S)-1-[[(2S)-1-[[2-[[(2S)-1-[[(2S)-5-amino-1-[[(2S)-1-[[(2S)-5-amino-1-[[(2S)-5-amino-1-[[(2S)-1-[[2-[[(2S)-1-[[(2S)-1- [[(2S)-1-amino-4-methyl-1-oxopentan-2-yl]amino]-1-oxopropan-2-yl]amino]-5-carbamimidamido-1-oxopentan-2-yl]amino]-2-oxoethyl]amino]-3-(1H-imidazol-4-yl)-1-oxopropan-2-yl]amino]-1,5-dioxopentan-2-yl]amino]-1,5-dioxopentan-2-yl]amino]-3-(4-hydroxyphenyl)-1-oxopropan-2-yl]amino]-1,5-dioxopentan-2-yl]amino]-1-oxopropan-2-yl]amino]-2-oxoethyl]amino]-3-hydroxy-1-oxopropan-2-yl]amino]-4-methyl-1-oxopentan-2-yl]amino]-1-oxohexan-2-yl]amino]-3-methyl-1-oxopentan-2-yl]amino]-2-oxoethyl]amino]-3-methyl-1-oxobutan-2-yl]amino]-3-[[2S)-2-[[(2S)-1-[(2S)-2-[[(2S)-4-amino-2-[[(2S)-2-amino-3-phenylpropanoyl]amino]-4-oxobutanoyl]amino]propanoyl]pyrrolidine-2-carbonyl]amino]-3-phenylpropanoyl]amino]-4-oxobutanoic acid. Obestatin has the following structure:

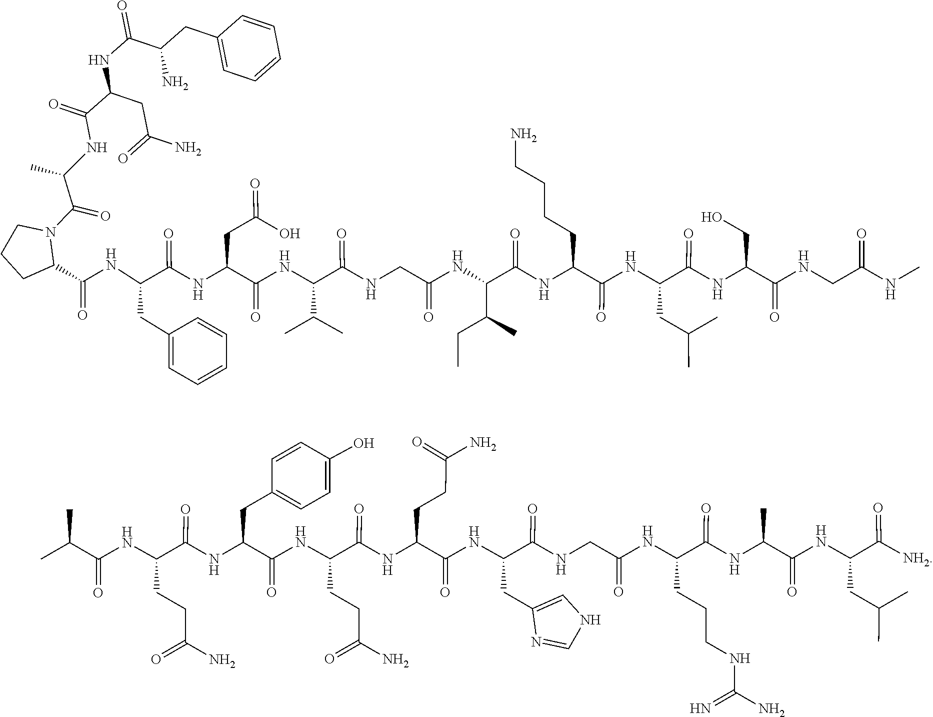

The agonist of the GPR39 receptor, AZ1395, has an IUPAC name of 3,4-bis-(2-imidazol-1-ylethoxy)-benzonitrile. AZ1395 has the following structure:

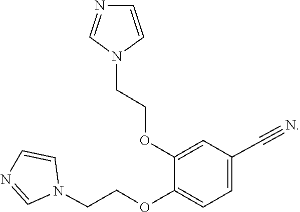 [SMILES: N#Cc1ccc(OCCn2ccnc2)c(OCCn2ccnc2)c1]

The agonist of the GPR39 receptor, AZ4237, has an IUPAC name of 6-[(4-chlorophenyl)methyl]-7-hydroxy-5-methyl-pyrazolo[1,5-a]pyrimidine-3-carboxylic acid. AZ4237 has the following structure:

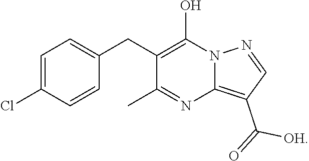 [SMILES: Cc1nc2c(C(=O)O)cnn2c(O)c1Cc1ccc(Cl)cc1]

The agonist of the GPR39 receptor, AZ7914, has an IUPAC name of 6-(3-chloro-2-fluoro-benzoyl)-2-(2-methylthiazol-4-yl)-3,5,7,8-tetrahydropyrido-[4,3-d]pyrimidin-4-one. AZ7914 has the following structure:

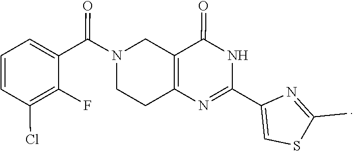 [SMILES: Cc1nc(-c2nc3c(c(=O)[nH]2)CN(C(=O)c2cccc(Cl)c2F)CC3)cs1]

Additional agonists of the GPR39 receptor include Compound 1 [PMID: 24900608] and Compound 15 [PMID: 25313322]. Compound 1 has the IUPAC name 6-[4-[(6-chloroimidazo[1,2-a]pyridin-2-yl)methyl]piperazin-1-yl]pyridine-3-carbonitrile. Compound 15 has the IUPAC name 4-N-[(2,4-dichlorophenyl)methyl]-2-N-methyl-6-pyridin-2-ylpyrimidine-2,4-diamine. For more information regarding Compound 1 and Compound 15, see guidetopharmacology.org/GRAC/LigandDisplayForward?ligandld=7800.

For additional examples and information of GPR39 receptor agonists, see U.S. Ser. No. 10/703,733, US20080015265, Frimurer et al., J. Med. Chem. 2017, 60, 886-898; Fjellstrom et al, doi.org/10.1371/journal.pone.0145849 and Brown et al., Novel GPR39 Agonists: Correlation of Binding Affinity Using Label-Free Back Scattering Interferometry with Potency in Functional Assays. The GPR39 receptor agonist compounds disclosed within these citations are specifically incorporated by reference herein.

Two subsets of purinergic receptors have been described; ligand-gated ionotropic P2X receptors, which induce Ca2+ influx; and metabotropic G-coupled P2Y receptors, which induce Ca2+efflux from the ER. Regarding enhancing thymic regeneration by targeting purinergic receptors, there are two clear mechanistic targets to promote thymus regeneration: (1) targeting cells that produce regenerative factors, such as BM P4 and IL23, that subsequently act on TECs to promote regeneration; and (2) to directly target TECs (by inducing the expression of a key TEC transcription factor FOXN1). Without being limited by theory, this disclosure describes that damage to the thymus induces a distinct alteration in cell death mechanisms, primarily in thymocytes which preferentially undergo pyroptotic cell death. As such, increased levels of multiple damage-associated molecular patterns (DAMPs) were identified in the thymus after damage (using sub-lethal total body irradiation as a damage model). This disclosure provides that some of these DAMPs, primarily ATP, can induce the production of regenerative factors in thymic endothelial and dendritic cells. The data also shows that ATP can induce FOXN1, a crucial transcription factor for thymus regeneration, in TECs. In vivo treatment with either ATP or a P2Y2 (a cell-surface purinergic receptor that is activated by ATP binding) agonist after damage leads to enhanced thymus regeneration and increased recovery of cTECs. Without being bound by theory, in vitro data suggests that the mechanism P2Y2 uses to induce FOXN1 in TECs is Ca2+ dependent. Thus, the current disclosure provides enhancing thymus regeneration by targeting and activating purinergic receptors (e.g., P2Y2, P2Y1, P2Y14, P2Y6, P2X7, P2X3, P2X4, P2X1, P2X6, P2X2, P2X5, P2Y4, P2Y11, P2Y12, P2Y13, and others) widely expressed on TECs.

The agonist of the P2Y2 purinergic receptor, MRS 2768, has a molecular formula of C₁₅H₁₆N₂Na₄O₁₈P4 and IUPAC name of tetrasodium; [[(2R,3S,4R,5R)-5-(2,4-dioxopyrimidin-1-yl)-3,4-dihydroxyoxolan-2-yl]methoxy-oxidophosphoryl] [oxido-[oxido(phenoxy)phosphoryl]oxyphos phoryl] phosphate. MRS 2768 has the following structure:

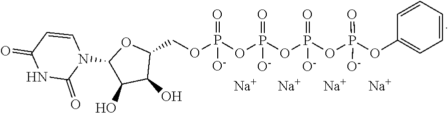 [SMILES: O=c1ccn([C@@H]2O[C@H](COP(=O)([O-])OP(=O)([O-])OP(=O)([O-])OP(=O)([O-])Oc3ccccc3)[C@@H](O)[C@H]2O)c(=O)[nH]1 ; [Na+] ; [Na+] ; [Na+] ; [Na+]]

Examples of additional agonists of the P2Y2 purinergic receptor include Denufosol and Diquafosol. Denufosol has a molecular formula of C₁₈H₂₇N₅O₂₁P₄ and IUPAC name of [[(2R,3S, 5R)-5-(4-amino-2-oxopyrimidin-1-yl)-3-hydroxyoxolan-2-yl]methoxy-hydroxyphosphoryl] [[[(2R,3S,4R,5R)-5-(2,4-dioxopyrimidin-1-yl)-3,4-dihydroxyoxolan-2-yl]methoxy-hydroxyphosphoryl]oxy-hydroxyphosphoryl] hydrogen phosphate. Denufosol has the following structure:

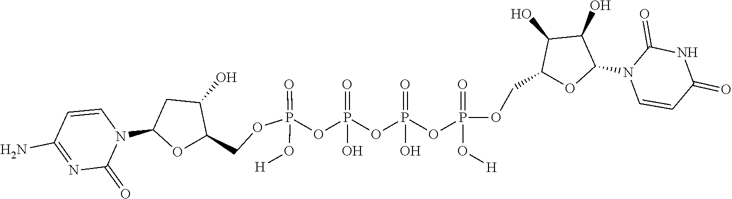 [SMILES: Nc1ccn([C@H]2C[C@H](O)[C@@H](COP(=O)(O)OP(=O)(O)OP(=O)(O)OP(=O)(O)OC[C@H]3O[C@@H](n4ccc(=O)[nH]c4=O)[C@H](O)[C@@H]3O)O2)c(=O)n1]

The agonist of the P2Y2 purinergic receptor, Diquafosol, also known as INS365, has a molecular formula of C₁₈H₂₆N₄O₂₃P₄ and IUPAC name of [[(2R,3S,4R,5R)-5-(2,4-dioxopyrimidin-1-yl)-3,4-dihydroxyoxolan-2-yl]methoxy-hydroxyphosphoryl] [[[(2R,3S,4R,5R)-5-(2,4-dioxopyrimidin-1-yl)-3,4-dihydroxyoxolan-2-yl]methoxy-hydroxyphosphoryl]oxy-hydroxyphosphoryl] hydrogen phosphate. Diquafosol has the following structure:

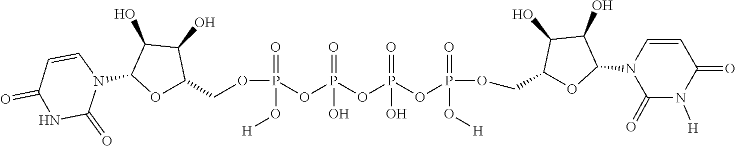 [SMILES: O=c1ccn([C@@H]2O[C@H](COP(=O)(O)OP(=O)(O)OP(=O)(O)OP(=O)(O)OC[C@@H]3O[C@H](n4ccc(=O)[nH]c4=O)[C@@H](O)[C@H]3O)[C@@H](O)[C@H]2O)c(=O)[nH]1]

For additional information regarding agonists of the P2Y2 purinergic receptor and functional derivatives thereof, see CN111253456, ES2553788, and WO2015165975. Additional agonists of the P2Y2 purinergic receptor and information regarding agonists described elsewhere herein include: ATP (PMID: 8564228, PMID:11754592, and PMID:12213051); 4-thio-UTP (PMID:16475938); 5BrUTP (PMID:8564228); Ap4A (PMID:711032 and PMID:1393282); MRS2698 (PMID:17302398); uridine triphosphate (UTP) (PMID:8564228 and PMID:11754592); UTPγS (PMID:8564228); 2-thioUTP (PMID:17125260); and PSB1114 (PMID:17125260, PMID:17088057, and PMID:21417463).

In addition to P2Y2 receptor agonists, agonists for other purinergic receptors can also be used. Agonists for the P2Y1 receptor include MRS2170 with an IUPAC name of 2′ Deoxy 6 N Methyladenosine 3′,5′ Bisphosphate; MRS2267; and MRS2279. Additional exemplary agonists for P2Y1 include [3H]2MeSADP; MRS2365; 2-CI-ADP(α-BH3); compound 3a [PMID: 22873688]; ADPβS; Ap3a; Ap5a; 2′,3′-ddATP; dATPαS; ATPyS; 2MeSATP; ATP; ADP; and [35S]ADPβS.

Agonists for the P2Y14 receptor include uridine diphosphate (UDP), UDP-glucose, UDP-galactose, UDP-glucuronic acid, UDP-N-acetylglucosamine, and other UDP-sugars. For additional information see Xu et al., Bioorganic & Medicinal Chemistry, 2018, 26(2), 366-375. Additional exemplary agonists for P2Y14 include α.β-methylene-2-thio-UDP; MRS4183; MRS2905; 2-thio-UDP; MRS2802; and MRS2690.

Exemplary agonists for P2Y6 include MRS2957, MRS2693, MRS4162, UDP-β-S, 3-phenacyl-UDP, INS48823, Up3U, uridine diphosphate, and MRS2782. Exemplary antagonists for P2Y6 include reactive blue-2, PPADS, suramin, MRS2578, MRS2578, MRS2567, and TIM-38.

Exemplary agonists for P2X7 include BzATP and ATP.

Exemplary agonists for P2X3 include ATP, BzATP, and αβ-meATP.

Exemplary agonists for P2X4 include ATP and αβ-meATP.

Exemplary agonists for P2X1 include ATP, BzATP, αβ-meATP, and L-βγ-meATP.

An exemplary agonist for P2X6, P2X2, and P2X5 includes ATP.

Exemplary agonists for P2Y4 include ATP and uridine triphosphate.

Exemplary agonists for P2Y11 include AR-C67085, NF546, ATPy-S, uridine triphosphate, BzATP, dATP, ATP, ADPβS, 2MeSATP, NAADP, and NAD.

Exemplary agonists for P2Y12 include 2MeSADP, ADP, ADPβ, and [3H]2MeSADP.

Exemplary agonists for P2Y13 include [33P]2MeSADP, 2MeSADP, 2MeSATP, ADP, ADPβS, and ATP.

In particular embodiments, the compositions and methods additionally promote thymic regeneration by reducing or inhibiting nucleotide-binding oligomerization domain-containing protein 2 (NOD2), Rho GTPases, and/or microRNA 29c (miR29c). Inhibition of NOD2, Rho GTPases, and/or miR29c can upregulate regenerative molecules, such as interleukin (IL)-22, IL-23, and bone morphogenetic protein 4 (BMP4).

Inhibitors of NOD2 can include ponatinib, regorafenib, and gefitinib which are multikinase inhibitors that target RIP2 kinase which forms a complex with NOD2 (Canning, et al. Chem Biol., 2015, 22, 1174-1184; Jakopin, Med Chem., 2014, 57, 6897-6918). The structures of ponatinib, regorafenib, and gefitinib respectively include:

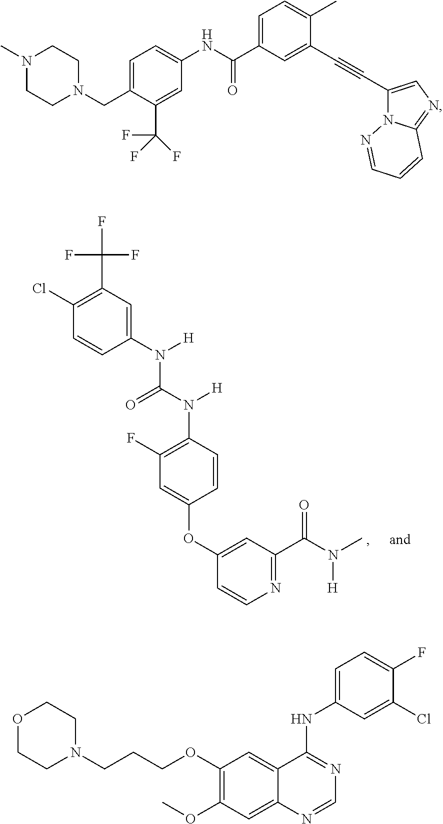 [SMILES: Cc1ccc(C(=O)Nc2ccc(CN3CCN(C)CC3)c(C(F)(F)F)c2)cc1C#Cc1cnc2cccnn12 ; CNC(=O)c1cc(Oc2ccc(NC(=O)Nc3ccc(Cl)c(C(F)(F)F)c3)c(F)c2)ccn1 ; COc1cc2ncnc(Nc3ccc(F)c(Cl)c3)c2cc1OCCCN1CCOCC1]

Additional inhibitors of NOD2 can include natural or endogenous compounds such as: Curcumin, a polyphenol from the plant Curcuma longa; sesquiterpene lactones such as parthenolide and helenalin; Pseudopterosins, such as pseudoterosin A, which are diterpenoid glycosides of marine origin; and polyunsaturated fatty acids such as docosahexaenoic acid (DHA) and eicosapentaenoic acid (EPA). These structures are shown below:

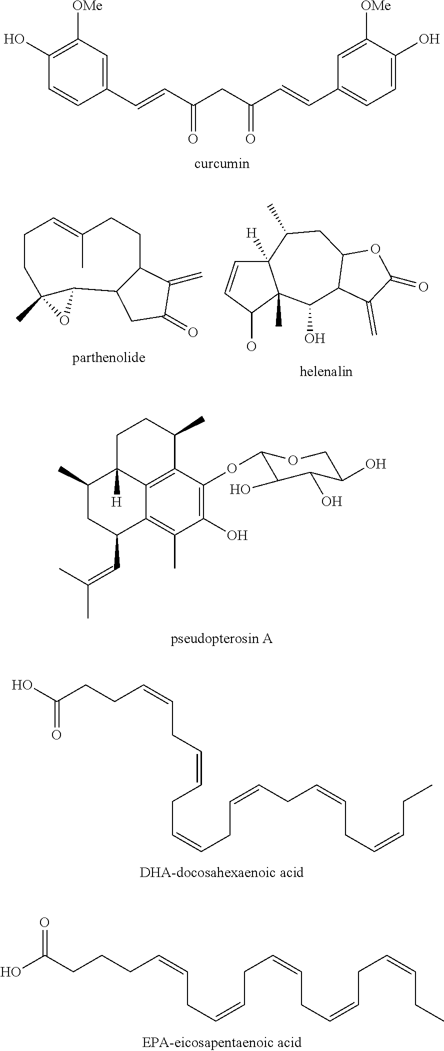

Inhibitors of NOD2 can further include benzimidazole diamides (e.g. GSK669 and GSK717), representative structures of which are shown below:

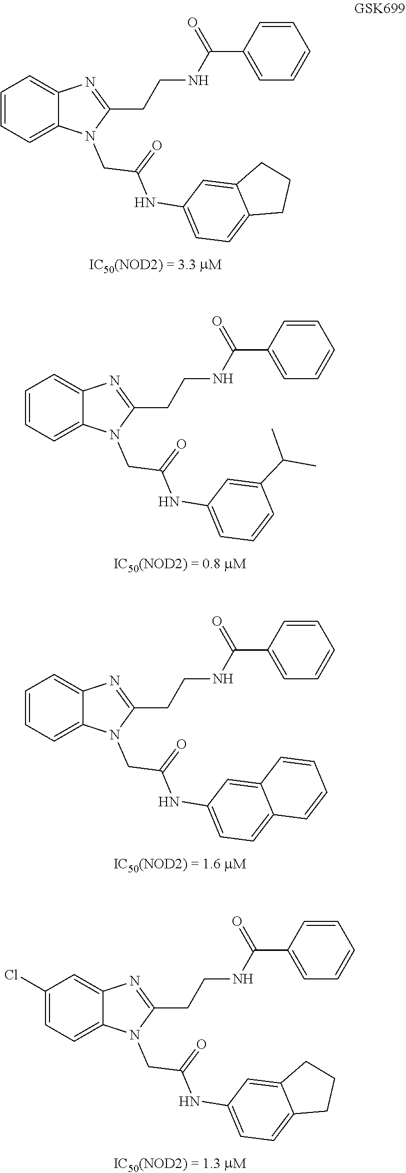 [SMILES: O=C(Cn1c(CCNC(=O)c2ccccc2)nc2ccccc21)Nc1ccc2c(c1)CCC2 ; CC(C)c1cccc(NC(=O)Cn2c(CCNC(=O)c3ccccc3)nc3ccccc32)c1 ; O=C(Cn1c(CCNC(=O)c2ccccc2)nc2ccccc21)Nc1ccc2ccccc2c1 ; O=C(Cn1c(CCNC(=O)c2ccccc2)nc2cc(Cl)ccc21)Nc1ccc2c(c1)CCC2]

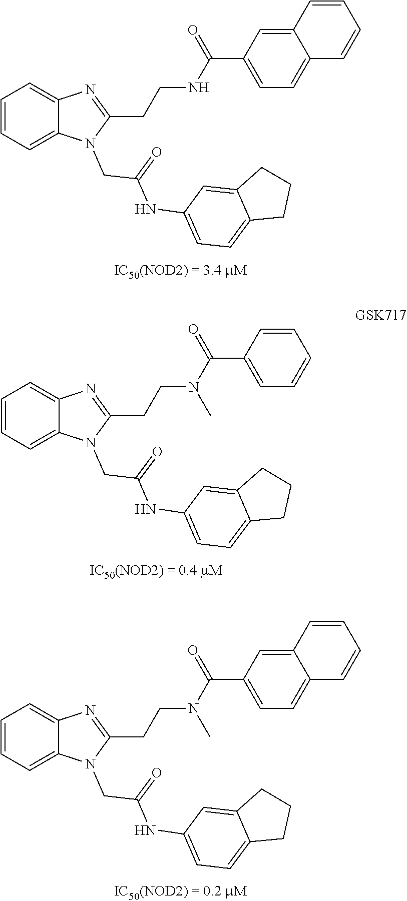 [SMILES: O=C(Cn1c(CCNC(=O)c2ccc3ccccc3c2)nc2ccccc21)Nc1ccc2c(c1)CCC2 ; CN(CCc1nc2ccccc2n1CC(=O)Nc1ccc2c(c1)CCC2)C(=O)c1ccccc1 ; CN(CCc1nc2ccccc2n1CC(=O)Nc1ccc2c(c1)CCC2)C(=O)c1ccc2ccccc2c1]

GSK717 NOD2 Signaling Inhibitor II is commercially available from Millipore Sigma (Cat#533718, Burlington, Mass.).

Inhibitors of NOD2 can further include hydrophenalene-chromium complexes, representative structures of which are shown below:

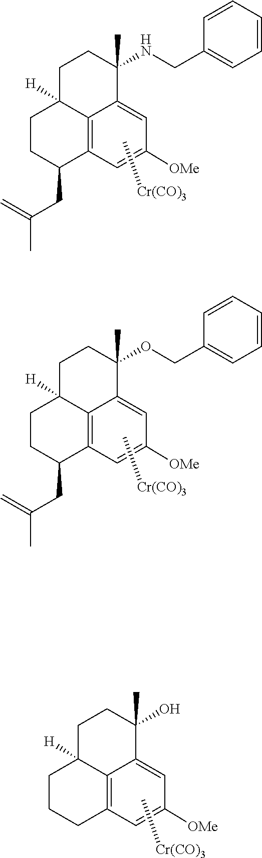

Additional inhibitors of NOD2 are disclosed in, for example, Jakopin Z (2014) Journal of Medicinal Chemistry 57(16): 6897-6918 and Rickard D J et al. (2013) PLoS ONE 8(8): e69619.

Inhibitors of Rho GTPases, such as RhoA and Rac1 can include: isoflavones such as genistein, daidzein, and glycitein (Seok et al. (2008) Journal of Pharmacology and Experimental Therapeutics 326(3): 991-998); 2-substituted quinoline (or quinoxaline) derivatives such as (E)-3-(3-(ethyl(quinolin-2-yl)amino)phenyl)acrylic acid and (E)-3-(3-(butyl(quinolin-2-yl)amino) phenyl)acrylic acid (Ma et al. (2015) ChemMedChem 10(1): 193-206); C3 transferase covalently linked to a proprietary cell penetrating moiety via a disulfide bond (Cat #CT03, Cytoskeleton Inc., Denver, Colo.); BA-210 (Cethrin® (BioAxone BioSciences Inc., Cambridge, Mass.), a recombinant fusion protein composed of C3 enzyme (Lord-Fontaine et al. (2008) J Neurotrauma 25: 1309-1322); ZCL 278 or 2-(4-Bromo-2-chlorophenoxy)-N-[[[4-[[(4,6-dimethyl-2-pyrimidinyl)amino]sulfonyl]phenyl]amino] thioxomethyl] acetamide, Cdc42 inhibitor (Cat #4794, Tocris, Minneapolis, Minn.); Rhosin hydrochloride or D-Tryptophan (2E)-2-(6-quinoxalinylmethylene)hydrazide hydrochloride, Rho GTPase inhibitor (Cat #5003, Tocris, Minneapolis, Minn.; Shang et al. (2012) Chemistry & Biology 19: 699-710); ML 141 or 4-[4,5-Dihydro-5-(4-methoxyphenyl)-3-phenyl-1H-pyrazol-1-yl]benzenesulfonamide, selective inhibitor of Cdc42 Rho family GTPase (Cat #4266, Tocris, Minneapolis, Minn.; Hong et al. (2013) J Biol Chem 288(12): 8531-8543); CASIN, Cdc42 inhibitor (Florian et al. (2012) Cell Stem Cell 10: 520-530); p120 catenin, a RhoA inhibitor (Anastasiadis (2000) Nature Cell Biology 2: 637-644); MLS000532223, Rho family GTPase inhibitor (Surviladze et al. (2010) J Biomolecular Screening 15(1): 10-20); and MLS000573151, Cdc42 inhibitor (Surviladze et al. (2010), supra). Small molecule RhoA inhibitors are further disclosed in Deng et al. (2011) J Med Chem 54(13): 4508-4522.

Inhibitors of Rac GTPases can particularly include: EHT 1864 (54547-(Trifluoromethyl)quinolin-4-ylthio)pentyloxy)-2-(morpholinomethyl)-4H-pyran-4-one dihydrochloride, a potent inhibitor of Rac family GTPases including Rac1, Rac1b, Rac2, and Rac3 (Cat #3872, Tocris, Minneapolis, Minn.)); Rac1 Inhibitor W56 (MVDGKPVNLGLWDTAG, SEQ ID NO: 1), a peptide including residues 45-60 of the guanine nucleotide exchange factor (GEF) recognition/activation site of Rac1 that selectively inhibits Rac1 interaction with Rac1-specific GEFs TrioN, GEF-H1 and Tiam1 (Cat #2221, Tocris, Minneapolis, Minn.); NSC 23766 or N⁶-[2-[[4-(Diethylamino)-1-methylbutyl]amino]-6-methyl-4-pyrimidinyl]-2-methyl-4,6-quinolinediamine trihydrochloride, selective inhibitor of Rac1-GEF interaction (Cat #2161, Tocris, Minneapolis, Minn.; Gao et al. (2004) PNAS USA 101: 7618-7623); EHop 016 or N⁴-(9-Ethyl-9H-carbazol-3-yl)-N²-[3-(4-morpholinyl)propyl]-2,4-pyrimidinediamine, Rac inhibitor (Cat #6248, Tocris, Minneapolis, Minn.; Montalvo-Ortiz et al. (2012) J Biol Chem 287(16): 13228-13238); and 6-mercaptopurine (6-MP) and its derivative 6-thioguanosine-5′-triphosphate (6-T-GTP) (Marinkovic et al. (2014) J Immunol 192(9): 4370-4378).

In particular embodiments, miR29c refers to Accession No. MIMAT0000536 (UAGCACCAUUUGAAAUCGGUUA (SEQ ID NO: 2)). In particular embodiments, miR29c refers to Accession No. MIMAT0004632 (UGACCGAUUUCUCCUGGUGUUC (SEQ ID NO: 3)). In particular embodiments, miR29c refers to UAGCACCAUUUGAAAUCGGU (SEQ ID NO: 4). For additional information regarding miR29c, see, for example, WO2008154098; Lagos-Quintana et al., Curr Biol. 12:735-739(2002); Poy et al., Nature. 432:226-230(2004); Landgraf et al., Cell. 129:1401-1414(2007); Ahn et al., Mol Hum Reprod. 16:463-471(2010); and Chiang et al., Genes Dev. 24:992-1009(2010).

In particular embodiments, an inhibitor of miR29c includes an antisense compound targeted to miR29c. In particular embodiments, an inhibitor of miR29c includes a modified oligonucleotide having a nucleobase sequence that is complementary to miR29c or a precursor thereof. In particular embodiments, an inhibitor of miR29c can be mmu-miR-29c-5p (AUCUCUUACACAGGCUGACCGAUUUCUCCUGGUGUUCAGAGUCUGUUUUUGUCUAGCA CCAUUUGAAAUCGGUUAUGAUGUAGGGGGA (SEQ ID NO: 5)). Inhibitors of miR29c can also include other small molecules or compounds such as PPAR-γ agonists including pioglitazone; 15-deoxy-delta-12,14-PGJ₂; or a thiazolidinedione.

Compounds that activate GPR39 and/or compounds that activate a purinergic receptor (e.g., P2Y2), and optionally a compound that inhibits NOD2, Rho GTPases, and/or miR29c can be formulated into compositions for administration to subjects. Compositions include a compound or molecule that activates GPR39 or a purinergic receptor (e.g., P2Y2) and a pharmaceutically acceptable carrier. Compounds can also include pharmaceutically acceptable salts, tautomers, isomers, and prodrugs thereof.

Exemplary pharmaceutically acceptable salts include acetate, acid citrate, acid phosphate, ascorbate, benzenesulfonate, benzoate, besylate, bisulfate, bitartrate, bromide, chloride, citrate, ethanesulfonate, formate, fumarate, gentisinate, gluconate, glucaronate, glutamate, lactate, methanesulfonate, nitrate, iodide, isonicotinate, maleate, oleate, oxalate, p-toluenesulfonate, pamoate (i.e., 1,1′-methylene-bis-(2-hydroxy-3-naphthoate)), pantothenate, phosphate, saccharate, salicylate, succinate, sulfate, tannate and tartrate salts.

“Prodrugs” refer to compounds that can undergo biotransformation (e.g., either spontaneous or enzymatic) within a subject to release, or to convert (e.g., enzymatically, mechanically, electromagnetically, etc.) an active or more active form of a compound after administration. Prodrugs can be used to overcome issues associated with stability, toxicity, lack of specificity, or limited bioavailability and often offer advantages related to solubility, tissue compatibility, and/or delayed release (See e.g., Bundgard, Design of Prodrugs, pp. 7-9, 21-24, Elsevier, Amsterdam (1985); and Silverman, The Organic Chemistry of Drug Design and Drag Action, pp. 352-401, Academic Press, San Diego, Calif. (1992)).

Pharmaceutically acceptable carriers include those that do not produce significantly adverse, allergic or other untoward reactions that outweigh the benefit of administration, whether for research, prophylactic and/or therapeutic treatments. Exemplary pharmaceutically acceptable carriers and formulations are disclosed in Remington's Pharmaceutical Sciences, 18th Ed. Mack Printing Company, 1990. Moreover, compositions can be prepared to meet sterility, pyrogenicity, general safety and purity standards as required by United States FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants.

For injection, compositions can be made as aqueous solutions, such as in buffers such as Hanks' solution, Ringer's solution, or physiological saline. The solutions can contain formulatory agents such as suspending, stabilizing and/or dispersing agents. Alternatively, the composition can be in lyophilized and/or powder form for constitution with a suitable vehicle, e.g., sterile pyrogen-free water, before use.

For oral administration, the compositions can be made as tablets, pills, dragees, capsules, liquids, gels, syrups, slurries, suspensions and the like.

For administration by inhalation, compositions can be made as aerosol sprays from pressurized packs or a nebulizer, with the use of a suitable propellant, e.g., dichlorodifluoromethane, trichlorofluoromethane, dichlorotetrafluoroethane, carbon dioxide or other suitable gas.

Compositions can also be depot preparations. Such long acting compositions may be administered by, for example, implantation or injection. Thus, for example, compounds can be formulated with suitable polymeric or hydrophobic materials (for example as an emulsion in an acceptable oil) or ion exchange resins, or as sparingly soluble derivatives, for example, as sparingly soluble salts.

Methods disclosed herein include promoting thymic regeneration. In particular embodiments, thymic regeneration is promoted by activating GPR39 and/or a purinergic receptor (e.g., P2Y2). In particular embodiments, FOXN1, IL-22, IL-23 and/or BMP4 are up-regulated by activating GPR39 or a purinergic receptor (e.g., P2Y2).

Particular embodiments disclosed herein can include treating subjects. Subjects include humans, veterinary animals (dogs, cats, reptiles, birds, etc.) livestock (horses, cattle, goats, pigs, chickens, etc.) and research animals (monkeys, rats, mice, fish, etc.) with compositions disclosed herein. Treating subjects includes delivering therapeutically effective amounts. Therapeutically effective amounts include those that provide effective amounts, prophylactic treatments and/or therapeutic treatments.

An “effective amount” is the amount of a compound necessary to result in a desired physiological change in the subject. Effective amounts are often administered for research purposes. Effective amounts disclosed herein can cause a statistically-significant effect in an animal model or in vitro assay relevant to thymic function and/or regeneration.

A “prophylactic treatment” includes a treatment administered to a subject who does not display signs or symptoms of thymic damage or displays only early signs or symptoms of thymic damage such that treatment is administered for the purpose of diminishing or decreasing the risk of developing thymic damage further. Thus, a prophylactic treatment functions as a preventative treatment against thymic damage. In particular embodiments, prophylactic treatments reduce, delay, or prevent thymic damage.

A “therapeutic treatment” includes a treatment administered to a subject who displays symptoms or signs of thymic damage and is administered to the subject for the purpose of diminishing or eliminating those signs or symptoms of thymic damage. The therapeutic treatment can reduce, control, or eliminate the presence or activity of thymic damage and/or reduce control or eliminate side effects of thymic damage. In particular embodiments, therapeutic treatments reduce, delay, or prevent further thymic damage from occurring. In particular embodiments, therapeutic treatments lead to improved thymic function. In particular embodiments, therapeutic treatments lead to thymic regeneration. In particular embodiments, a therapeutic treatment results in an increase in T cells.

In particular embodiments, a therapeutic treatment ameliorates at least one symptom of a disorder associated with thymic insufficiency. In particular embodiments, a thymic insufficiency is evidenced by a reduced numbers of T cells, e.g., CD4+ T cells, and/or naive (CD45RA+CD62L+) T cells. In particular embodiments, a thymic insufficiency is evidenced by T cell levels that are persistently (e.g., over a period of weeks to months) below a threshold level, e.g., below 50, 100, 200, 300, 400, or 500 cells/mm³ of whole blood; less than 50 naive T cells/mm³; and/or naive T cells of less than 5% of total T cells by flow cytometry. Alternatively or in addition, thymic insufficiency can be diagnosed based on a low number of recent thymic emigrating T cells via PCR-based measurement of TCR-excision circles (e.g., as described in Geenen et al., (2003). J. Endocrinol. 176, 305-311).

In particular embodiments, administration of a therapeutically effective amount can result in increased thymic mass and increased levels of naive, newly developed T cells. In particular embodiments, a therapeutic treatment results in an increase in numbers of T cells, e.g., levels of CD4+ T cells, and/or levels of naive (CD45 RA⁺CD62L⁺) T cells, that are persistently (e.g., over a period of weeks to months) above a threshold level. The threshold level can be above 50, 100, 200, 300, 400, or 500 cells/mm³ of whole blood. In particular embodiments, treatments disclosed herein result in more than 50 naive T cells/mm³ and/or naive T cells that include more than 5% of total T cells by flow cytometry. Thus methods disclosed herein can include monitoring numbers of T cells, e.g., levels of CD4+ T cells, and/or levels of naive (CD45RA-t-CD62L+) T cells, or monitoring the numbers of recent thymic emigrating T cells via PCR-based measurement of T cell receptor rearrangement excision circles (Geenen et al., (2003). J. Endocrinol. 176, 305-311) and adjusting or continuing dosing until a threshold level is reached.

In particular embodiments, thymic insufficiency is associated with a chronic infection, such as a viral or bacterial infection. Over time, a therapeutic treatment can result in T cells recognizing the infectious agent causing the infection. In particular embodiments, a therapeutic treatment can result in an increase in the variety of epitopes recognized by the subject's T cells (i.e., a more diverse T cell repertoire). In particular embodiments, the infection is with Human Immunodeficiency Virus (HIV), hepatitis (e.g., Hepatitis C or Hepatitis B virus); subacute sclerosing panencephalitis (chronic measles encephalitis); chronic papovavirus encephalitis (progressive multifocal leukoencephalopathy); and/or Epstein-Barr virus infection.

In particular embodiments, the subject has been exposed to a toxin that affects thymic size or function, e.g., organotin compounds, glucocorticosteroids, 2,3,7,8-tetrachlorodibenzo-p-dioxin, or cyclosporine (see, e.g., Schuurman et al., int J Immunopharmacol. 1992 April; 14(3):369-75). In particular embodiments, the subject has cancer, and has been treated with a chemotherapeutic agent that is thymotoxic.

Toxicity or lesions in the thymus has been reported in the following cancer treatments: pre-bone marrow transplantation conditioning, chemotherapy, radiotherapy (Heng et al., Curr Opin Pharmacol 10(4):425-33, 2010): cisplatin (Rebillard et al., Oncogene. 27(51):6590-5, 2008); cyclophosphamide (CPA) (Zusman et al., In Vivo. 16(6):567-76, 2002); NAVELBINE® (Pierre Fabre Medicament Joint Stock Company, Boulogne, France) i.v. Vinorelbine (Su et al., Int J Pharm. 411(1-2): 188-96, 2011); nucleoside-based analogues (Belinsky et al., Cancer Res, 67(1):262-8, 2007); fractionated low-dose radiation (Pogribny et al., Mol Cancer Res. 3(10):553-61, 2005); recombinant human IL-2 (rhlL-2) (Lee et al., Regul Toxicol Pharmacol. 64(2):253-62, 2012); CP-31398 (N′-[2-[2-(4-methoxyphenyl)ethenyl]-4-quinazolmyl]-N, N-dimethyl-1, 3-propanediamine dihydrochloride), a styrylquinazoline that stabilizes the DNA binding conformation of p53 (Johnson et al., Toxicology. 289(2-3): 141-50, 2011); synthetic retinoic acid analog, 9-cis-UAB30 [(2E,4E,6Z,8E)-8-(3′,4′-dihydro-1 ‘(2′H)-naphthalen-1’-ylidene)-3,7-dimethyl-2,4,6-octatrienoic acid], which is used to treat breast cancer (Kapetanovic, Int J Toxicol. 29(2): 157-64, 2010); flavopiridol, a cyclin-dependent kinase inhibitor, in treating non-small lung cancer (Zveleil, IDrugs. 1(2):241-6, 1998); E-41B (ethyl-4-isothiocyanatobutanoate) (Tulinska et al., Toxicology 145(2-3):217-25, 2000); 5-fluorouracil (5-FU) and its prodrug 5′-deoxy-5-fluorouridine (5′-DFUR) (Ishikawa et al., Jpn J Cancer Res. 80(6):583-91, 1989); and cyclosporine A (Bennett, J Natl Cancer Inst. 75(5):925-36, 1985), among others.

In particular embodiments, the subject has or is at risk of developing an autoimmune disease associated with or as a result of having a reduced numbers of T cells, or of an aberrant T cell repertoire; see, e.g., Datta and Sarvetnick, (2009) Trends Immunol 30, 430-438; Gagnerault, et al., (2009) The Journal of Immunology 183, 4913-4920; Kaminitz, et al., (2010). J Autoimmun 35, 145-152; King, et al., (2004) Cell 117, 265-277; and Zou et al. (2008) Eur J Immunol 38, 986-994.

In particular embodiments, the subject has experienced trauma to the thymic region or has had a surgical procedure that impacted the size of the thymus, e.g., cardiothoracie surgery (e.g., in neonates; see, e.g., Eysteinsdottir et al., Clin Exp Immunol. 2004, 136(2): 349-355). In particular embodiments, the subject has undergone a thymectomy, e.g., to treat cancer, e.g., thymoma, or to treat myasthenia gravis (Manlula et al., Chest 2005; 128:3454-3460).

When necessary to distinguish between a treatment that promotes thymic function and one that promotes thymic regeneration, a treatment that promotes thymic regeneration increases thymic mass, increases the size of the thymus, and/or increases the number of naïve, newly-developed T cells, particularly after acute or chronic injury where there has been a depletion of thymic cells. These regenerative effects can support and promote thymic function. In certain examples, treatments that promote thymic function increase a number of T cells (for example, from below a threshold to above a threshold) without necessarily impacting the size or mass of the thymus. Many treatments serve both purposes and promote thymic regeneration and function.

For administration, therapeutically effective amounts (also referred to herein as doses) can be initially estimated based on results from in vitro assays and/or animal model studies. The actual dose amount administered to a particular subject can be determined by a physician, veterinarian or researcher taking into account parameters such as physical and physiological factors including target, body weight, severity of thymic damage, cause of thymic damage, stage of thymic damage, previous or concurrent therapeutic interventions, idiopathy of the subject and route of administration.

Useful doses can range from 0.01 to 500 μg/kg or from 0.01 to 500 mg/kg. Therapeutically effective amounts can be achieved by administering single or multiple doses during the course of a treatment regimen (e.g., daily, every other day, weekly, monthly, every 6 months, or yearly).

In particular embodiments, the methods described herein are employed in combination with one or more other treatment modalities, e.g., treatment modalities for the regeneration of the thymus or parts thereof, e.g., as described in Lynch, et al., (2009) Trends Immunol 30, 366-373. Exemplary methods include castration (Griffith et al., (2011) Aging Cell 11, 169-177); administration of keratinocyte growth factor (KGF; Min et al., (2007), Blood 109, 2529-2537); administration of ghrelin (Dixit et al., (2007). J Clin Invest 1 17, 2778-2790); administration of human growth hormone (Goya et al., (1992). Brain Behay. Immun. 6, 341-354); and administration of interleukin-22 (Dudakov et al., (2012). Science 336, 91-95) or BMP4 (US 20170292111). Thus, the methods can include administering a GPR29 receptor agonist, a purinergic (e.g., P2Y2) receptor agonist, a NOD2, Rho GTPase, and/or miR29c inhibitor in combination with KGF, ghrelin, human growth hormone, and/or IL-22. e.g., administered simultaneously, e.g., in the same or different pharmaceutical composition and at substantially the same time (e.g., within 30-60 minutes of each other), or administered sequentially, e.g., in one or more doses.

In particular embodiments, the methods also include transplanting thymic tissues into a subject, e.g., where the subject lacks a thymus altogether, e.g., due to genetic reasons, e.g., DiGeorge syndrome, or as a result of other causes including those listed above. In particular embodiments, allogeneic thymic tissue is transplanted, e.g., as described in Markert et al., Clin Immunol. 2010 May; 135(2):236-46; Markert et al., N Engl J Med, 1999 Oct 14:34 1 (16); 1180-9; Markert et al., Blood. 2004 Oct. 15; 104(8):2574-81; Markert et al., Blood. 2007 May 15; 109(10):4539-47; and Chinn and Markert, J Allergy Clin Immunol 2011 June; 127(6): 1351-5. In particular embodiments, the transplant includes a thymic epithelial cell, other thymic stromal cell or a stromal cell derived from another tissue such as skin, a hematopoietic thymic homing cell such as a common lymphoid progenitor cell, or a multipotent progenitor cell (see, e.g., Boehm and Bleul, Trends in Immunology 27(10):477-484 (2006); Dunon and Imhof, Blood, 81 (1): 1-8 (1993); Zlotoff and Bhandoola, Annals of the New York Academy of Sciences, 1217 (Year in Immunology): 122-138 (2011)). In particular embodiments immune suppressive treatments are also administered, as described in the above references.

### EXEMPLARY EMBODIMENTS

1. A method of treating a subject in need promoted thymic function including administering a therapeutically effective amount of a composition including a GPR39 receptor-activating compound and/or a purinergic receptor-activating compound thereby treating the subject in need of promoted thymic function.

2. The method of embodiment 1, wherein the treating promotes thymic regeneration.

3. The method of embodiment 1 or 2, wherein the subject is in need of promoted thymic function based on age or an immune-compromised status due to a treatment.

4. The method of embodiment 1 or 2, wherein the subject is in need of promoted thymic function due to infection, or a cancer treatment.

5. The method of any of embodiments 1-4, wherein the GPR39 receptor-activating compound includes TC-G 1008, LY2784544, GSK2636771, obestatin, AZ1395, AZ4237, AZ7914, AZ4502, AZ9309, AZ2097, Compound 1, and/or Compound 15.

6. The method of any of embodiments 1-5, wherein the purinergic receptor-activating compound activates a P2Y2 purinergic receptor, a P2Y1 purinergic receptor, a P2Y14 purinergic receptor, a P2Y6 purinergic receptor, a P2X7 purinergic receptor, a P2X3 purinergic receptor, a P2X4 purinergic receptor, a P2X1 purinergic receptor, a P2X2 purinergic receptor, a P2X5 purinergic receptor, a P2X6 purinergic receptor, a P2Y4 purinergic receptor, a P2Y11 purinergic receptor, a P2Y12 purinergic receptor, or a P2Y13 purinergic receptor.

7. The method of any of embodiments 1-6, wherein the purinergic receptor-activating compound activates the P2Y2 purinergic receptor.

8. The method of embodiment 7, wherein the purinergic receptor-activating compound that activates the P2Y2 purinergic receptor includes ATP, MRS 2768, MRS 2698, Denufosol, Diquafosol, 4-thio-UTP, 5BrUTP, Ap4A, uridine triphosphate (UTP), UTPγS, 2-thioUTP, and/or PSB1114.

9. The method of any of embodiments 1-7, wherein the purinergic receptor-activating compound activates the P2Y1 purinergic receptor.

10. The method of embodiment 9, wherein the purinergic receptor-activating compound that activates the P2Y1 purinergic receptor includes MRS2170, MRS2267, MRS2279, [3H]2MeSADP, MRS2365, 2-CI-ADP(α-BH3), compound 3a, ADPβS, Ap3a, Ap5a, 2′,3′-ddATP, dATPαS, ATPγS, 2MeSATP, ATP, ADP, and/or [35S]ADPβS.

11. The method of any of embodiments 1-10, wherein the purinergic receptor-activating compound activates the P2Y14 purinergic receptor.

12. The method of embodiment 11, wherein the purinergic receptor-activating compound that activates the P2Y14 purinergic receptor includes a uridine diphosphate (UDP), a UDP-sugar, an α.β-methylene-2-thio-UDP, an MRS4183, an MRS2905, a 2-thio-UDP, an MRS2802, and/or an MRS2690.

13. The method of embodiment 12, wherein the UDP-sugar includes UDP-glucose, UDP-galactose, UDP-glucoronic acid, and/or UDP-N-acetylglucosamine.

14. The method of any of embodiments 1-13, further including administering a therapeutically effective amount of a composition including an inhibitor selected from a NOD2 inhibitor, a Rho GTPase inhibitor, and/or an miR29c inhibitor.

15. The method of any of embodiment 14, wherein the NOD2 inhibitor includes ponatinib, regorafenib, gefitinib, curcumin, a sesquiterpene lactone, a pseudopterosin, a polyunsaturated fatty acid, a benzimidazole diamide, and/or a hydrophenalene-chromium complex.

16. The method of embodiment 15, wherein the sesquiterpene lactone includes parthenolide and/or helenalin.

17. The method of embodiment 15, wherein the pseudopterosin includes pseudopterosin A.

18. The method of embodiment 15, wherein the polyunsaturated fatty acid includes docosahexaenoic acid (DHA) and/or eicosapentaenoic acid (EPA).

19. The method of embodiment 15, wherein the benzimidazole diamide includes GSK669 and/or GSK717.

20. The method of any of embodiments 14-17, wherein the Rho GTPase inhibitor includes isoflavones, (E)-3-(3-(ethyl(quinolin-2-yl)amino)phenyl)acrylic acid, (E)-3-(3-(butyl(quinolin-2-yl)amino)phenyl)acrylic acid, C3 transferase, ZCL 278, Rhosin hydrochloride, ML 141, CASIN, p120 catenin, MLS000532223, and/or MLS000573151.

21. The method of any of embodiments 14-18, wherein the Rho GTPase inhibitor includes an RhoA inhibitor and/or a Rac1 inhibitor.

22. The method of embodiment 21, wherein the Rac1 inhibitor includes EHT 1864, Rac1 Inhibitor W56, NSC 23766, EHop 016, 6-mercaptopurine (6-MP), and/or 6-thioguanosine-5′-triphosphate (6-T-GTP).

23. The method of any of embodiments 14-22, wherein the miR29c inhibitor includes a complementary interfering RNA sequence.

24. The method of any of embodiments 14-23, wherein the miR29c inhibitor includes SEQ ID NO: 5.

25. The method of any of embodiments 14-24, wherein the miR29c inhibitor includes a PPAR-γ agonist.

26. The method of embodiment 25, wherein the PPAR-γ agonist includes pioglitazone; 15-deoxy-delta-12,14-PGJ₂; and/or thiazolidinedione.

27. A method of upregulating FOXN1, IL-22, IL-23, and/or BMP4 in a subject in need thereof including administering a therapeutically effective amount of a composition including a GPR39 receptor-activating compound and/or a purinergic receptor-activating compound to the subject thereby upregulating FOXN1, IL-22, IL-23, and/or BM P4 in the subject.

28. The method of embodiment 27, wherein the upregulating promotes thymic function (e.g., by promoting thymic regeneration).

29. The method of embodiment 28, wherein the subject is in need of upregulating to promote thymic function (e.g., by promoting thymic regeneration) based on age or an immune-compromised status due to a treatment.

30. The method of embodiment 2, wherein the subject is in need of upregulating to promote thymic function (e.g, by promoting thymic regeneration) due to infection, or a cancer treatment.

31. The method of any of embodiments 27-30, wherein the GPR39 receptor-activating compound includes TC-G 1008, LY2784544, GSK2636771, obestatin, AZ1395, AZ4237, AZ7914, AZ4502, AZ9309, AZ2097, Compound 1, and/or Compound 15.

32. The method of any of embodiments 27-31, wherein the purinergic receptor-activating compound activates a P2Y2 purinergic receptor, a P2Y1 purinergic receptor, a P2Y14 purinergic receptor, a P2Y6 purinergic receptor, a P2X7 purinergic receptor, a P2X3 purinergic receptor, a P2X4 purinergic receptor, a P2X1 purinergic receptor, a P2X2 purinergic receptor, a P2X5 purinergic receptor, a P2X6 purinergic receptor, a P2Y4 purinergic receptor, a P2Y11 purinergic receptor, a P2Y12 purinergic receptor, or a P2Y13 purinergic receptor.

33. The method of any of embodiments 27-32, wherein the purinergic receptor-activating compound activates the P2Y2 purinergic receptor.

34. The method of embodiment 32, wherein the purinergic receptor-activating compound that activates the P2Y2 purinergic receptor includes ATP, MRS 2768, MRS 2698, Denufosol, Diquafosol, 4-thio-UTP, 5BrUTP, Ap4A, uridine triphosphate (UTP), UTPγS, 2-thioUTP, and/or PSB1114.

35. The method of any of embodiments 27-34, wherein the purinergic receptor-activating compound activates the P2Y1 purinergic receptor.

36. The method of embodiment 35, wherein the purinergic receptor-activating compound activates that the P2Y1 purinergic receptor includes MRS2170, MRS2267, MRS2279, [3H]2MeSADP, MRS2365, 2-CI-ADP(α-BH3), compound 3a, ADPβS, Ap3a, Ap5a, 2′,3′-ddATP, dATPαS, ATPγS, 2MeSATP, ATP, ADP, and/or [35S]ADPβS.

37. The method of any of embodiments 27-36, wherein the purinergic receptor-activating compound activates the P2Y14 purinergic receptor.

38. The method of embodiment 37, wherein the purinergic receptor-activating compound that activates the P2Y14 purinergic receptor includes a uridine diphosphate (UDP), a UDP-sugar, an α.β-methylene-2-thio-UDP, an MRS4183, an MRS2905, a 2-thio-UDP, an MRS2802, and/or an MRS2690.

39. The method of embodiment 38, wherein the UDP-sugar includes UDP-glucose, UDP-galactose, UDP-glucoronic acid, and/or UDP-N-acetylglucosamine.

40. The method of any of embodiments 27-39, further including administering a therapeutically effective amount of a composition including an inhibitor including a NOD2 inhibitor, a Rho GTPase inhibitor, and/or an miR29c inhibitor.

41. The method of any of embodiment 40, wherein the NOD2 inhibitor includes ponatinib, regorafenib, gefitinib, curcumin, a sesquiterpene lactone, a pseudopterosin, a polyunsaturated fatty acid, a benzimidazole diamide, and/or a hydrophenalene-chromium complex.

42. The method of embodiment 41, wherein the sesquiterpene lactone includes parthenolide and/or helenalin.

43. The method of any of embodiment 41, wherein the pseudopterosin includes pseudopterosin A.

44. The method of embodiment 41, wherein the polyunsaturated fatty acid includes docosahexaenoic acid (DHA) and/or eicosapentaenoic acid (EPA).

45. The method of embodiment 41, wherein the benzimidazole diamide includes GSK669 and/or GSK717.

46. The method of any of embodiments 40-45, wherein the Rho GTPase inhibitor includes isoflavones, (E)-3-(3-(ethyl(quinolin-2-yl)amino)phenyl)acrylic acid, (E)-3-(3-(butyl(quinolin-2-yl)amino)phenyl)acrylic acid, C3 transferase, ZCL 278, Rhosin hydrochloride, ML 141, CASIN, p120 catenin, MLS000532223, and/or MLS000573151.

47. The method of any of embodiments 40-46, wherein the Rho GTPase inhibitor includes a RhoA inhibitor and/or a Rac1 inhibitor.

48. The method of embodiment 47, wherein the Rac1 inhibitor includes EHT 1864, Rac1 Inhibitor W56, NSC 23766, EHop 016, 6-mercaptopurine (6-MP), and/or 6-thioguanosine-5′-triphosphate (6-T-GTP).

49. The method of any of embodiments 40-48, wherein the miR29c inhibitor includes a complementary interfering RNA sequence.

50. The method of any of embodiments 40-49, wherein the miR29c inhibitor includes SEQ ID NO: 5.

51. The method of any of embodiments 40-50, wherein the miR29c inhibitor includes a PPAR-γ agonist.

52. The method of embodiment 51, wherein the PPAR-γ agonist includes pioglitazone; 15-deoxy-delta-12,14-PGJ₂; and/or thiazolidinedione.

53. A method of any of embodiments 27-52, wherein the upregulating promotes thymic function (e.g., by promoting thymic regeneration) in the subject.

54. A composition including a therapeutically effective amount of a GPR39 receptor-activating compound and/or a purinergic receptor-activating compound wherein the therapeutically effective amount(s) promotes thymic function (e.g., by promoting thymic regeneration).

55. The composition of embodiment 54, wherein the GPR39 receptor-activating compound includes TC-G 1008, LY2784544, GSK2636771, obestatin, AZ1395, AZ4237, AZ7914, AZ4502, AZ9309, AZ2097, Compound 1, and/or Compound 15.

56. The composition of embodiments 54 or 55, wherein the purinergic receptor-activating compound activates a P2Y2 purinergic receptor, a P2Y1 purinergic receptor, a P2Y14 purinergic receptor, a P2Y6 purinergic receptor, a P2X7 purinergic receptor, a P2X3 purinergic receptor, a P2X4 purinergic receptor, a P2X1 purinergic receptor, a P2X2 purinergic receptor, a P2X5 purinergic receptor, a P2X6 purinergic receptor, a P2Y4 purinergic receptor, a P2Y11 purinergic receptor, a P2Y12 purinergic receptor, or a P2Y13 purinergic receptor.

57. The composition of any of embodiments 54-56, wherein the purinergic receptor-activating compound activates the P2Y2 purinergic receptor.

58. The composition of 57, wherein the purinergic receptor-activating compound that activates the P2Y2 purinergic receptor includes ATP, MRS 2768, MRS 2698, Denufosol, Diquafosol, 4-thio-UTP, 5BrUTP, Ap4A, uridine triphosphate (UTP), UTPγS, 2-thioUTP, and/or PSB1114.

59. The composition of any of embodiments 54-58, wherein the purinergic receptor-activating compound activates the P2Y1 purinergic receptor

60. The composition of embodiment 59, wherein the purinergic receptor-activating compound that activates the P2Y1 purinergic receptor includes MRS2170, MRS2267, MRS2279, [3H]2MeSADP, MRS2365, 2-CI-ADP(α-BH3), compound 3a, ADPβS, Ap3a, Ap5a, 2′,3′-ddATP, dATPαS, ATPγS, 2MeSATP, ATP, ADP, and/or [35S]ADPβS.

61. The composition of any of embodiments 54-60, wherein the purinergic receptor-activating compound activates the P2Y14 purinergic receptor.

62. The composition of embodiment 61, wherein the purinergic receptor-activating compound that activates the P2Y14 purinergic receptor includes a uridine diphosphate (UDP), a UDP-sugar, an α.β-methylene-2-thio-UDP, an MRS4183, an MRS2905, a 2-thio-UDP, an MRS2802, and/or an MRS2690.

63. The composition of claim 62, wherein the UDP-sugar includes UDP-glucose, UDP-galactose, UDP-glucoronic acid, and/or UDP-N-acetylglucosamine.

64. The composition of any of embodiments 54-63, further including a therapeutically effective amount of an inhibitor including a NOD2 inhibitor, a Rho GTPase inhibitor, and/or an miR29c inhibitor wherein the therapeutically effective amount(s) promotes thymic regeneration.

65. The composition of embodiment 64, wherein the NOD2 inhibitor includes ponatinib, regorafenib, gefitinib, curcumin, a sesquiterpene lactone, a pseudopterosin, a polyunsaturated fatty acid, a benzimidazole diamide, and/or a hydrophenalene-chromium complex.

66. The composition of embodiment 65, wherein the sesquiterpene lactone includes parthenolide and/or helenalin.

67. The composition of embodiment 65, wherein the pseudopterosin includes pseudopterosin A.

68. The composition of embodiment 65, wherein the polyunsaturated fatty acid includes docosahexaenoic acid (DHA) and/or eicosapentaenoic acid (EPA).

69. The composition of embodiment 65, wherein the benzimidazole diamide includes GSK669 and/or GSK717.

70. The composition of any of embodiments 64-69, wherein the Rho GTPase inhibitor includes isoflavones, (E)-3-(3-(ethyl(quinolin-2-yl)amino)phenyl)acrylic acid, (E)-3-(3-(butyl(quinolin-2-yl)amino)phenyl)acrylic acid, C3 transferase, ZCL 278, Rhosin hydrochloride, ML 141, CASIN, p120 catenin, MLS000532223, and/or MLS000573151.

71. The composition of any of embodiments 64-70, wherein the Rho GTPase inhibitor includes a RhoA inhibitor and/or a Rac1 inhibitor.

72. The composition of embodiment 71, wherein the Rac1 inhibitor includes EHT 1864, Rac1 Inhibitor W56, NSC 23766, EHop 016, 6-mercaptopurine (6-MP), and/or 6-thioguanosine-5′-triphosphate (6-T-GTP).

73. The composition of any of embodiments 64-72, wherein the miR29c inhibitor includes a complementary interfering RNA sequence.

74. The composition of any of embodiments 64-73, wherein the miR29c inhibitor includes SEQ ID NO: 5.

75. The composition of any of embodiments 64-74, wherein the miR29c inhibitor includes a PPAR-γ agonist.

76. The composition of embodiment 75, wherein the PPAR-γ agonist includes pioglitazone; 15-deoxy-delta-12,14-PGJ₂; and/or thiazolidinedione.

77. The composition of any of embodiments claim 54-76, wherein the composition is labeled for use to treat a subject in need of promoted thymic function and/or thymic regeneration or at risk for needing promoted thymic function and/or thymic regeneration.

78. The composition of any of embodiment 77, wherein the subject is in need of promoted thymic function and/or thymic regeneration or is at risk of needing promoted thymic function and/or thymic regeneration based on age or an immune-compromised status due to a treatment.

79. The composition of embodiment 77 or 78, wherein the subject is in need of promoted thymic function and/or thymic regeneration or is at risk of needing promoted thymic function and/or thymic regeneration based on infection or a cancer treatment.

### Experimental Examples. Example 1. Activation of the Zinc-Sensing Receptor GPR39 Promotes T Cell Reconstitution after Hematopoietic Stem Cell Transplant

Abstract. Prolonged lymphopenia represents a major clinical problem after cytoreductive therapies such as chemotherapy and the conditioning required for hematopoietic stem cell transplant (HCT), contributing toward the risk of infections and malignant relapse. Restoration of T cell immunity is dependent on tissue regeneration in the thymus, the primary site of T cell development; although the capacity of the thymus to repair itself diminishes over lifespan. However, although boosting thymic function and T cell reconstitution is of considerable clinical importance, there are currently no approved therapies for treating lymphopenia. Here, Zinc (Zn) was found to be critically important for both normal T cell development as well as repair after acute damage. Accumulated Zn in thymocytes during development was released into the extracellular milieu after HCT conditioning, where it triggered regeneration by stimulating endothelial cell-production of BM P4 via the cell surface receptor GPR39. Dietary supplementation of Zn was sufficient to promote thymic function in a mouse model of allogeneic HCT, including enhancing the number of recent thymic emigrants in circulation; although direct targeting of GPR39 with a small molecule agonist enhanced thymic function without the need for prior Zn accumulation in thymocytes. Together, these findings not only indicate an important pathway underlying tissue regeneration, but also offer an innovative approach to treat lymphopenia in HCT recipients, among others requiring thymic support.

Introduction. The thymus, which is the primary site of T cell generation, is extremely sensitive to insult, but also has a remarkable capacity for endogenous repair (Granadier et al., Seminars in Immunopathology. 2021, 43:119-134; and Gruver and Sempowski, Journal of Leukocyte Biology. 2008, 84(4):915-923). However, even though there is likely continual thymic involution and regeneration in response to everyday insults like stress and infection, profound thymic damage such as that caused by common cancer therapies and the conditioning regimens used as part of hematopoietic stem cell transplantation (HCT) contributes to prolonged T cell depletion. Post-transplant lymphopenia precipitates high morbidity and mortality from opportunistic infections and likely facilitates malignant relapse (Small et al., Blood. 1999, 93(2):467-480; Maury et al., Br J Haematol. 2001, 115(3):630-641; Storek et al., Blood. 2001, 98(13):3505-3512; Storek et al., Am J Hematol. 1997, 54(2):131-138; Maraninchi et al., Lancet. 1987, 2(8552):175-178; Kinsella et al., Frontiers in Immunology. 2020, 11:1745; and Velardi et al., Nature Reviews Immunology. 2021, 21:277-291). At the present time there are no approved therapies to enhance post-transplant T cell reconstitution in recipients of HCT.

Zinc (Zn) is the second most abundant trace element in the body, capable of interacting with more than 300 proteins involved in almost all aspects of cell function (Kimura et al., Int J Mol Sci. 2016, 17(3):336; Cousins et al., J Biol Chem. 2006, 281(34):24085-24089; and Takagishi et al., Int J Mol Sci. 2017, 18(12)), including a well-established role in immune health (Honscheid et al., Metab Immune Disord Drug Targets. 2009, 9(2):132-144; Vallee et al., Physiol Rev. 1993, 73(1):79-118; and Hojyo et al., Journal of immunology research. 2016, 2016:6762343-6762343). Much of what is known about the effect of Zn on immune function comes from studies where dietary Zn has been deficient, either due to reduced intake due to malnourishment or via genetic means such as loss of function of ZIP4, a Zn transporter, which clinically leads to the condition Acrodermatitis enteropathica (Neldner et al., N Engl J Med. 1975, 292(17):879-882; Ogawa et al., J Immunol Res. 2018, 2018:5404093; Brummerstedt et al., Am J Pathol. 1977, 87(3):725-728; and Macdonald et al., Arch Dermatol. 2012, 148(8):961-963). In these settings of Zn deficiency, widespread immune effects can be seen, including defective B cell development, atrophy of the thymus, and disrupted T cell function (Honscheid et al., Metab Immune Disord Drug Targets. 2009, 9(2):132-144; Hojyo et al., Proc Natl Acad Sci USA. 2014, 111(32):11786-11791; Colomar-Carando et al., J Immunol. 2019, 202(2):441-450; Anzilotti et al., Nature Immunology. 2019, 20(3):350-361; Mitchell et al., 2006, 7(5-6):461-470; and Golden et al., The Lancet. 1977, 310(8047):1057-1059). However, while Zn deficiency (ZD) is known to lead to thymic involution, and supplementation with dietary Zn can ameliorate this phenotype (Hojyo et al., Journal of immunology research. 2016, 2016:6762343-6762343; and Wong et al., J Nutr. 2009, 139(7):1393-1397), the mechanisms by which Zn acts on thymic function is poorly understood.

Here, it is shown that not only is Zn important for the differentiation and development of thymocytes during T cell development; but its translocation after acute injury such as that caused by total body irradiation (TBI) can directly stimulate the production of BMP4 by endothelial cells (ECs), which has recently been found to be a critical pathway for endogenous thymic regeneration after acute injury (Wertheimer et al., Science Immunology. 2018, 3(19)). This putative role for Zn as a damage-associated molecular pattern (DAMP), was mediated by signaling through the cell surface Zn receptor, GPR39. Notably, while dietary zinc supplementation enhanced T cell reconstitution after allogeneic HCT, direct pharmacologic stimulation of GPR39 enhanced thymic regeneration and abrogated the need for a complex and prolonged Zn administration. The studies outlined here indicate an important pathway underlying tissue regeneration and also offers an innovative clinical approach to enhance T cell reconstitution in recipients of HCT, among others requiring thymic support.

Results. Zinc is crucial for steady-state T cell development and promoting regeneration after acute damage.

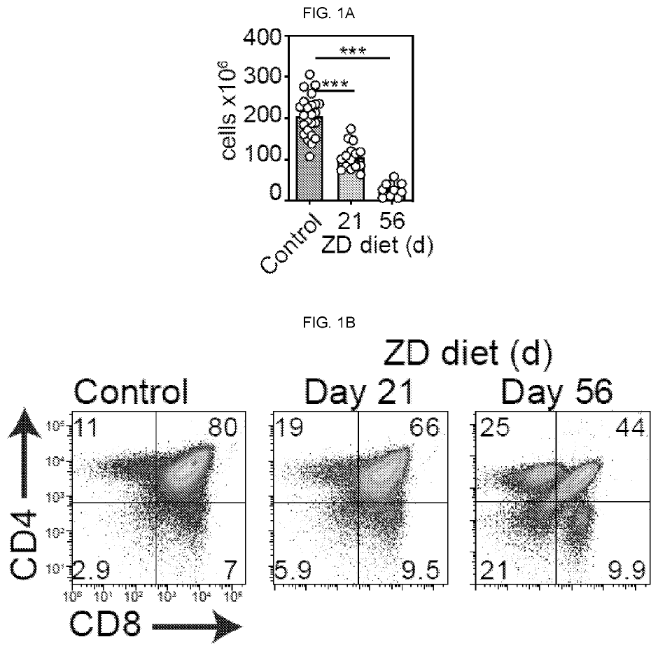

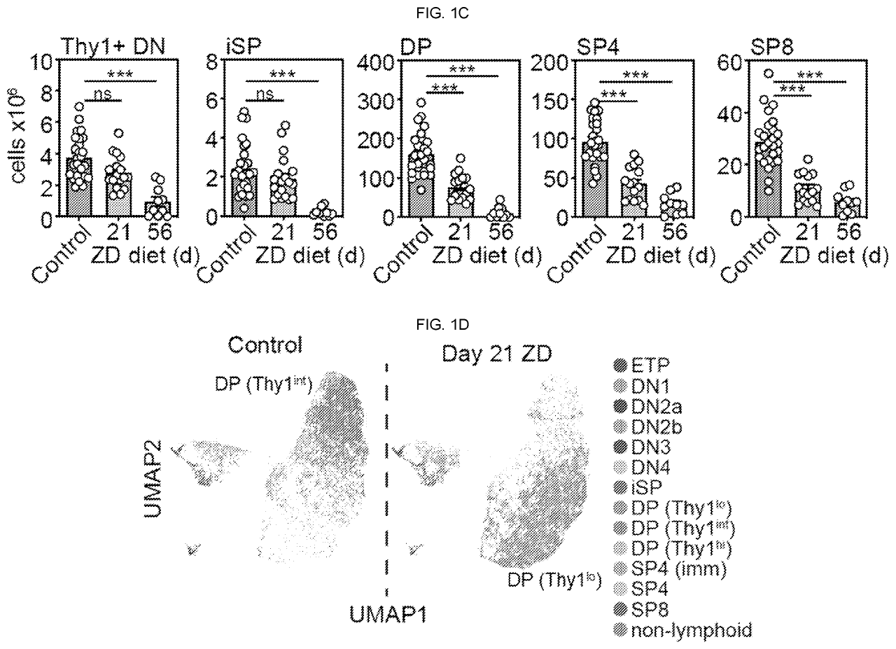

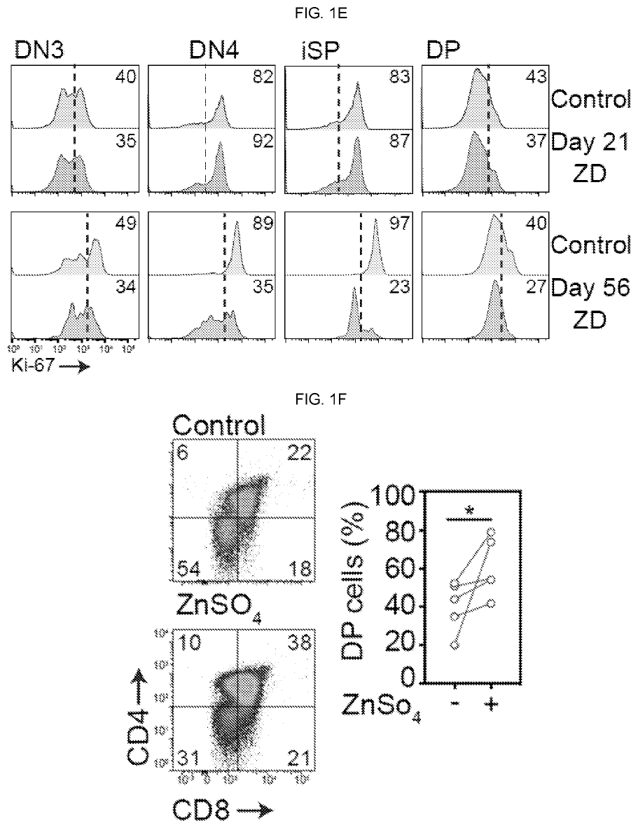

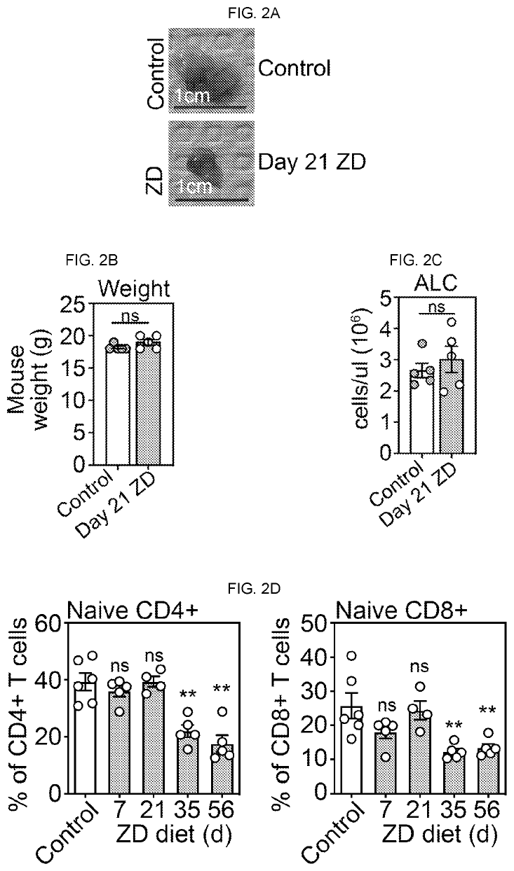

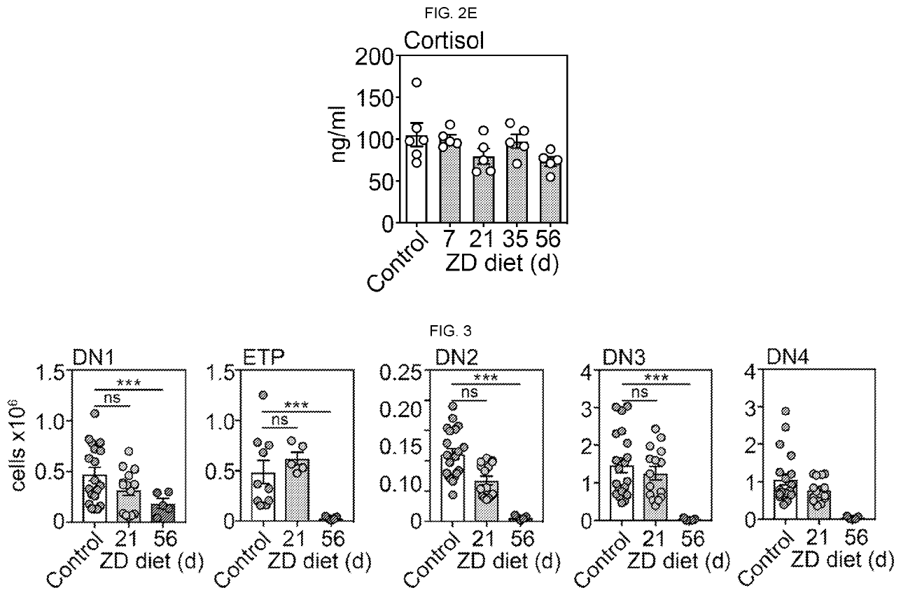

Modeling the effect of Zn on baseline thymic function, mice fed a Zn deficient (ZD) diet exhibited reduced thymic cellularity (FIGS. 1A and 2A) in as little as three weeks of ZD treatment when compared to age-matched mice that received normal chow. These effects were observed even when there were no gross phenotypes such as weight loss (FIG. 2B). Although Zn has previously been shown to affect peripheral T cells (Honscheid et al., Metab Immune Disord Drug Targets. 2009, 9(2):132-144; Colomar-Carando et al., J Immunol. 2019, 202(2):441-450; Bogale et al., Nutr Metab Insights. 2015, 8:7-14; and Coto et al., Proc Natl Acad Sci USA. 1992, 89(16):7752-7756), there was no effect of the ZD treatment on absolute lymphocyte count at 21 days (FIG. 2C), with significant decrease of naïve T cells seen from 5 weeks of ZD diet (FIG. 2D). Importantly, levels of cortisol, a stress hormone with considerable negative effects on the thymus (Gruver and Sempowski, Journal of Leukocyte Biology. 2008, 84(4):915-923), was consistent throughout the experiment (FIG. 2E). Cell depletion was not uniform amongst developing T cells as at day 21 there was a significant decrease only in double positive (DP) and single positive CD4+ and CD8+(SP4 and SP8) cells, but no change in the earlier double negative (DN), early thymic progenitors (ETP), or intermediate Single Positive (iSP) thymocytes (FIGS. 1B, 10, and 3). Within DP thymocytes, there appeared to be a block after ZD treatment that was marked by different level of expression of Thy1 (FIG. 1D), and a corresponding decline in proliferation (FIG. 1E). By 56 days after ZD, all thymocyte subsets were depleted with reduced proliferation (FIG. 1E). To confirm the importance of Zn on T cell maturation, lineage-negative bone marrow (BM) isolated from C57BL/6 mice were co-cultured on the OP9-DLL1 system, which is able to support T cell development in vitro (Schmitt et al., Nat Immunol. 2004, 5(4):410-417; and Holmes et al., Cold Spring Harb Protoc. 2009, 2009(2):pdb prot5156). Compared to control, BM cultured in media with ZnSO₄ showed more robust production of DP (FIG. 1F).

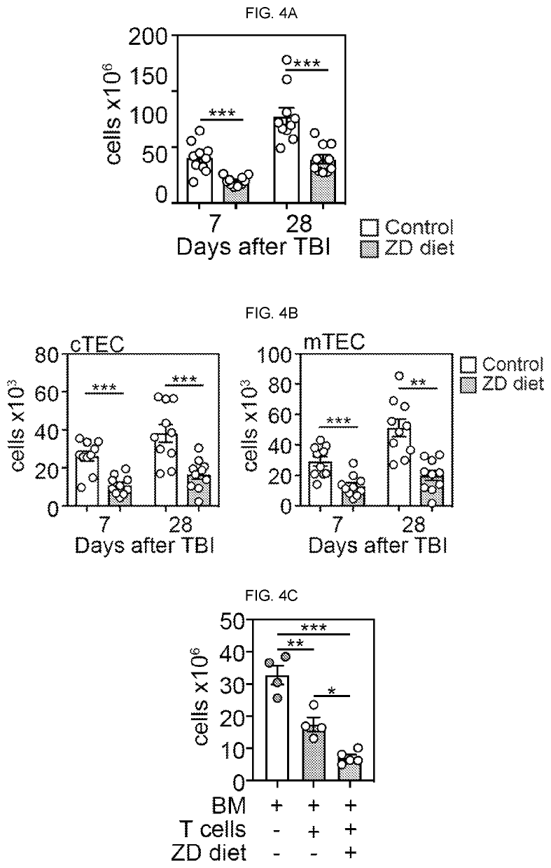

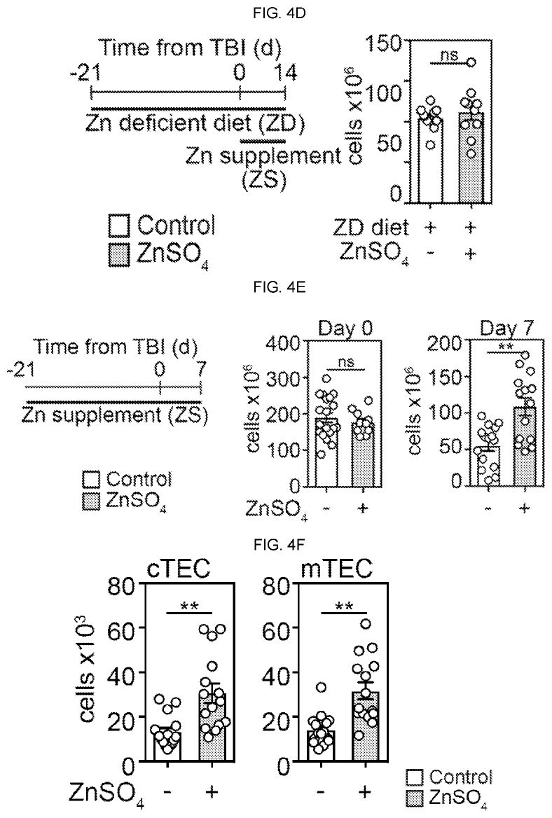

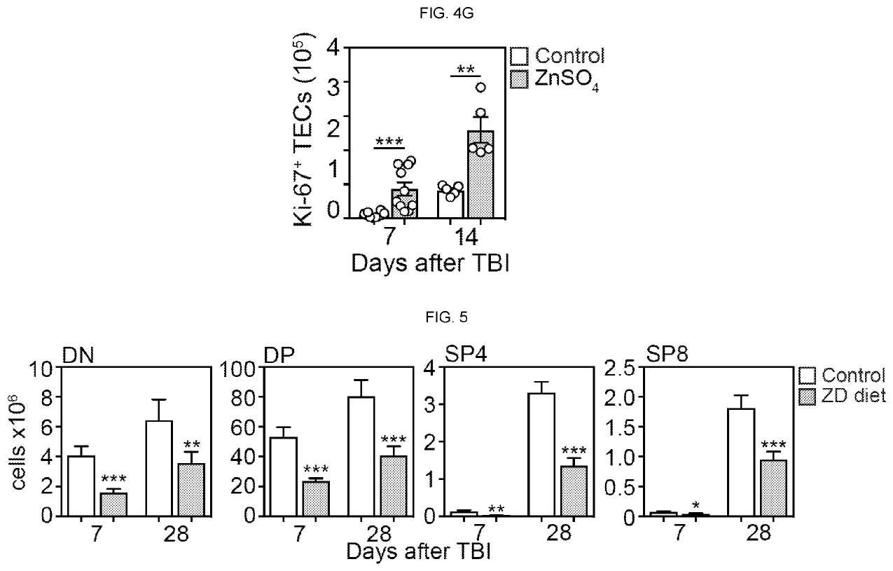

Perhaps unsurprisingly given its importance for maintaining thymopoiesis, mice that had been on a ZD diet exhibited poorer regeneration following a sublethal dose of total body irradiation (SL-TBI, 550cGy) (FIG. 4A), which was reflected amongst all developing thymocytes (FIG. 5) and supporting thymic epithelial cell (TEC) subsets (FIG. 4B). The thymus is extremely sensitive to graft versus host disease (GVHD), even in situations where GVHD may not be detected in classic target organs such as skin, gut or liver (Dudakov et al., Blood. 2017, 130(7):933-942; Hassan et al., Blood. 2015, 125(17):2593-2595; and Krenger et al., Semin Immunopathol. 2008, 30(4):439-456). Surprisingly, it was found that ZD could have an impact on thymic function even in mice with significant GVHD (FIG. 4C). Importantly, the thymic effects of a ZD diet could be ameliorated by supplementation of drinking water with Zn sulfate (300 mg/Kg/day) beginning on the day of irradiation (FIG. 4D). These findings demonstrate that even a short-term reduction in Zn intake has a detrimental impact on thymopoiesis, and the transition from DN to DP stage of T cell development, as well as post-damage thymic reconstitution.

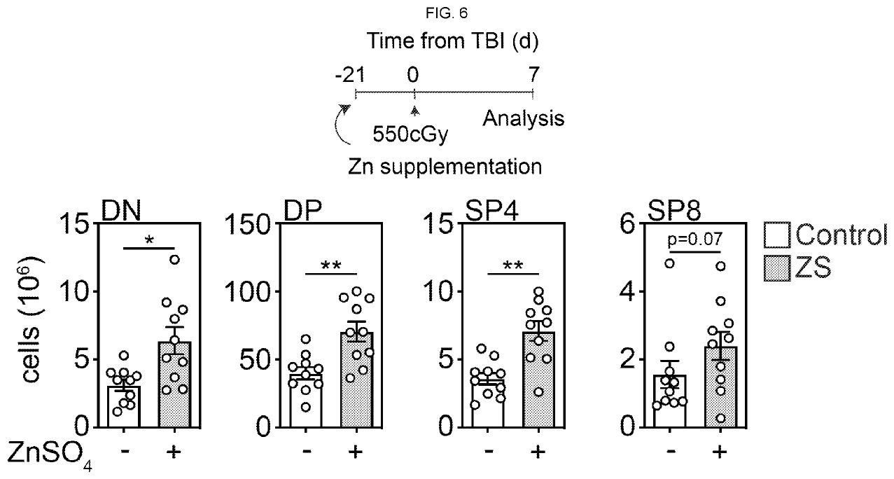

Since thymic function at both baseline and after damage was so sensitive to Zn availability, it was hypothesized that dietary Zn supplementation could improve thymic reconstitution after acute insult. Mice were put on Zn supplementation (ZS) (300 mg/Kg/day/mouse of ZnSO₄ monohydrate (in drinking water) (Wong et al., J Nutr. 2009, 139(7):1393-1397) for three weeks prior to SL-TBI and maintained for 7 days after. Although no difference in baseline thymic cellularity was observed between control and ZS mice after three weeks of treatment, mice that received ZS treatment exhibited improved reconstitution after SL-TBI (FIG. 4E), reflected by individual thymocyte populations (FIG. 6) and within TEC subsets (FIG. 4F). Both the absolute number and the proportion of proliferating TECs, measured by the expression of Ki-67, were higher in the thymuses from mice that received Zn supplementation (FIG. 4G).

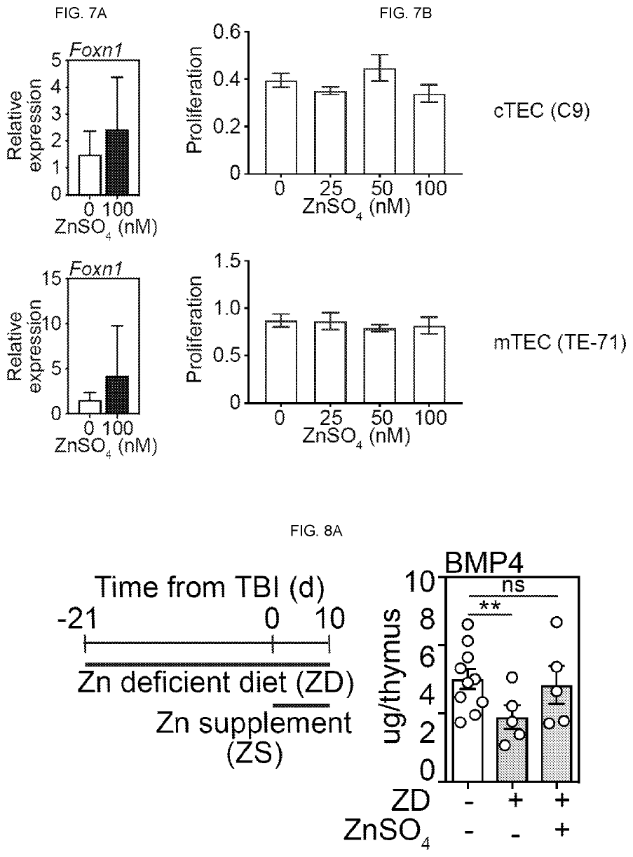

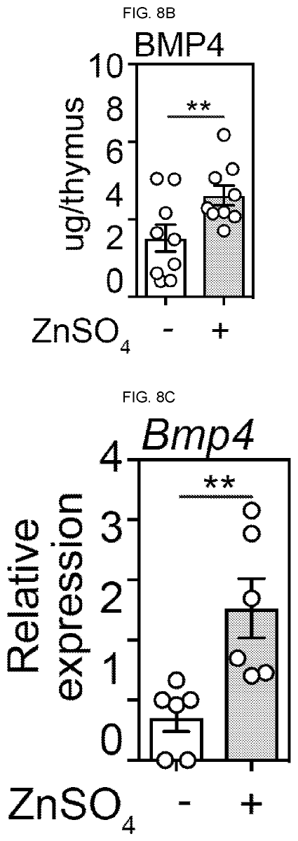

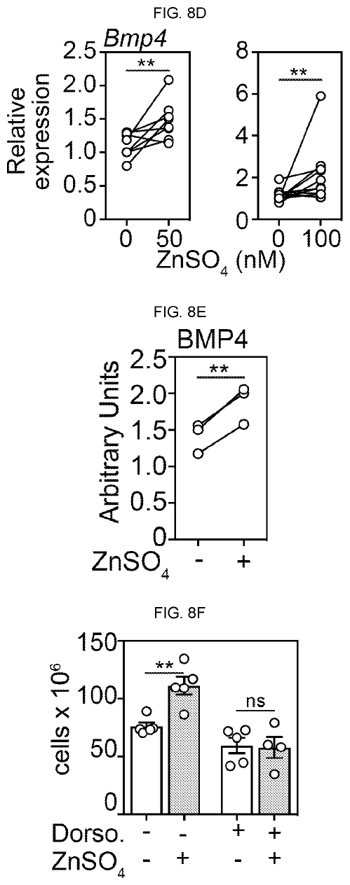

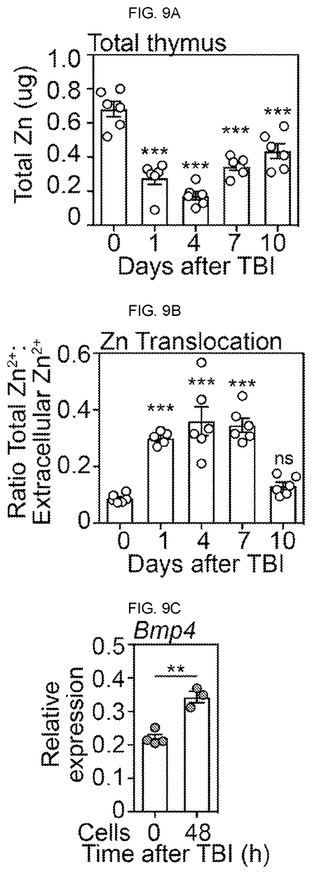

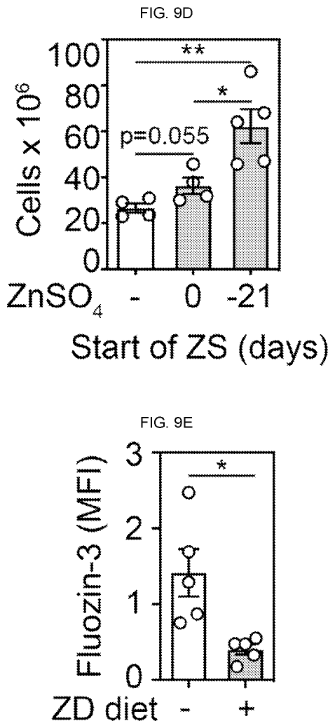

Zinc stimulates the production of BMP4 by thymic endothelial cells. Given the increased proliferation of TECs after ZS, the direct effect of ZnSO₄ was tested on proliferation of mouse cortical (C9) and medullary (TE-71) TEC cell lines. Observations did not show any direct effect of Zn on TEC proliferation, or their ability to express key thymopoietic transcription factors such as FOXN1 (FIGS. 7A and 7B). One mechanism by which TECs are induced to proliferate is via BMP4 stimulation, which is produced by ECs in response to damage and can mediate thymic repair by stimulating TEC regeneration (Wertheimer et al., Science Immunology. 2018, 3(19); and Barsanti et al., Eur J Immunol. 2017, 47(2):291-304). Interestingly, there is a body of work demonstrating that Zn plays an important role in vascular integrity and EC response to stress (Schulkens et al., J Vasc Res. 2014, 51(3):231-238; Hershfinkel et al., Int J Mol Sci. 2018, 19(2); and Fujie et al., J Toxicol Sci. 2016, 41(2):217-224). To determine if BMP4 could be a mediator of the effect of Zn in thymic regeneration, the level of BMP4 was first assessed in the thymus of mice that had received either a ZD diet for three weeks before TBI (and throughout the study) or mice that had received a ZD diet but had also been given ZS in drinking water. Assessing the expression of BMP4 by ELISA at day 10, a timepoint at the peak of expression after damage (Wertheimer et al., Science Immunology. 2018, 3(19)), it was found that mice that had received a ZD diet had significantly reduced levels of BMP4 in the thymus, but dietary supplementation of Zn restored BMP4 levels (FIG. 8A). Consistent with these findings, mice that had been given ZS without prior ZD had increased levels of BMP4 (FIG. 8B), and purified ECs from ZS-treated mice exhibited increased expression of Bmp4 measured by qPCR (FIG. 8C). Together, these findings suggest that Zn is not only involved in thymocyte maturation, but also in overall thymic regeneration by stimulating the production of BMP4 from EC.

To mechanistically interrogate the direct effect of Zn on thymic ECs, the Akt pathway was constitutively activated using the prosurvival adenoviral gene E4ORF1, which allows ECs from multiple tissues, including the thymus, to be propagated and manipulated ex vivo while maintaining their phenotype and vascular tube formation for functional manipulation and in vitro modeling of regenerative pathways (Wertheimer et al., Science Immunology. 2018, 3(19); Seandel et al., Proc Natl Acad Sci USA. 2008, 105(49):19288-19293; and Kinsella et al., bioRxiv. 2020, [Preprint](Aug. 31, 2020 [cited Aug. 23, 2021)): DOI: 2020.2008.2031.275834). Using this approach, it was found that ECs showed a dose-dependent increase in the transcription of Bmp4 by qPCR after 24 hours of exposure to exogenous Zn (FIG. 8D). This finding was confirmed at the protein level after 48 hours of exposure to Zn (FIG. 8E). Consistent with the hypothesis that Zn is involved in the in vivo pathway of BMP4 production, treatment with the pan-BMP-receptor inhibitor dorsomorphin dihydrochloride abrogated the effect of ZS on thymic regeneration (FIG. 8F).

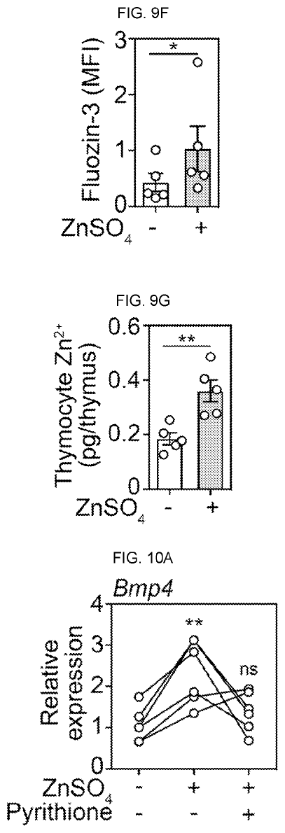

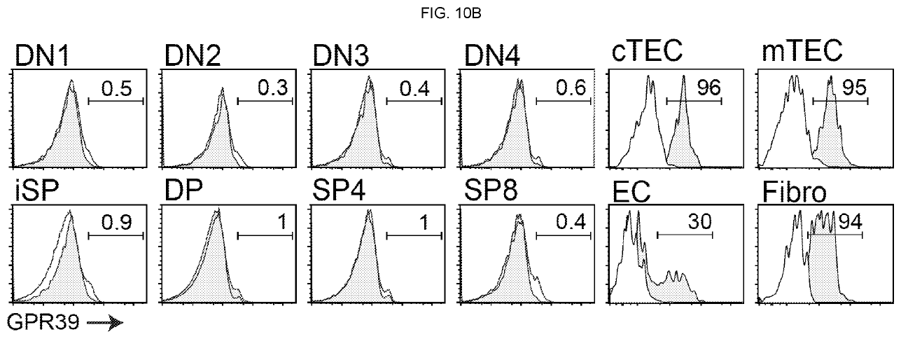

Extracellular translocation of Zn after acute damage stimulates production of BMP4 by ECs. To clarify how Zn mechanistically contributes to endogenous thymic regeneration, changes in Zn levels were first measured in otherwise untreated wild-type (WT) mice after TBI by inductively coupled plasma mass spectrometry (ICP-MS). Although the total amount of Zn in whole tissue lysates (both intracellular and extracellular compartments) decreased after damage, following the same trend as thymic cellularity (FIG. 9A) (Wertheimer et al., Science Immunology. 2018, 3(19)), when extracellular Zn (using supernatants) was assessed as a function of total Zn, a significant translocation of Zn from intracellular to extracellular space after damage was found (FIG. 9B).

To functionally assess this finding, exECs were co-cultured with supernatants isolated from ZS-treated mice at day 0 and 48 h after TBI. Increased expression of Bmp4 was found in ECs co-cultured with supernatant from thymuses harvested 2 days after TBI (FIG. 9C). Given that a better effect was observed if ZS is begun several weeks before TBI (FIG. 9D), it was hypothesized that thymocytes, which require Zn for their maturation, accumulate Zn during ZS which allows for increased bioavailability of extracellular Zn after damage and the triggering of regenerative responses in ECs. Consistent with this hypothesis, thymocytes isolated from mice given a ZD diet exhibited significantly lower levels of Zn (FIG. 9E) and mice given ZS in their drinking water showed significantly increased levels of intracellular Zn (FIGS. 9F and 9G).

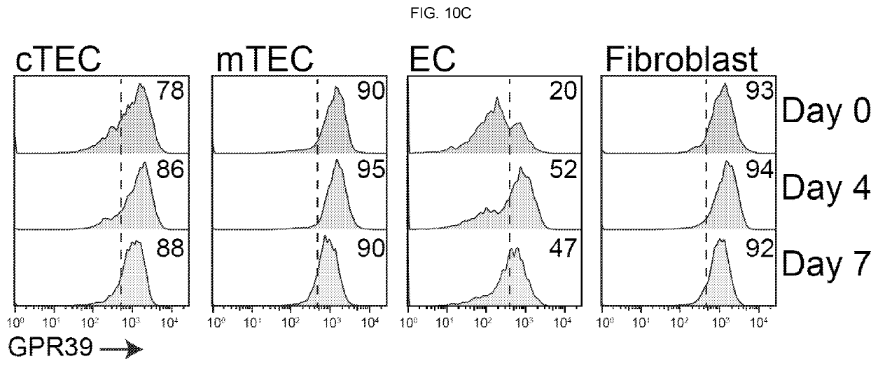

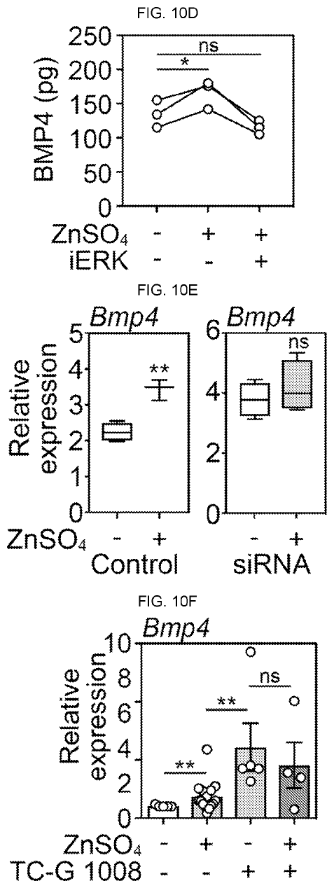

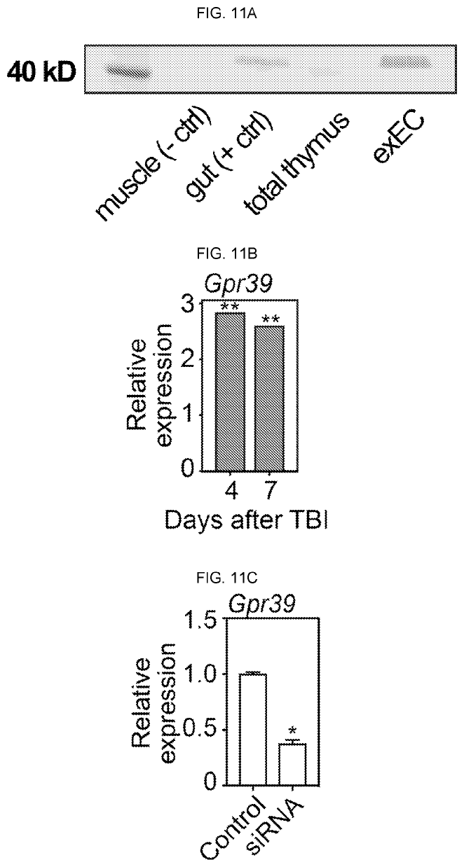

Zn signals though GPR39 on ECs to stimulate production of BMP4. There are two main modalities by which Zn can mediate its effect on cells, influx (and efflux) using the ZIP (and ZnT) ion channels (Prasad, J Am Coll Nutr. 2009, 28(3):257-265; and Kasana et al., J Trace Elem Med Biol. 2015, 29:47-62); or via the cell surface Zn-sending G-protein coupled receptor, GPR39 (Xu et al., Eur J Pharmacol. 2019, 858:172451; and Zhu et al., Am J Physiol Cell Physiol. 2018, 314(4):C404-C414). To identify the putative mode action for Zn, intracellular Zn concentrations of exECs were selectively increased by treatment with the Zn ionophore sodium pyrithione. An increase of Bmp4 expression was not observed after treatment with pyrithione (FIG. 10A), suggesting that binding to a surface receptor is more likely than through Zn internalization. GPR39 could not be detected on thymocyte populations; however, significant expression was found on non-hematopoietic stromal cells such as TECs, fibroblasts, and ECs (FIGS. 10B and 11A). Notably, while there was no change in expression within TECs or fibroblasts after damage, an increase in expression of GPR39 was found on ECs (FIGS. 10C and 11B), suggesting their potential to respond to extracellular Zn after damage is increased, and consistent with reports demonstrating that EC function can be regulated by GPR39 signaling (Xu et al., Eur J Pharmacol. 2019, 858:172451; and Zhu et al., Am J Physiol Cell Physiol. 2018, 314(4):C404-C414). GPR39 acts by translating extracellular Zn signals into release of intracellular second messengers such as ERK and calcium release (Hershfinkel et al., Int J Mol Sci. 2018, 19(2); and Sunuwar et al., Philos Trans R Soc Lond B Biol Sci. 2016, 371(1700)). Consistent with this, stimulation of exECs with Zn led to phosphorylation of ERK1/2 (FIG. 10D), and when ERK was blocked with the inhibitor FR180204 prior to Zn stimulation, BMP4 production was abrogated (FIG. 10D). Importantly, demonstrating the functional importance of GPR39 for EC-mediated regeneration, Zn-mediated production of Bmp4 was abrogated in exECs after silencing of Gpr39 expression (FIGS. 10E and 11C). Stimulation of exEC with the selective GPR39 agonist TC-G 1008 induced expression of Bmp4, greater than Zn alone (FIG. 10F).

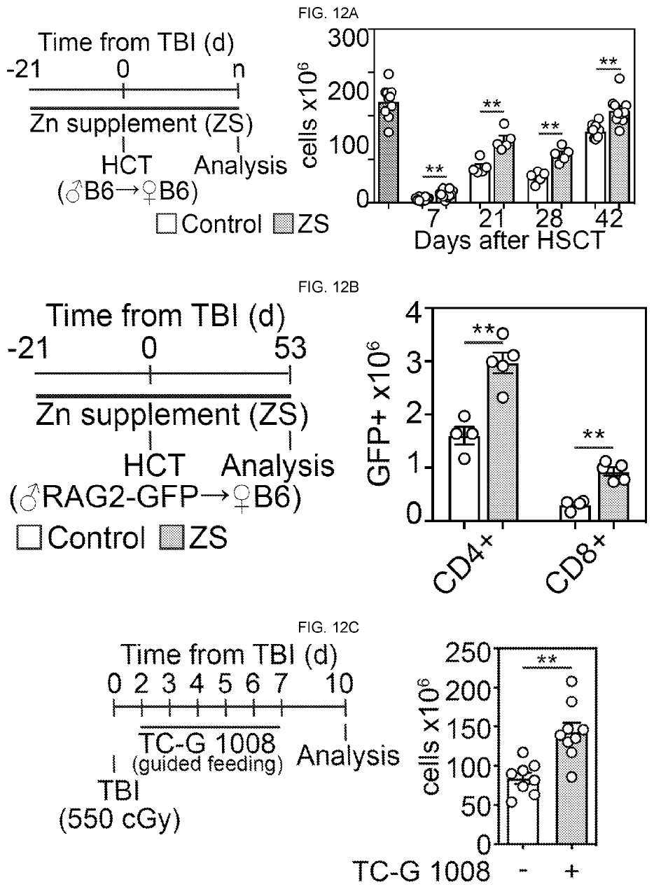

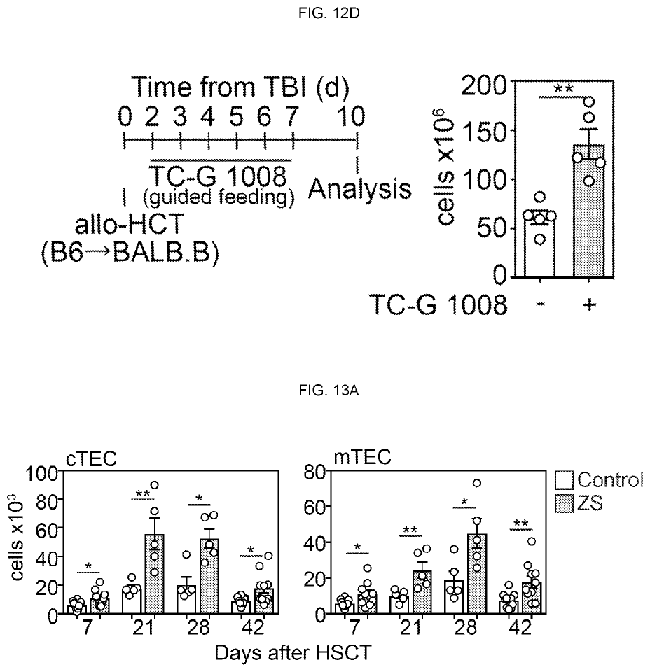

Activation of GPR39 signaling promotes T cell reconstitution after hematopoietic stem cell transplantation. Thymic regeneration is a particular challenge after the myeloablative conditioning required for successful HCT (Clave et al., Leukemia. 2012, 26(8):1886-1888). It was found that dietary ZS promoted thymic reconstitution in a minor-antigen mismatched model of murine T-depleted (TCD) allogeneic (allo)-HCT (where any effects mediated by GVHD can be excluded) (FIG. 12A). Increased cellularity was observed in all developing thymocyte subsets and TEC subsets (FIGS. 13A and 13B). To track the export of T cells from the thymus, TCD allo-HCT was performed where donors expressed green fluorescent protein (GFP) under the control of RAG2, which allows for the detection of cells recently exported from the thymus (referred to as recent thymic emigrants (RTEs) (Monroe et al., Immunity. 1999, 11(2):201-212; and Alves et al., J Immunol. 2010, 184(11):5949-5953). In both peripheral blood and spleen, mice that received ZS showed higher levels of GFP+CD4+ and CD8+ lymphocytes after HCT (FIG. 12B).

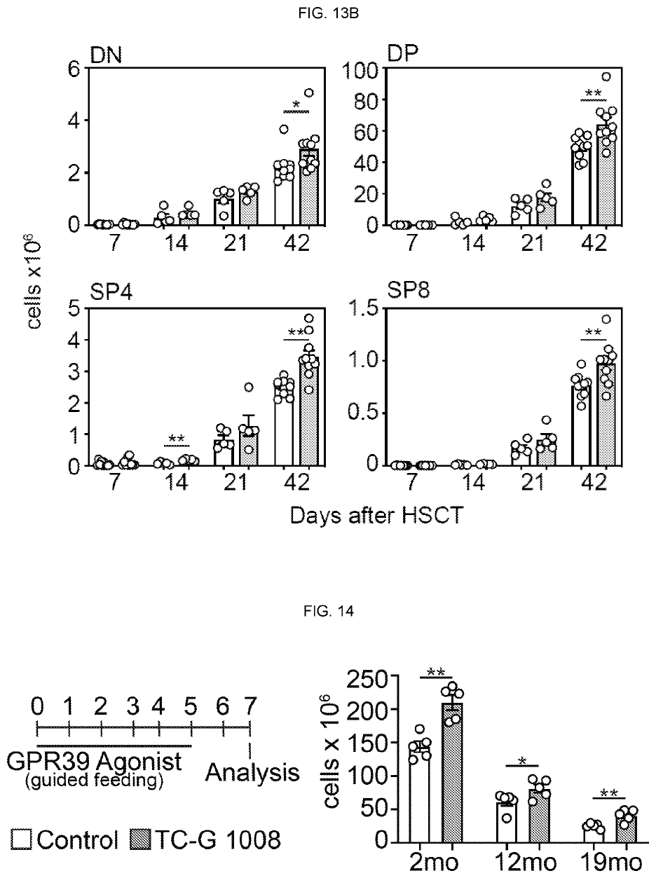

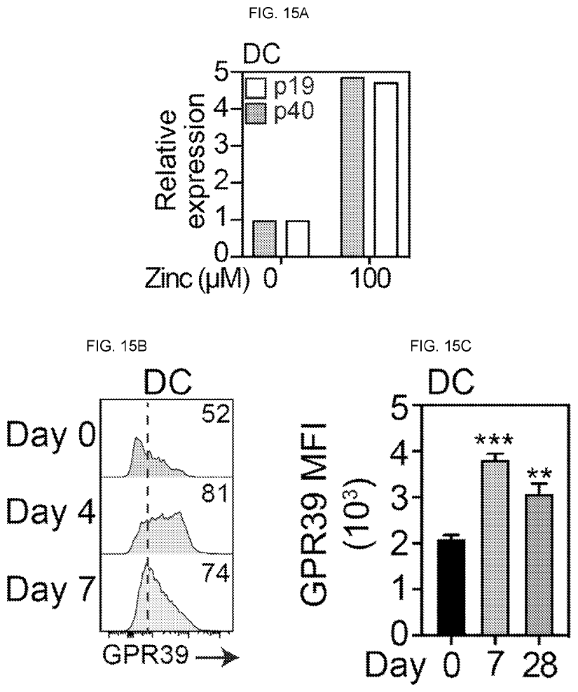

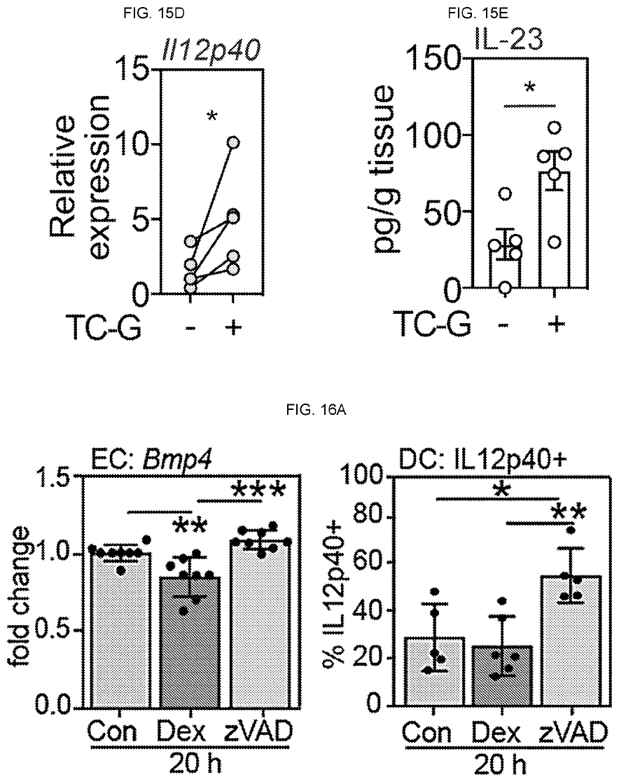

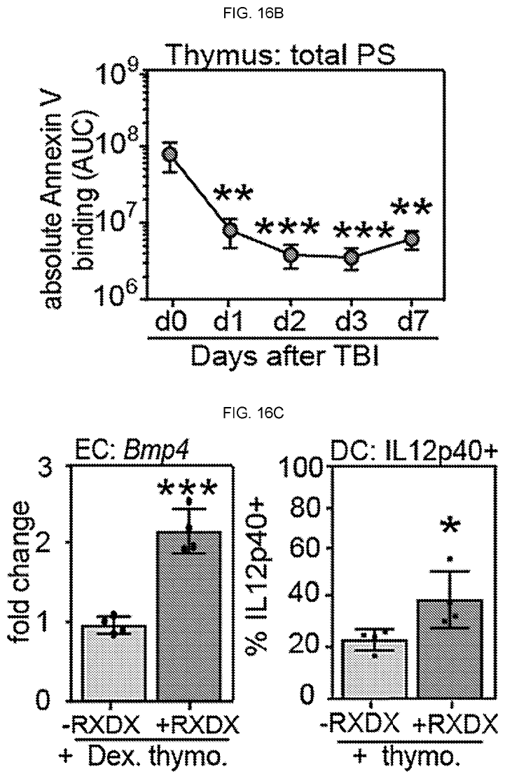

The complicated mechanism by which dietary Zn supplementation promotes thymic regeneration involves prolonged treatment before HCT for thymocytes to accumulate Zn to be released after injury; thereby allowing signaling through GPR39 in regeneration-initiating ECs. To test if directly stimulating GPR39 could abrogate this lead time, mice were treated with the GPR39 agonist TC-G 1008 (Peukert et al., ACS Med Chem Lett. 2014, 5(10):1114-1118). Importantly, using this approach it could be shown that mice treated with TC-G 1008 showed significantly improved thymic function in models of both SL-TBI (FIG. 12C) in mice given a TCD allo-HCT across multiple minor histocompatibility antigens (FIG. 12D). Taken together, these data suggest that improved thymic regeneration caused by Zn signaling can help immune reconstitution after HCT by increasing the production of thymic-derived naïve T cells. Additionally, this pathway can be pharmacologically targeted by stimulating GPR39 signaling. Furthermore, mice aged 2 months, 12 months, or 19 months treated with TC-G 1008 show significantly improved thymus cellularity compared to their untreated controls (FIG. 14).

Discussion Alterations in Zn uptake, retention, sequestration, or secretion can quickly lead to ZD and affect Zn-dependent functions in virtually all tissues, and in particular in the immune system, including thymic involution (Neldner et al., N Engl J Med. 1975, 292(17):879-882; Mitchell et al., 2006, 7(5-6):461-470; and Golden et al., The Lancet. 1977, 310(8047):1057-1059). This shows that even short-term zinc deprivation in young animals has a profound impact on thymic function, before effects on peripheral immune cells can be detected. ZD reduced the replicative ability of thymocytes, especially in the transition to DP thymocytes. Although the specific mechanism by which Zn is acting on thymocyte development is unclear, there is evidence that many Zn-finger transcriptional factors that heavily depend on general Zn availability are crucial for T cell development (Reed et al., Genes Immun. 2013, 14(1):7-12; Han et al., Elife. 2014, 3:e03549; and Moore et al., Proc Natl Acad Sci USA. 2003, 100(7):3883-3888). In this data, intracellular Zn levels in thymocytes responded rapidly to changes in systemic Zn availability; with lower levels of Zn in thymocytes under ZD and higher levels after ZS. This is consistent with the notion that T cells actively internalize Zn during activation and replication through the expression of Zn importers such as ZIP6 (Colomar-Carando et al., J Immunol. 2019, 202(2):441-450). Notably, the changes observed in thymic function preceded the other classic signs of ZD, such as weight loss and skin and fur changes, confirming the sensitivity of thymopoiesis to Zn level; although the contribution of systemic effects cannot be completely ruled out (King et al., J Nutr. 2002, 132(5):974-979).

Thymic regeneration is a complex process, in which the cytokines produced from damage-resistant cells, such as IL-22 from innate lymphoid cells (ILC), IL-23 from dendritic cells (DC), and BMP4 from endothelial cells (EC), stimulate TECs to proliferate and mediate broader thymic repair (Wertheimer et al., Science Immunology. 2018, 3(19); and Dudakov et al., Science. 2012, 336(6077):91-95). BMP4 is a member of bone morphogenic proteins, a family of peptides involved in embryogenesis and homeostasis of many tissues (Miyazono et al., Cytokine Growth Factor Rev. 2005, 16(3):251-263), including thymic organogenesis and maintenance of Foxn1 expression in TECs (Barsanti et al., Eur J Immunol. 2017, 47(2):291-304; Patel et al., Gene Expr Patterns. 2006, 6(8):794-799; and Gordon et al., Dev Biol. 2010, 339(1):141-154). Findings show that modulating levels of Zn in the thymus using either ZD or ZS had a concomitant effect on BMP4 expression. Furthermore, stimulation of thymic ECs with ZnSO₄ directly induced the production of BMP4 in a GPR39-dependent manner; and the administration of a BMP-receptor inhibitor abrogated the effect of ZS on thymic repair. Notably, a similar role for Zn release into the extracellular space after acute damage has been demonstrated to be involved in tissue repair in tissues such as skin and gut (Ogawa et al., J Immunol Res. 2018, 2018:5404093; Sharma et al., Immunol Rev. 2017, 280(1):57-73; Lin et al., Nutrients. 2018, 10(1)16; and Maret et al., Int J Mol Sci. 2017, 18(11)).

The G-protein coupled receptor GPR39 was recently discovered as a “Zn sensing receptor” (Hershfinkel et al., Int J Mol Sci. 2018, 19(2)) with putative roles in tissue repair in the gut and skin (Nishida et al., Sci Rep. 2019, 9(1):10842; and Pongkorpsakol et al., Eur J Pharmacol. 2019, 842:306-313). However, while Zn is involved in epithelial cell function in other organs (Shao et al., J Nutr Biochem. 2017, 43:18-26; Emri et al., Metallomics. 2015, 7(3):499-507; and Chasapis et al., Arch Toxicol. 2012, 86(4):521-534), and TECs do express GPR39, this data suggests that its role on TECs regeneration after acute injury is likely indirect through BMP4; although the possibility that GPR39 also mediates effects directly on other stromal cells cannot be excluded. Therefore, one could conclude that Zn is not only needed for thymocyte maturation, but also for thymic repair after acute damage by stimulating the production of BMP4 by ECs.

Thymic regeneration is important following myeloablative conditioning required for successful HCT, after which there is prolonged suppression of T cell immunity. The importance of finding strategies to stimulate thymic-dependent immune reconstitution is highlighted by the correlation between RTEs and clinical outcomes following HCT (Granadier et al., Seminars in Immunopathology. 2021, 43:119-134; and Velardi et al., Nature Reviews Immunology. 2021, 21:277-291). Given its effects on T cell development and on the induction of regenerative factors, it was perhaps not surprising that ZD mice exhibited worse repair following TBI, however, the fact that ZD led to even worse recovery in mice that had fulminant GVHD highlighted its importance for restoration of thymic function after acute damage and highlights the clinical approach for targeting this pathway. Importantly, not only improved thymic function after allogeneic HCT can be shown, but also that this enhanced repair was translated into the circulation with increased numbers of RTEs. However, these findings suggest that the therapeutic benefit of dietary Zn supplementation demands an extended pre-treatment in order for thymocytes to accumulate Zn. Therefore, these findings showing that directly targeting the GPR39 receptor itself with a pharmacological agonist shows this approach is an attractive alternative to induce an equivalent reparative response when given at the time of myeloablative conditioning.

In conclusion, these findings highlight the importance of Zn in steady-state T cell development and reveal a role for Zn in endogenous tissue repair. The studies outlined here not only define important pathways underlying tissue regeneration but also result in innovative clinical approaches to enhance T cell reconstitution in recipients of HCT.

Methods. Mice. 4-6 week-old male or female C57BL16 (CD45.2) or B6.SJL-Ptprca Pepcb/BoyJ (CD45.1) mice were obtained from the Jackson Laboratories (Bar Harbor, USA). RAG2-GFP mice were kindly provided by Dr. Pamela Fink (Monroe et al., Immunity. 1999, 11(2):201-212). Custom-made diets (1 ppm Zinc compared to 35 ppm of control diet) were purchased from Labdiet (St. Louis, Mo.). Zn supplementation was administered orally by dissolving ultrapure Zn sulfate monohydrate (Alfa Aesar, Haverhill, Mass.) in drinking water (1.06 g/ml, which delivered 300 mg/Kg/mouse/day based on average water consumption).

Sublethal TBI was given at a dose of 550cGy with no hematopoietic rescue. HCT mice received 5-10×10⁶ T cell depleted BM cells/recipient. B6 HCT recipients received 1100cGy TBI (2×550cGy); BALB.b recipients received 900cGy (2×450cGy). GVHD was induced with 2×10⁶ T cells/recipient (Bunting et al., Blood. 2017, 129(5):630-642). T cell depletion was performed with CD3 beads (Miltenyi Biotech, Germany. #130-094-973). All TBI experiments were performed with a Cs-137 γ-radiation source.

20 mg TC-G 1008 (Tocris, Bristol, UK.) was given via micropipette-guided administration (Scarborough et al., Brain Behav Immun. 2020, 88:461-470) daily. Dorsomorphin dihydrochloride (12.5 mg/kg/day) was given one day before TBI and then twice daily from day 1. Animals were allowed to acclimatize for at least 2 days before experimentation, which was performed according to Institutional Animal Care and Use Committee guidelines.

Cell Isolation. Individual or pooled single cell suspensions were obtained as previously described (Wertheimer et al., Science Immunology. 2018, 3(19); Dudakov et al., Blood. 2017, 130(7):933-942; and Dudakov et al., Science. 2012, 336(6077):91-95). Cell counts were performed by Z2 particle counter (Beckman Coulter, Pasadena, Calif.), Spark 10M (Tecan, Switzerland) or hemocytometer. CD45-cells were enriched by magnetic bead separation using LS columns and CD45 beads (Miltenyi Biotech, Germany. #130-052-301). Peripheral blood was collected into EDTA capillary pipettes (Drummound Scientific, Broomall, Pa.). Peripheral blood counts were performed on Element Ht5 automatic counter (Heska, Loveland, Colo.).

Cell cultures. exEC were generated as previously described (Seandel et al., Proc Natl Acad Sci USA. 2008, 105(49):19288-19293). Cells were cultured in presence of ultrapure Zn sulfate monohydrate purchased from Alfa Aesar (Haverhill, Mass. #1113809) or sodium pyrythione (Sigma Aldrich, St. Louis, Mo. #H3261-1G). TC-G 1008 was used in cell cultures at a final concentration of 25 μM. Silencing of the zinc receptor Gpr39 was performed by electroporation using Nucleofector electroporation kit (VPI-1001, Lonza; Program M-003, Nucleofector 2b, Lonza), and the GPR39 siRNA Silencer Select (Thermo, Waltham, Mass.). Mouse C9 (cTEC) and TE-71 (mTEC) cells were kindly provided by A. Farr, University of Washington.

Extracellular fractions (“supernatants”) were obtained as previously described (Wertheimer et al., Science Immunology. 2018, 3(19); Dudakov et al., Blood. 2017, 130(7):933-942; and Dudakov et al., Science. 2012, 336(6077):91-95). OP9-DL1 cells were kindly provided by J. C. Zuniga-Pflucker, University of Toronto and cultured as previously described (Holmes et al., Cold Spring Harb Protoc. 2009, 2009(2):pdb prot5156) using lineage-negative BM (using a lineage depletion kit, Miltenyi Biotech, Germany). Flt-3L and IL-7 were purchased from Peprotech (Rocky Hill, N.J.).

ELISA and Western Blot. Cell culture or tissue lysates were prepared in RIPA buffer (Thermo, Waltham, Mass.) as previously described (Wertheimer et al., Science Immunology. 2018, 3(19)) and normalized by BCA assay (Thermo, Waltham, Mass.). BMP4 levels were quantified by ELISA (LSBio, Seattle, Wash.) and read on a Spark 10M plate reader (Tecan, Switzerland). Cortisol levels were measured with ELISA on peripheral blood (R&D systems, Minneapolis, Minn.). Proteins were resolved on 12% SDS-PAGE and transferred onto PVDF membranes (Bio Rad. Hercules, Calif.). Blots were analyzed using the ECL detection system or scanned with an Odyssey Infrared Imager (LI-COR Biosciences, Lincoln, Nebr., USA). In vitro cell proliferation was measured using the CellTiter Non-Radioactive Cell Proliferation Assay (Promega, Madison, Wis.).

Flow cytometry, multidimensional analyses, and FACS sorting. For flow cytometry and cell sorting, surface antibodies against CD45 (30-F11), CD31 (390 or MEC13.3), CD90.2 (30-H12), TER-119 (TER-119), CD4 (RM4-5 or GK1.5), CD8 (53-6.7), TCRβ (H57-597), CD3 (145-2C11), CD44 (IM7), CD25 (PC 61.5), CD62L (MEL14), MHC-II IA/IE (M5/114.15.2), EpCAM (G8.8), Ly51 (6C3), CD11c (HL3), IL-7Ra (A7R34), CCR6 (140706), CD45.1 (A20), CD45.2 (104), ki-67 (16A8), and PDGFRa (APAS) were purchased from BD Biosciences (Franklin Lakes, N.J.), BioLegend (San Diego, Calif.) or eBioscience (San Diego, Calif.). Ulex europaeus agglutinin 1 (UEA-1), conjugated to FITC or Biotin, was purchased from Vector Laboratories (Burlingame, Calif.). GPR39 conjugated to FITC was purchased from Signalway Antibody (College Park, Md.). Red blood cell lysis was performed with ACK buffer (Thermo Scientific, Waltham, Mass.). Flow cytometry was performed on a Fortessa X50 (BD Biosciences, Franklin Lakes, N.J.) and cells were sorted on an Aria II (BD Biosciences) using FACSDiva (BD Biosciences, Franklin Lakes, N.J.). For intracellular cytokine or phosphoprotein analysis, cells were fixed and permeabilized using Fix Buffer I and Phospho-Perm Buffer III from BD Bioscience (Franklin Lakes, N.J.). Fluozin-3 AM was purchased from Thermo Fisher (Waltham, Mass.). Analysis was performed by FlowJo (Treestar Software, Ashland, Oreg.). Gated CD45⁺ T cells were exported in R (version 4.0.2) for further analyses using a custom-made script based on Nowicka et al. ([version 4, peer review: 2 approved]. F1000Research. 2019, 6(748).

PCR and Microarray. Reverse transcription-PCR was performed with iScript Clear gDNA cDNA synthesis kit (Bio Rad, Hercules, Calif.) on CFX96 (Bio Rad, Hercules, Calif.) with iTaq Universal SYBR Green (Bio Rad). Relative amounts of mRNA were calculated by the comparative AC(t) method or as relative expression. SYBR Green gene expression assays for qPCR, including Bmp4 (Mm00432087_m1), Foxn1 (Mm00433948_m1), beta-actin, Gpr39, Top1 were all purchased from Life Technologies (Carlsbad, Calif.) and Bio Rad (Hercules, Calif.). b-actin: synthesized by IDT, F: 5′-CACTGTCGAGTCGCGTCC-3′ (SEQ ID NO: 6), R: 5′-TCATCCATGGCGAACTGGTG-3′ (SEQ ID NO: 7). Top1: synthesized by IDT, sequences provided by PrimerBank, PrimerBank ID 6678399a1. Bmp4: Biorad qMmuCED0046239. Foxn1: Biorad qMmuCED0044924. Microarray data from CD45⁻ cells was analyzed on day 0, 4 and 7 as previously described (Wertheimer et al., Science Immunology. 2018, 3(19)) (GSE106982) and differential gene expression of Gpr39 was calculated.

ICP-MS. Whole thymus tissue was harvested, weighed, and immediately frozen; thymocytes were collected by mechanical dissociation and discarding the remnant stromal component. Each sample was added to trace metal clean plastic vials that had been previously acid leached and rinsed several times with high purity 18 MOhm water. Samples were digested in 1:1 v/v mix of 50% HNO3 and 10% H₂O₂ in a plastic and trace metal clean laminar fume hood. Samples were analyzed on an ICAP RQ ICP-MS (University of Washington, TraceLab) in 2% optima grade nitric acid. Three isotopes of Zn were analyzed and cross referenced for isobaric interferences and Zn-66 was chosen as it exhibited the best signal with the least interference. Precise mass of the initial sample, final sample, and each aliquot was taken into account to calculate concentration of zinc (mg Zn/g thymic tissue or mg Zn/g supernatant). A rhodium internal standard was used to correct for changes in plasma ionization efficiency and external standards including USGS T231 were used to ensure accuracy and traceability.

Statistics. Statistical analysis between two groups was performed with the nonparametric, unpaired Mann-Whitney U test. Statistical comparison between 3 or more groups was performed with the nonparametric, unpaired Kruskall-Wallis test. All statistics were calculated and display graphs were generated in Graphpad Prism. For multiple comparisons, a One-Way ANOVA with Tukey's test was used.

Example 2. 2A. Relationship between Cell Death and Thymic Regeneration after Acute Damage. A major focus of studies has been to identify the pathways mediating endogenous thymic regeneration so that they may be exploited into effective strategies to boost immunity. Innate lymphoid cells (ILCs) and endothelial cells (ECs), through their production of the regeneration factors IL-22 and BM P4, respectively, have profound reparative effects in the thymus after acute injury; and can be utilized individually as therapeutic strategies of immune regeneration (Dudakov et al., Science 336, 91-95, 2012; Wertheimer et al., Science Immunology 3, 2018; and Dudakov et al., Blood 130, 933-942, 2017). However, both of these pathways ultimately target thymic epithelial cells (TECs), the stromal mediator of T cell development (Anderson et al., Nat Rev Immunol 1, 31-40, 2001; Takahama, Nat Rev Immunol 6, 127-135, 2006; and Petrie and Zuniga-Pflucker, Annu Rev Immunol 25, 649-679, 2007), and the expression of Foxn1. FOXN1 is the quintessential thymic transcription factor as it is not only crucial for functionally enabling TECs to support T cell development (Vaidya et al., Eur J Immunol 46, 1826-1837, 2016; Nehls et al., Science 272, 886-889, 1996; and Swann et al., Cell Reports 8, 1184-1197, 2014), but for ongoing TEC maintenance; and its declining expression likely contributes to age-related thymic involution (Corbeaux et al., Proceedings of the National Academy of Sciences 107, 16613-16618, 2010; Chen et al., Blood 113, 567-574, 2009; Nowell et al., PLoS Genet 7, e1002348, 2011; Cheng et al., The Journal of biological chemistry 285, 5836-5847, 2010; and Rode et al., J Immunol 195(12)5678-87, 2015). Furthermore, there is increasing evidence that FOXN1 is also important during thymic regeneration (Zook et al., Blood 118(22), 5723-5731, 2011; Bredenkamp et al., Development 141, 1627-1637, 2014; and Song et al., European Journal of Immunology 46:1518-1528, 2016). Given its central role in thymic function, from development through to regeneration, FOXN1 is an attractive target to specifically mediate thymic regeneration. This example is directed to understanding mechanisms promoting FOXN1 induction after damage, and thus driving TEC-mediated thymic regeneration to develop therapeutic strategies to improve thymus function after both acute and chronic damage such as that caused during the conditioning for hematopoietic cell transplant (HCT).

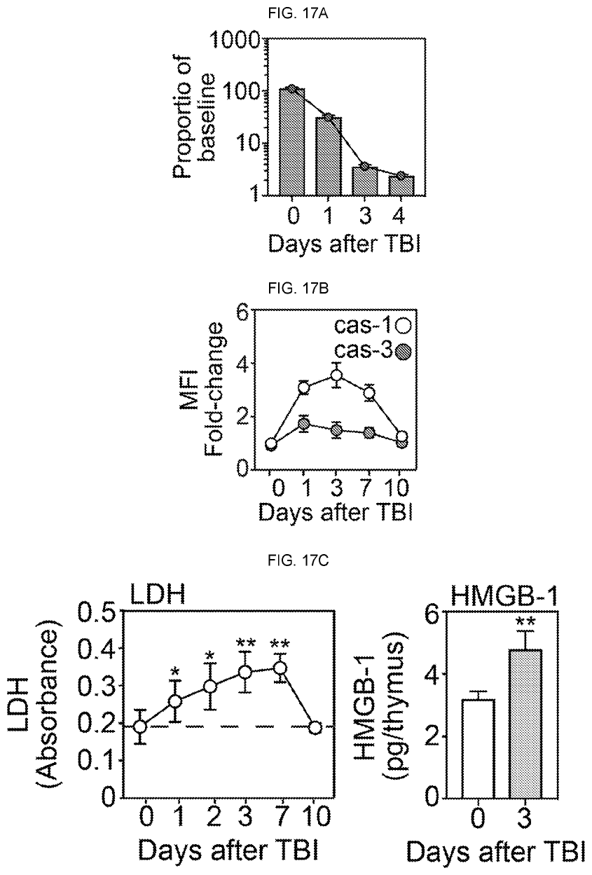

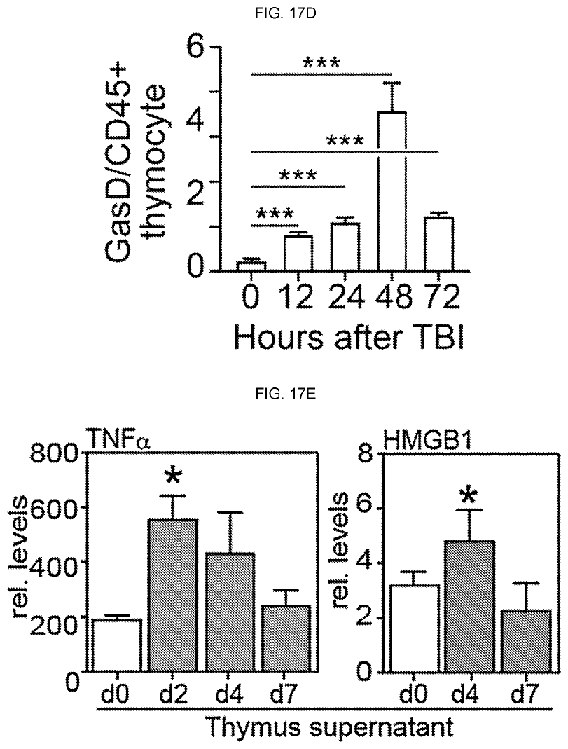

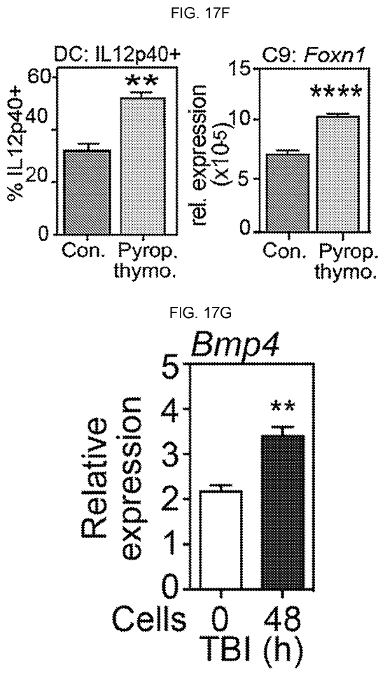

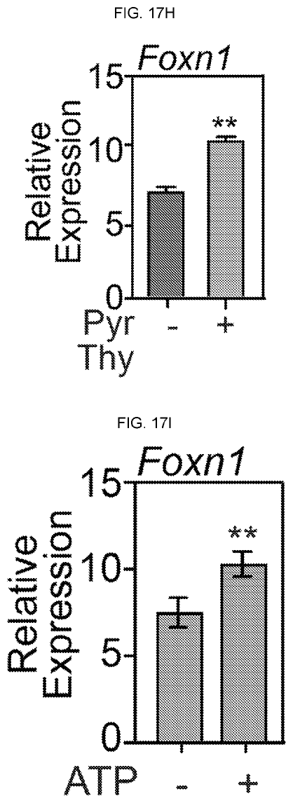

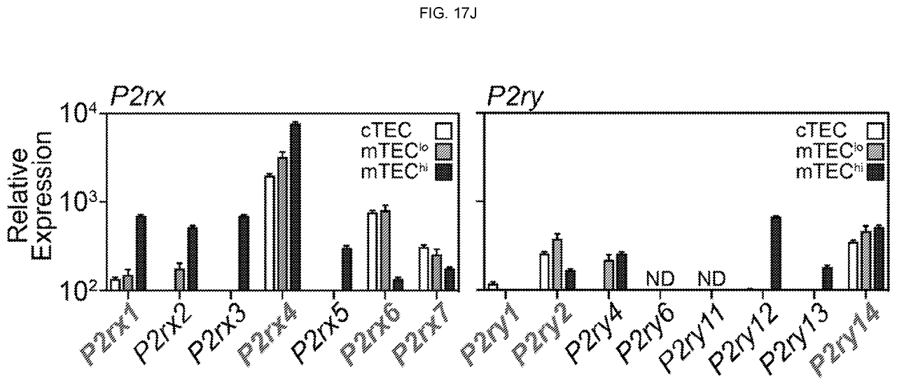

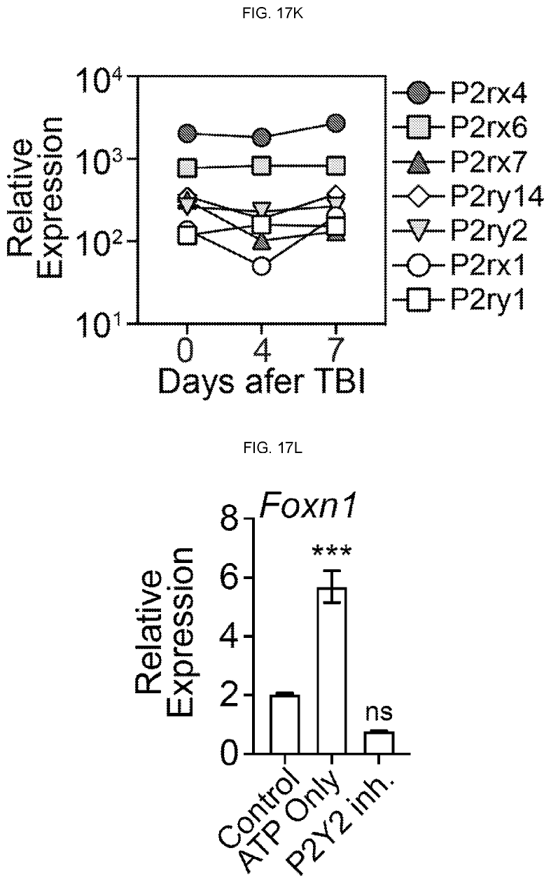

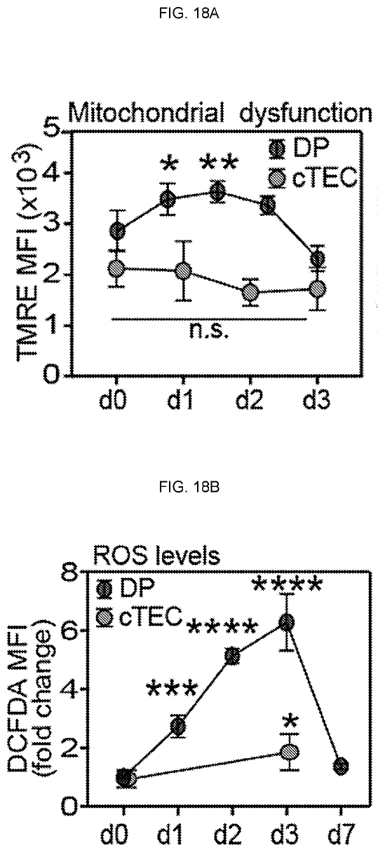

Although DP thymocytes are the most numerous cell population in the thymus (80-85% of the thymus), in steady-state thymopoiesis most DP thymocytes undergo immunogenically silent apoptosis as a results of positive selection. Apoptotic thymocytes can suppress the production of regenerative factors, such as Bmp4 and IL-23, in ECs and DCs respectively (FIG. 16A). The presence of phosphatidylserine (PS) on the surface of apoptotic thymocytes was identified to mediate this suppressive effect; with PS availability decreasing dramatically in the thymus after damage (FIG. 16B), commensurate to the depletion of (apoptotic) thymocytes. Functionally, inhibition of the interaction between PS and either ECs or DCs by targeting TAM receptors, cell surface receptors that detect PS48, resulted in the reversal of suppression of apoptotic thymocytes and the increased production of Bmp4 and IL-23 (FIG. 16C). While there is typically abundant apoptosis in the steady-state thymus during positive selection, there is considerable evidence that acute insults like radiation and chemotherapy can lead to immunogenic cell death (ICD) such as pyroptosis or necroptosis; lytic forms of cell death that lead to the release of intracellular contents including damage-associated molecular patterns (DAMPs) that can elicit an immune response (Galluzzi et al., Cell Death Differ 25, 486-541, 2018; Simader et al., Cell death & disease 10, 729-729, 2019; and Kroemer et al., Annu Rev Immunol 31, 51-72, 2013). Consistent with this, a distinct induction of pyroptotic cell death in thymocytes after damage was found, as demonstrated by the detection of increased caspase 1 cleavage (pyroptotic caspase) in comparison to caspase 3 cleavage (apoptotic caspase) (FIG. 17A), accompanied by increased levels of the classical pyroptosis marker lactate dehydrogenase (LDH, FIG. 17B). Furthermore, increased extracellular levels of high mobility group box 1 (HMGB-1) and tumor necrosis factor α (TNFα) [canonical damage-associated molecular patterns (DAMPs) were released during ICD49] early after acute damage caused by TBI (FIG. 17E). However, intriguingly, pyroptotic thymocytes can directly induce IL-23 in DCs, and Foxn1 in C9 cells (a cTEC cell line) (FIG. 17F), further strengthening that cell-cell communication drives thymic regeneration after damage via induction of regenerative factors as well as directly directing TEC function. Furthermore, these findings also shiow that this communication is mediated by secreted factors from pyroptotic cells. Therefore, it was hypothesized that while under steady-state conditions, when thymocytes are typically undergoing a generally immune silent apoptotic cell death (Hernandez et al., Curr Opin Cell Biol 22, 865-871, 2010; and Galluzzi et al., Cell Death Differ 25, 486-541, 2018), there is no elicitation of the regenerative response; but after acute insult, the release of intracellular contents during ICD actively induces the regenerative response. In fact, there is evidence in other tissues that this is the case as DAMPs have been implicated in the induction of a regeneration in skin, liver, kidney, muscle, and heart (Simader et al., Cell death & disease 10, 729-729, 2019; Venereau et al., Front Immunol. 6:422, 2015; Anders and Schaefer, Journal of the American Society of Nephrology: JASN 25, 1387-1400, 2014; Yang and Tonnesseen, Hepatol Int 13, 42-50, 2019; and Wilgus, Curr Pathobiol Rep 6, 55-60, 2018).

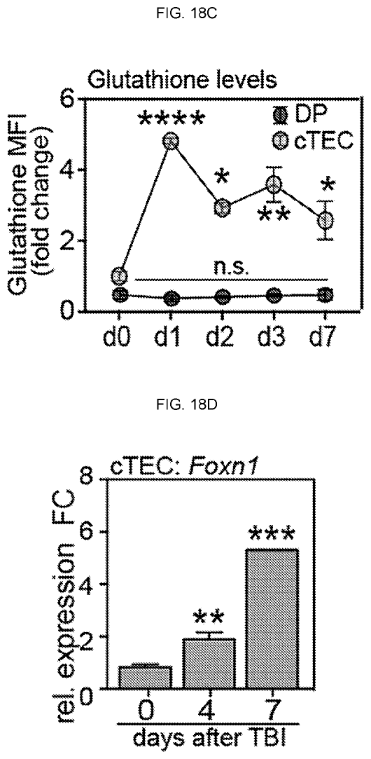

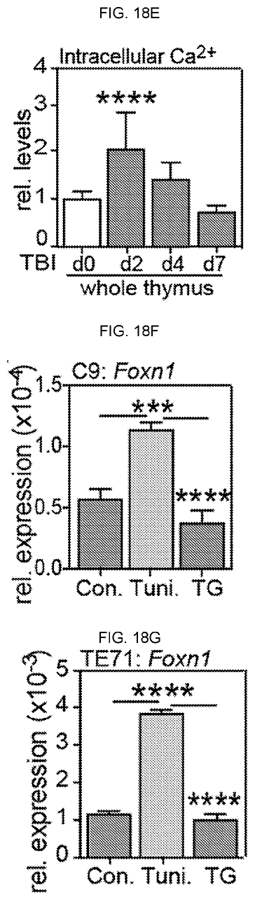

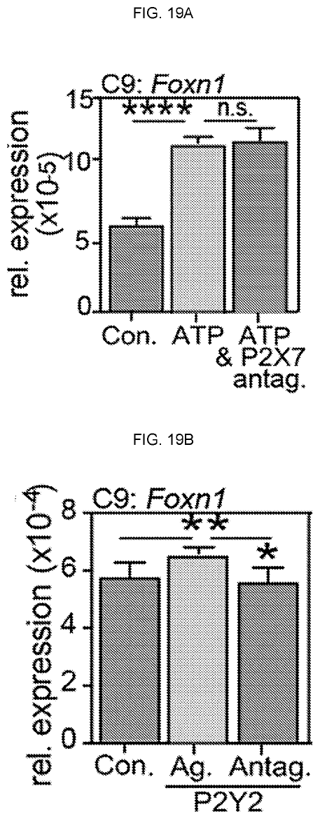

Given the preferential induction of pyroptotic cell death in DP thymocytes after damage (FIG. 17A), the findings that stromal cells are more radio-resistant than DP thymocytes (Wertheimer et al., Sci Immunol 3(19), 2018), and evidence for mitochondrial-induced pyroptosis (Wang et al., J Mol Cell Biol 11, 1069-1082, 2019), an understanding of the differential metabolic responses to acute injury in DPs and cTECs (the supporting stromal cell for DP thymocytes and the TEC most involved with driving regeneration) was sought. The data demonstrated hyperpolarization of the mitochondrial membrane potential, increased ROS and lower levels of the antioxidant glutathione in DPs, compared with cTECs (FIGS. 18A-18C), indicating damage-induced metabolic regulation of thymocyte death after damage. Intracellular Ca2+ is a critical regulator of mitochondrial metabolism (Denton and McCormack, Biochem Soc Trans 8, 266-268, 1980) and has also been identified to play a central role in tissue regeneration (Aihara et al., J Biol Chem 288, 33585-33597, 2013; and Taira et al., J Exp Pharmacol 8, 21-33, 2016). Interestingly Ca2+ has also been found to be involved in the membrane repair response to inhibit pyroptosis (Ruhl et al., Science (New York, N.Y.) 362, 956-960, 2018). Consistent with a role for Ca2+ signaling contributing toward TEC regeneration, increased Ca2+ levels were identified in the thymus that precedes FOXN1 induction in cTECs (FIGS. 18D and 18E) after damage. Furthermore, consistent with these findings, stimulating the intracellular release of Ca2+, using tunicamycin, induced Foxn1 expression in cTEC (C9) mTEC (TE71) cell lines (FIGS. 18F and 18G). This was reversed upon inhibition of release of ER Ca2+ with thapsigargan (FIGS. 18F and 18G). Therefore, damage-induced metabolic dysregulation facilitates pyroptotic cell death in thymocytes, and intracellular Ca2+ levels regulate metabolic stability and survival of TECs.

2B. The effects of DAMPs on TEC function and thymic regeneration. Damage-induced ICD promotes Foxn1 expression in cTECs (C9s) in vitro. One mechanism by which this may occur is that after acute injury the release of intracellular contents, such as DAMPs, during ICD actively mediates TEC survival via the induction of FOXN1. In fact, as stated previously, there is evidence in other tissues that this is the case as DAMPs have been implicated in the induction of a regenerative response in skin, liver, kidney, muscle, and heart (Simader et al., Cell death & disease 10, 729-729, 2019; Venereau et al., Front Immunol. 6:422, 2015; Anders and Schaefer, Journal of the American Society of Nephrology: JASN 25, 1387-1400, 2014; Yang and Tonnesseen, Hepatol Int 13, 42-50, 2019; and Wilgus, Curr Pathobiol Rep 6, 55-60, 2018). A classic DAMP that is released during ICD is ATP49 (Venereau et al., Frontiers in Immunology 6, 2015), which can activate Ca2+ signalling through cell surface purinergic receptors (May et al., Biochemical pharmacology 71, 1497-1509, 2006). Two subsets of purinergic receptors have been described; ligand-gated ionotropic P2X receptors, which induce Ca2+ influx; and metabotropic G-coupled P2Y receptors, which induce Ca2+ efflux from the endoplasmic reticulum (ER) (Dubyak and el-Moatassim, Am J Physiol 265, C577-606, 1993; and Abbracchio et al., Pharmacol Rev 58, 281-341, 2006). Coupled with data demonstrating the central role of Ca2+ in FOXN1 induction, DAMP-induced Ca2+ influx or efflux in TECs facilitates thymus regeneration.

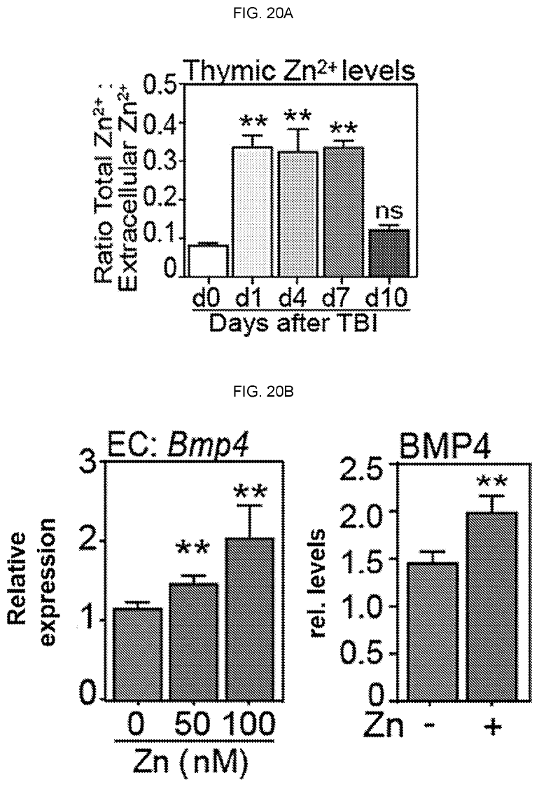

Purinergic receptor expression is heterogeneous between thymic cell subsets, with P2Y2 expressed among all subsets of TECs, with a less homogeneous distribution of P2X7 (Bisaggio et al., Cell Mol Biol (Noisy-le-grand) 47:19-31, 2001). Interestingly, ATP is a potent inducer of FOXN1 in cTECs (C9s), however, this effect was not mediated via P2X7 (FIG. 19A). Activation of P2Y2 with a P2Y2 agonist (MRS 2768) leads to an increase in Foxn1 expression which is reversed upon inhibition with a P2Y2 antagonist (AR-C 1182925XX) (FIG. 19B). Therefore, it is disclosed that activating P2Y2 enhances FOXN1-driven thymus regeneration after damage.

To further confirm this disclosure, TECs will first be freshly isolated and incubated in the presence of ATP or the P2Y2 agonist. Freshly isolated TECs rapidly downregulate FOXN1 and are difficult to culture (Wertheimer et al., Sci Immunol 3(19), 2018); therefore, Foxn1 expression will be assessed as well as viability and survival in these cultures (as previously described Dudakov et al. and Wertheimer et al.). To determine the specificity of P2Y2 activation in the regenerative response in vivo, mice deficient in P2Y2R (P2Y2R−/−) (obtained from Jackson Laboratories) will be used and thymic recovery at days 0, 4, 7, 14, & 28 after sub-lethal total body irradiation (SL-TBI) will be assessed. Additionally, cTECs & mTECs from WT and P2Y2R−/− mice at days 0, 1, 2, 3 & 7 after SL-TBI will be purified and Foxn1 and D114 expression by qPCR will be quantified. Furthermore, to demonstrate Ca2+-dependent P2Y2 regenerative mechanisms Ca2+ assays will be carried out using the fluorescent intracellular Ca2+ dye Fluo-3. Specifically, cTECs and mTECs from untreated WT and P2Y2R−/− mice will be purified and co-cultured overnight with combinations of pyroptotic thymocytes, ATP, P2Y2 agonists, and P2Y2 antagonists, as above. Survival, proliferation, expression of Foxn1 and D114, and intracellular Ca2+ levels will be determined and WT TECs will respond to the presence of pyroptotic cells or DAMPs by inducing FOXN1, and there will be attenuated induction of FOXN1 in TECs deficient for P2Y2R.

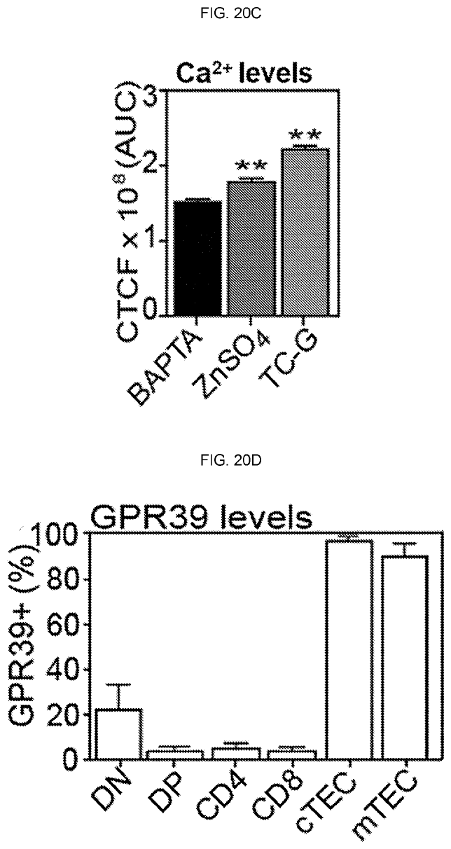

Of note, the release of Zn2+ from dying thymocytes can act as a DAMP to induce the expression of the regenerative factor BM P4 by ECs (FIGS. 20A and 20B). Furthermore, agonism of the Zn2+ receptor GPR39 incudes Ca2+ influx (Sunuwar et al., Philos Trans R Soc Lond B Biol Sci 371, 2016) (FIG. 20C). Given the extensive expression GPR39 on TECs (FIG. 20D), direct activation of GPR39, with the specific agonist TC-G 1008, presents an attractive therapeutic target that mediates regeneration as a monotherapy or in combination with P2Y2 agonism.

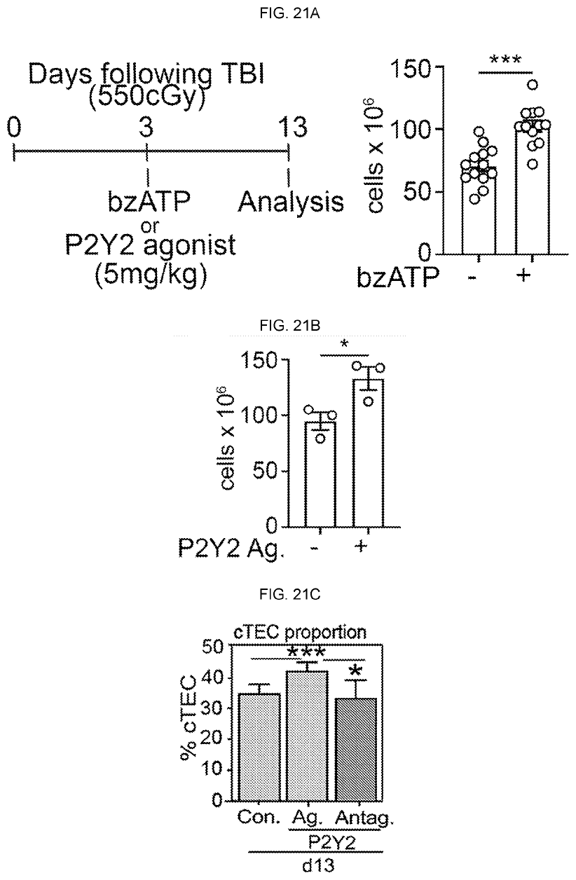

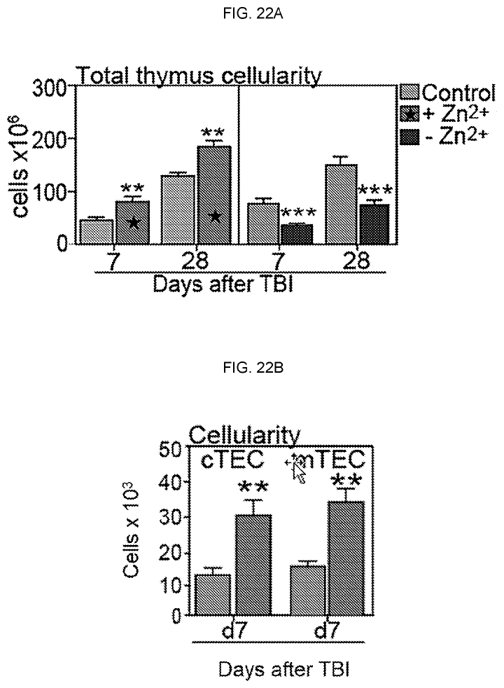

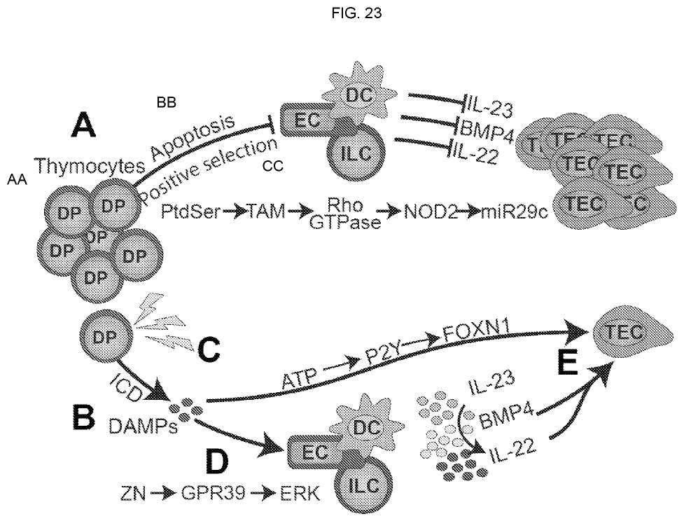

2C: Administration of either the P2Y2 agonist MRS 2768 or ATP in vivo, by intraperitoneal injection at d3 following SL-TBI, resulted in superior thymic regeneration (FIGS. 21A and 21B), and this enhancement of thymic recovery is reflected in increased cTEC numbers following P2Y2 agonist (FIG. 21C). Therefore, targeting P2Y2 offers an innovative therapeutic target to enhance thymic regeneration by directly targeting FOXN1 in TECs.

To further confirm that activating P2Y2 signaling improves thymic function and T cell reconstitution after clinically relevant models of allogeneic HCT (alto-HCT), MRS 2768 and the P2Y2 antagonist AR-C 1182925XX will be administered, to young (2 mo), middle-aged (9 mo), and old (24 mo) WT recipients using two models of allo-HCT: an MHC mismatched model (T cell depleted B10.BR BM transplanted into B6 recipients); as well as a MHC-matched, minor antigen mismatched (LP/J BM into B6 recipients) as previously published (Dudakov et al., Science 336, 91-95, 2012; Dudakov et al., Blood 130, 933-942, 2017; Fischer et al., Science Translational Medicine 9, 2017; Jenq et al., Biol Blood Marrow Transplant 21, 1373-1383, 2015; Shono et al., Science Translational Medicine 8, 339ra371-339ra371, 2016; Lindemans et al., Nature 528, 560-564, 2015; Velardi et al., The Journal of Experimental Medicine 211, 2341-2349, 2014; Hartrampf et al., Blood 121, 1906-1910, 2013; Hanash et al., Immunity 37, 339-350, 2012; Jenq et al., J Exp Med 209, 903-911, 2012; and Hanash et al., Blood 118, 446-455, 2011). Thymic function will be assessed at days 4, 7, 10, 14, 28, and 42 by: (1) enumerating hematopoietic and stromal cells by flow cytometry and the composition of thymus subsets will be enumerated (including stromal cells such as ECs, DCs, TECs, and fibroblasts, as well as thymocyte subpopulations like DP thymocytes, CD4+, and CD8⁺ T cells); (2) measuring intrathymic levels of BMP4, IL-23, and IL-22, by ELISA; and (3) isolating TECs and assessing their expression of key thymopoietic factors such as D114, Kitl, 117, Cxc112, and Cc125. P2Y2 agonism will enhance early recovery of thymic cellularity and upregulate production of thymopoietic growth factors and will assess thymus cellularity and the expression of Foxn1 and D114.

To comprehensively assess peripheral T cell reconstitution, in vitro and in vivo assays will be performed to assess (1) thymic export to assess numbers of recent thymic emigrants using donors expressing the green fluorescent protein (GFP) under the control of RAG2 (Rag2pGFP) (Wertheimer et al., Science Immunology 3, 2018) in HCT, by analysis of peripheral blood, lymph nodes, & spleen at days 28 and 42; (2) peripheral T cell phenotype by enumerating peripheral T cells assessing donor/host contribution and naive/memory/Treg phenotype at days 0, 28, and 42; timepoints which cover the emergence and establishment of T cells from the thymus to the periphery; and (3) functional capacity to respond to stimulation by measuring proliferation and cytokine production of splenic or lymph node T cells stimulated with αCD3/αCD28 or PMA/lonomycin. To determine T cell function in an antigen-driven model in vivo, transplanted mice will be challenged with 2×105 plaque forming units (PFU) lymphocytic choriomeningitis virus (LCMV)-Armstrong 21 days after HCT and perform PFU assays on spleen at d28 and d42. P2Y2 agonism, and its impacts on thymic regeneration, will lead to enhanced T cell function after allo-HCT. Given that this therapy will lead to the direct activation FOXN1-mediated thymic recovery, as a comparison administration of recombinant IL-22 or exECs will be performed using protocols that have previously been demonstrated as effective for thymic reconstitution (Dudakov et al., Science 336, 91-95, 2012; Wertheimer et al., Science Immunology 3, 2018; and Dudakov et al., Blood 130, 933-942, 2017).

Summary of Example 2. Recent studies have identified two key pathways driving thymic regeneration; centered on the secretion of BMP4 by endothelial cells (ECs) and IL-22 by innate lymphoid cells (Dudakov 2012 Science 336:91; Dudakov 2017 Blood 130:933; Wertheimer 2018 Sci Immunol 3:19). However, the specific regulatory mechanisms that trigger these regeneration-associated factors after damage are still not completely understood. Previous work identified that the presence of homeostatic apoptotic CD4+CD8+(DP) thymocytes, as apoptotic thymocytes form the bulk of developing T cells, suppress the production of IL-23 in dendritic cells (DCs), a key downstream mediator for IL-22, and BMP4 in ECs, and that the depletion of apoptotic thymocytes after damage precedes the production of these regenerative factors. Therefore, together with findings that the metabolic needs of key thymus populations alter drastically following injury due to damage-induced metabolic remodeling, it was hypothesized that further to the loss of DP-specific suppression, metabolic dysfunction in DPs after damage triggers mitochondrial-induced pyroptotic cell death, which can directly promote regeneration of the thymus.

Consistent with this scenario, the data shows increased levels of cl-caspase 1 (pyroptotic caspase) and a decrease in cl-caspase 3 (apoptotic caspase) in DPs after SL-TBI (550 cGy), demonstrating a preferential induction of pyroptotic cell death in DPs after damage. Furthermore, there wea an increase in extracellular lactate dehydrogenase (LDH) levels, HMGB-1 and TNFα [canonical damage-associated molecular patterns (DAMPs) released during ICD] acutely after damage caused by SL-TBI. Given previous findings that stromal cells are more radio-resistant than DP thymocytes (Wertheimer 2018 Sci Immunol 3:19), and evidence for mitochondrial-induced pyroptosis, hyperpolarization of the mitochondrial membrane potential accompanied by increased levels of reactive oxygen species (ROS) in DPs was identified, an effect not observed in TECs, suggesting metabolic stability confers protection against acute damage. Furthermore, co-culture of pyroptotic thymocytes results in increased IL12p40+ DCs and increased Foxn1 expression in TECs, strengthening the conclusion that cell-cell communication drives thymic regeneration after damage by inducing regenerative factors as well as directly promoting TEC function via secreted factors from pyroptotic DPs. One way in which DAMPs, such as ATP, can initiate cell signaling is by the activation of cell surface purinergic receptors, including P2Y2 which is widely expressed on TECs. The current disclosure provides that in vitro treatment with P2Y2 agonist increases Foxn1 in cTECs, and antagonism reverses this effect. As P2Y2 activation promotes Ca2+ efflux from the ER, the current disclosure further demonstrates that stimulating the intracellular release of Ca2+, using tunicamycin, induced Foxn1 expression in cTECs, which was reversed upon inhibition of Ca2+ release. Importantly, the current disclosure also demonstrates that this pathway can be therapeutically targeted by activating P2Y2 signaling in vivo with MRS 2768 or ATP, thus enhancing thymus cellularity and expanding cTECs in models of acute injury.

These findings not only reveal a novel metabolic-mediated molecular mechanism governing tissue regeneration; but by targeting FOXN1 directly, also offers a potentially superior therapeutic strategy for boosting thymic regeneration and T cell reconstitution after damage such as that caused by HCT, infection or cytoreductive therapy.

In particular embodiments, an administered compound that activates a receptor results in physiological effects that occur when the receptor is bound by its natural endogenous activating ligand(s).

Variants of protein, nucleic acid, and gene sequences disclosed herein include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, N Y (1988); Biocomputing: Informatics and Genome Projects (Smith, D. W., ed.) Academic Press, N Y (1994); Computer Analysis of Sequence Data, Part I (Griffin, A. M., and Griffin, H. G., eds.) Humana Press, N J (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wis.). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wis.); BLASTP, BLASTN, BLASTX (Altschul, et al., J. Mol. Biol. 215:403-410 (1990); DNASTAR (DNASTAR, Inc., Madison, Wis.); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

Each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the ability to obtain a claimed effect according to a relevant experimental method described in the current disclosure.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method of treating a subject in need of thymic regeneration comprising administering a therapeutically effective amount of TC-G 1008 to the subject, thereby treating the subject in need of thymic regeneration.

2. The method of claim 1, wherein the subject is in need of thymic regeneration due to age or infection.

3. The method of claim 1, wherein the subject is in need of thymic regeneration due to an immune-compromised status due to a treatment.

4. The method of claim 3, wherein the treatment is a cancer treatment.

5. The method of claim 1, further comprising administering a therapeutically effective amount of MRS2768.

6. A method of treating a subject in need of promoted thymic function comprising administering a therapeutically effective amount of a composition comprising a GPR39 receptor-activating compound and/or a purinergic receptor-activating compound thereby treating the subject in need thereof.

7. The method of claim 6, wherein the treating promotes thymic regeneration.

8. The method of claim 6, wherein the subject is in need of promoted thymic function based on age or an immune-compromised status due to a treatment.

9. The method of claim 6, wherein the subject is in need of promoted thymic function based on infection or a cancer treatment.

10. The method of claim 6, wherein the GPR39 receptor-activating compound comprises TC-G 1008, LY2784544, GSK2636771, obestatin, AZ1395, AZ4237, AZ7914, AZ4502, AZ9309, AZ2097, Compound 1, and/or Compound 15.

11. The method of claim 6, wherein the purinergic receptor-activating compound activates a P2Y2 purinergic receptor, a P2Y1 purinergic receptor, a P2Y14 purinergic receptor, a P2Y6 purinergic receptor, a P2X7 purinergic receptor, a P2X3 purinergic receptor, a P2X4 purinergic receptor, a P2X1 purinergic receptor, a P2X2 purinergic receptor, a P2X5 purinergic receptor, a P2X6 purinergic receptor, a P2Y4 purinergic receptor, a P2Y11 purinergic receptor, a P2Y12 purinergic receptor, or a P2Y13 purinergic receptor.

12. The method of claim 6, wherein the purinergic receptor-activating compound activates the P2Y2 purinergic receptor.

13. The method of claim 12, wherein the purinergic receptor-activating compound that activates the P2Y2 purinergic receptor comprises ATP, MRS 2768, MRS 2698, Denufosol, Diquafosol, 4-thio-UTP, 5BrUTP, Ap4A, uridine triphosphate (UTP), UTPγS, 2-thioUTP, and/or PSB1114.

14. The method of claim 6, wherein the purinergic receptor-activating compound activates the P2Y1 purinergic receptor.

15. The method of claim 14, wherein the purinergic receptor-activating compound that activates the P2Y1 purinergic receptor comprises MRS2170, MRS2267, MRS2279, [³H]2MeSADP, MRS2365, 2-CI-ADP(α-BH3), compound 3a, ADPβS, Ap3a, Ap5a, 2′,3′-ddATP, dATPαS, ATPγS, 2MeSATP, ATP, ADP, and/or [35S]ADPβS.

16. The method of claim 6, wherein the purinergic receptor-activating compound activates the P2Y14 purinergic receptor.

17. The method of claim 16, wherein the purinergic receptor-activating compound that activates the P2Y14 purinergic receptor comprises a uridine diphosphate (UDP), a UDP-sugar, an α.β-methylene-2-thio-UDP, an MRS4183, an MRS2905, a 2-thio-UDP, an MRS2802, and/or an MRS2690.

18. The method of claim 17, wherein the UDP-sugar comprises UDP-glucose, UDP-galactose, UDP-glucoronic acid, and/or UDP-N-acetylglucosamine.

19. The method of claim 6, further comprising administering a therapeutically effective amount of a composition comprising an inhibitor selected from a NOD2 inhibitor, a Rho GTPase inhibitor, and/or an miR29c inhibitor.

20. The method of claim 19, wherein the NOD2 inhibitor comprises ponatinib, regorafenib, gefitinib, curcumin, a sesquiterpene lactone, a pseudopterosin, a polyunsaturated fatty acid, a benzimidazole diamide, and/or a hydrophenalene-chromium complex.

21. The method of claim 20, wherein the sesquiterpene lactone comprises parthenolide and/or helenalin.

22. The method of claim 20, wherein the pseudopterosin comprises pseudopterosin A.

23. The method of claim 20, wherein the polyunsaturated fatty acid comprises docosahexaenoic acid (DHA) and/or eicosapentaenoic acid (EPA).

24. The method of claim 20, wherein the benzimidazole diamide comprises GSK669 and/or GSK717.

25. The method of claim 19, wherein the Rho GTPase inhibitor comprises isoflavones, (E)-3-(3-(ethyl(quinolin-2-yl)amino)phenyl)acrylic acid, (E)-3-(3-(butyl(quinolin-2-yl)amino)phenyl)acrylic acid, C3 transferase, ZCL 278, Rhosin hydrochloride, ML 141, CASIN, p120 catenin, MLS000532223, and/or MLS000573151.

26. The method of claim 19, wherein the Rho GTPase inhibitor comprises a RhoA inhibitor and/or a Rac1 inhibitor.

27. The method of claim 26, wherein the Rac1 inhibitor comprises EHT 1864, Rac1 Inhibitor W56, NSC 23766, EHop 016, 6-mercaptopurine (6-MP), and/or 6-thioguanosine-5′-triphosphate (6-T-GTP).

28. The method of claim 19, wherein the miR29c inhibitor comprises a complementary interfering RNA sequence.

29. The method of claim 19, wherein the miR29c inhibitor comprises SEQ ID NO: 5.

30. The method of claim 19, wherein the miR29c inhibitor comprises a PPAR-γ agonist.

31. The method of claim 25, wherein the PPAR-γ agonist comprises pioglitazone; 15-deoxy-delta-12,14-PGJ₂; and/or thiazolidinedione.

32. A method of upregulating FOXN1, IL-22, IL-23, and/or BMP4 in a subject in need thereof comprising administering a therapeutically effective amount of a composition comprising a GPR39 receptor-activating compound and/or a purinergic receptor-activating compound to the subject thereby upregulating FOXN1, IL-22, IL-23, and/or BMP4 in the subject.

33. The method of claim 32, wherein the subject is in need of upregulating FOXN1, IL-22, IL-23, and/or BMP4 to promote thymic function.

34. The method of claim 32, wherein the subject is in need of upregulating FOXN1, IL-22, IL-23, and/or BMP4 to promote thymic regeneration.

35. The method of claim 33, wherein the subject is in need of promoted thymic function based on age or an immune-compromised status due to a treatment.

36. The method of claim 33, wherein the subject is in need of promoted thymic function based on infection or a cancer treatment.

37. The method of claim 32, wherein the GPR39 receptor-activating compound comprises TC-G 1008, LY2784544, GSK2636771, obestatin, AZ1395, AZ4237, AZ7914, AZ4502, AZ9309, AZ2097, Compound 1, and/or Compound 15.

38. The method of claim 32, wherein the purinergic receptor-activating compound activates a P2Y2 purinergic receptor, a P2Y1 purinergic receptor, a P2Y14 purinergic receptor, a P2Y6 purinergic receptor, a P2X7 purinergic receptor, a P2X3 purinergic receptor, a P2X4 purinergic receptor, a P2X1 purinergic receptor, a P2X2 purinergic receptor, a P2X5 purinergic receptor, a P2X6 purinergic receptor, a P2Y4 purinergic receptor, a P2Y11 purinergic receptor, a P2Y12 purinergic receptor, and/or a P2Y13 purinergic receptor.

39. The method of claim 32, wherein the purinergic receptor-activating compound activates the P2Y2 purinergic receptor.

40. The method of claim 39, wherein the purinergic receptor-activating compound that activates the P2Y2 purinergic receptor comprises ATP, MRS 2768, MRS 2698, Denufosol, Diquafosol, 4-thio-UTP, 5BrUTP, Ap4A, uridine triphosphate (UTP), UTPγS, 2-thioUTP, and/or PSB1114.

41. The method of claim 32, wherein the purinergic receptor-activating compound activates the P2Y1 purinergic receptor.

42. The method of claim 41, wherein the purinergic receptor-activating compound activates that the P2Y1 purinergic receptor comprises MRS2170, MRS2267, MRS2279, [3H]2MeSADP, MRS2365, 2-CI-ADP(α-BH3), compound 3a, ADPβS, Ap3a, Ap5a, 2′,3′-ddATP, dATPαS, ATPγS, 2MeSATP, ATP, ADP, and/or [35S]ADPβS.

43. The method of claim 32, wherein the purinergic receptor-activating compound activates the P2Y14 purinergic receptor.

44. The method of claim 43, wherein the purinergic receptor-activating compound that activates the P2Y14 purinergic receptor comprises a uridine diphosphate (UDP), a UDP-sugar, an α.β-methylene-2-thio-UDP, an MRS4183, an MRS2905, a 2-thio-UDP, an MRS2802, and/or an MRS2690.

45. The method of claim 44, wherein the UDP-sugar comprises UDP-glucose, UDP-galactose, UDP-glucoronic acid, and/or UDP-N-acetylglucosamine.

46. The method of claim 32, further comprising administering a therapeutically effective amount of a composition comprising an inhibitor comprising a NOD2 inhibitor, a Rho GTPase inhibitor, and/or an miR29c inhibitor.

47. The method of any of claim 46, wherein the NOD2 inhibitor comprises ponatinib, regorafenib, gefitinib, curcumin, a sesquiterpene lactone, a pseudopterosin, a polyunsaturated fatty acid, a benzimidazole diamide, and/or a hydrophenalene-chromium complex.

48. The method of claim 47, wherein the sesquiterpene lactone comprises parthenolide and/or helenalin.

49. The method of claim 47, wherein the pseudopterosin comprises pseudopterosin A.

50. The method of claim 47, wherein the polyunsaturated fatty acid comprises docosahexaenoic acid (DHA) and/or eicosapentaenoic acid (EPA).

51. The method of claim 47, wherein the benzimidazole diamide comprises GSK669 and/or GSK717.

52. The method of claim 46, wherein the Rho GTPase inhibitor comprises isoflavones, (E)-3-(3-(ethyl(quinolin-2-yl)amino)phenyl)acrylic acid, (E)-3-(3-(butyl(quinolin-2-yl)amino)phenyl)acrylic acid, C3 transferase, ZCL 278, Rhosin hydrochloride, ML 141, CASIN, p120 catenin, MLS000532223, and/or MLS000573151.

53. The method of claim 46, wherein the Rho GTPase inhibitor comprises a RhoA inhibitor and/or a Rac1 inhibitor.

54. The method of claim 53, wherein the Rac1 inhibitor comprises EHT 1864, Rac1 Inhibitor W56, NSC 23766, EHop 016, 6-mercaptopurine (6-MP), and/or 6-thioguanosine-5′-triphosphate (6-T-GTP).

55. The method of claim 46, wherein the miR29c inhibitor comprises a complementary interfering RNA sequence.

56. The method of claim 46, wherein the miR29c inhibitor comprises SEQ ID NO: 5.

57. The method of claim 46, wherein the miR29c inhibitor comprises a PPAR-γ agonist.

58. The method of claim 57, wherein the PPAR-γ agonist comprises pioglitazone; 15-deoxy-delta-12,14-PGJ₂; and/or thiazolidinedione.

59. The method of claim 32, wherein the upregulating promotes thymic function in the subject.

60. The method of claim 32, wherein the upregulating promotes thymic regeneration in the subject.

61. A composition comprising a therapeutically effective amount of a GPR39 receptor-activating compound and/or a purinergic receptor-activating compound wherein the therapeutically effective amount(s) promotes thymic function.

62. The composition of claim 61, wherein the therapeutically effective amount(s) that promotes thymic function promotes thymic regeneration.

63. The composition of claim 61, wherein the GPR39 receptor-activating compound comprises TC-G 1008, LY2784544, GSK2636771, obestatin, AZ1395, AZ4237, AZ7914, AZ4502, AZ9309, AZ2097, Compound 1, and/or Compound 15.

64. The composition of claim 61, wherein the purinergic receptor-activating compound activates a P2Y2 purinergic receptor, a P2Y1 purinergic receptor, a P2Y14 purinergic receptor, a P2Y6 purinergic receptor, a P2X7 purinergic receptor, a P2X3 purinergic receptor, a P2X4 purinergic receptor, a P2X1 purinergic receptor, a P2X2 purinergic receptor, a P2X5 purinergic receptor, a P2X6 purinergic receptor, a P2Y4 purinergic receptor, a P2Y11 purinergic receptor, a P2Y12 purinergic receptor, or a P2Y13 purinergic receptor.

65. The composition of claim 61, wherein the purinergic receptor-activating compound activates the P2Y2 purinergic receptor.

66. The composition claim 65, wherein the purinergic receptor-activating compound that activates the P2Y2 purinergic receptor comprises ATP, MRS 2768, MRS 2698, Denufosol, Diquafosol, 4-thio-UTP, 5BrUTP, Ap4A, uridine triphosphate (UTP), UTPγS, 2-thioUTP, and/or PSB1114.

67. The composition of claim 61, wherein the purinergic receptor-activating compound activates the P2Y1 purinergic receptor

68. The composition of claim 67, wherein the purinergic receptor-activating compound that activates the P2Y1 purinergic receptor comprises MRS2170, MRS2267, MRS2279, [3H]2MeSADP, MRS2365, 2-CI-ADP(α-BH3), compound 3a, ADPβS, Ap3a, Ap5a, 2′,3′-ddATP, dATPαS, ATPγS, 2MeSATP, ATP, ADP, and/or [35S]ADPβS.

69. The composition of claim 61, wherein the purinergic receptor-activating compound activates the P2Y14 purinergic receptor.

70. The composition of claim 69, wherein the purinergic receptor-activating compound that activates the P2Y14 purinergic receptor comprises a uridine diphosphate (UDP), a UDP-sugar, an α.β-methylene-2-thio-UDP, an MRS4183, an MRS2905, a 2-thio-UDP, an MRS2802, and/or an MRS2690.

71. The composition of claim 70, wherein the UDP-sugar comprises UDP-glucose, UDP-galactose, UDP-glucoronic acid, and/or UDP-N-acetylglucosamine.

72. The composition of claim 61, further comprising a therapeutically effective amount of an inhibitor comprising a NOD2 inhibitor, a Rho GTPase inhibitor, and/or an miR29c inhibitor wherein the therapeutically effective amount(s) promotes thymic function.

73. The composition of claim 72, wherein the therapeutically effective amount(s) that promotes thymic function promotes thymic regeneration.

74. The composition of claim 72, wherein the NOD2 inhibitor comprises ponatinib, regorafenib, gefitinib, curcumin, a sesquiterpene lactone, a pseudopterosin, a polyunsaturated fatty acid, a benzimidazole diamide, and/or a hydrophenalene-chromium complex.

75. The composition of claim 74, wherein the sesquiterpene lactone comprises parthenolide and/or helenalin.

76. The composition of claim 74, wherein the pseudopterosin comprises pseudopterosin A.

77. The composition of claim 74, wherein the polyunsaturated fatty acid comprises docosahexaenoic acid (DHA) and/or eicosapentaenoic acid (EPA).

78. The composition of claim 74, wherein the benzimidazole diamide comprises GSK669 and/or GSK717.

79. The composition of claim 72, wherein the Rho GTPase inhibitor comprises isoflavones, (E)-3-(3-(ethyl(quinolin-2-yl)amino)phenyl)acrylic acid, (E)-3-(3-(butyl(quinolin-2-yl)amino)phenyl)acrylic acid, C3 transferase, ZCL 278, Rhosin hydrochloride, ML 141, CASIN, p120 catenin, MLS000532223, and/or MLS000573151.

80. The composition of claim 72, wherein the Rho GTPase inhibitor comprises a RhoA inhibitor and/or a Rac1 inhibitor.

81. The composition of claim 80, wherein the Rac1 inhibitor comprises EHT 1864, Rac1 Inhibitor W56, NSC 23766, EHop 016, 6-mercaptopurine (6-MP), and/or 6-thioguanosine-5′-triphosphate (6-T-GTP).

82. The composition of claim 72, wherein the miR29c inhibitor comprises a complementary interfering RNA sequence.

83. The composition of claim 72, wherein the miR29c inhibitor comprises SEQ ID NO: 5.

84. The composition of claim 72, wherein the miR29c inhibitor comprises a PPAR-γ agonist.

85. The composition of claim 84, wherein the PPAR-γ agonist comprises pioglitazone; 15-deoxy-delta-12,14-PGJ₂; and/or thiazolidinedione.

86. The composition of claim 61, wherein the composition is labeled for use to treat a subject in need of promoted thymic function or at risk for needing promoted thymic function.

87. The composition of claim 61, wherein the composition is labeled for use to treat a subject in need of promoted thymic regeneration or at risk for needing promoted thymic regeneration.

88. The composition of claim 86, wherein the subject is in need of promoted thymic function or is at risk of needing promoted thymic function based on age or an immune-compromised status due to a treatment.

89. The composition of claim 87, wherein the subject is in need of promoted thymic regeneration or is at risk of needing promoted thymic regeneration based on age or an immune-compromised status due to a treatment.

90. The composition of claim 86, wherein the subject is in need of promoted thymic function or is at risk of needing promoted thymic function based on infection or a cancer treatment.

91. The composition of claim 87, wherein the subject is in need of promoted thymic regeneration or is at risk of needing promoted thymic regeneration based on infection or a cancer treatment.
